(19) United States
(12) Patent Application Publication
(10) Pub. No.: US 2015/0034924 A1
(43) Pub. Date: Feb. 5, 2015

(54) NOVEL TRIPHENYLENE DERIVATIVES AND ORGANIC ELECTROLUMINESCENT DEVICES USING SAID DERIVATIVES

(71) Applicant: HODOGAYA CHEMICAL CO., LTD., Tokyo (JP)

(72) Inventors: Norimasa Yokoyama, Ibaraki (JP); Naoaki Kabasawa, Tokyo (JP); Hiroshi Ookuma, Tokyo (JP); Shuichi Hayashi, Tokyo (JP); Eiji Takahashi, Kanagawa (JP)

(73) Assignee: HODOGAYA CHEMICAL CO., LTD., Tokyo (JP)

(21) Appl. No.: 14/349,438

(22) PCT Filed: Oct. 12, 2012
(86) PCT No.: PCT/JP2012/076434

§ 371 Date: Apr. 3, 2014

(30) Foreign Application Priority Data
Oct. 24, 2011 (JP) 2011-232794

(51) Int. Cl. H01L 51/00; H01L 51/50

(52) U.S. Cl. CPC H01L 51/006; H01L 51/0061; H01L 51/5056; H01L 51/5096; H01L 51/5088; H01L 51/5012; H01L 51/0054; H01L 51/0058; H01L 51/0068
USPC 257/40; 564/426; 564/427; 549/43

(57) ABSTRACT

Triphenylene derivatives represented by the following general formula (1),

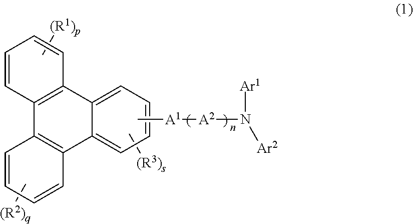

wherein Ar¹ and Ar² are aromatic groups.

The compounds have a structure in which an aromatic tertiary amine is introduced into a triphenylene ring. Owing to this structure, the compounds exhibit (A) favorable hole injection property, (B) large hole mobility, (C) excellent electron blocking power, (D) stability in their thin-film form, and (E) excellent heat resistance. The compounds are useful as hole-transporting materials for use in the organic EL devices.

### TECHNICAL FIELD

This invention relates to novel compounds (triphenylene derivatives) adapted to organic electroluminescent devices which are spontaneously luminous devices that can be favorably used for a variety of display devices, and to an organic electroluminescent device provided with organic layers that contain the above compounds.

### BACKGROUND ART

An organic electroluminescent device (hereinafter often called organic EL device) is a spontaneously luminous device which features higher brightness and higher legibility than those of the liquid crystal devices enabling vivid display to be attained and has, therefore, been vigorously studied.

In 1987, C. W. Tang et al. of the Eastman Kodak Co. have developed a device of a layer-laminated structure comprising various kinds of materials to bear individual roles, and have put an organic EL device using organic materials into a practical use. The above organic EL device is constituted by laminating layers of a fluorescent body capable of transporting electrons and an aromatic amine compound capable of transporting holes. Upon injecting both electric charges into the layer of the fluorescent body to emit light, the device is capable of attaining a brightness of as high as 1000 cd/m² or more with a voltage of not higher than 10 V.

So far, very many improvements have been made to put the organic electroluminescent device to practical use. For example, the organic EL device has been widely known having a structure comprising an anode, a hole injection layer, a hole-transporting layer, a luminous layer, an electron-transporting layer, an electron injection layer and a cathode which are arranged in this order on a substrate more finely dividing their roles than ever before. The device of this kind is achieving a high efficiency and a high durability.

To further improve the luminous efficiency, attempts have been made to utilize triplet excitons and study has been forwarded to utilize a luminous phosphor.

The luminous layer is, usually, produced by doping an electron-transporting compound called host material with a fluorescent body or a luminous phosphor. Properties such as efficiency and durability of the organic EL device are greatly affected by the selection of organic materials forming the layers of the device.

In the organic EL device, the electric charges injected from the two electrodes recombine together in the luminous layer to emit light. Here, what is important is that how the two electric charges of holes and electrons be efficiently handed over to the luminous layer. For instance, probability of recombination of holes and electrons can be improved by enhancing the hole injection property while enhancing the electoron blocking property blocking the electrons that are injected through the cathode and, besides, a high luminous efficiency can be realized by confining the formed excitons in the luminous layer. Therefore, the hole-transporting material plays an important role, and it has been desired to provide a hole-transporting material that has high hole injection property, large hole mobility, high electron-blocking power and large durability against the electrons.

Heat resistance and amorphous property of the materials are also important concerning the life of the device. The material having low heat resistance undergoes the hydrolysis even at a low temperature due to the heat generated when the device is driven, and is degraded. The material having low amorphous property in the form of a thin film undergoes crystallization in short periods of time causing the device to be degraded. Therefore, the material that is used must have properties such as high heat resistance and good amorphous property.

As the hole-transporting materials used for the organic EL devices, there have been known an N,N′-diphenyl-N,N′-di(α-naphthyl)benzidine (hereinafter abbreviated as NPD) and a variety of aromatic amine derivatives (e.g., see a patent document 1 and a patent document 2).

The NPD has a good hole-transporting power but has a glass transition point (Tg) that serves as an index of heat resistance of as low as 96° C. and causes a decrease in the properties of the device due to crystallization under high-temperature conditions. Some of the aromatic amine derivatives described in the patent documents 1 and 2 have hole mobilities which are as excellent as 10⁻³ cm²/Vs or higher but insufficient electron-blocking power permitting part of the electrons to pass through the luminous layer and failing to meet the expectations of higher luminous efficiency. Therefore, it has been desired to provide materials having higher electron-blocking power and higher heat resistance maintaining stability in their thin-film form to attain further improved efficiencies.

As compounds improving properties such as heat resistance and hole injection property, patent documents 3 and 4 are proposing arylamine compounds A and B having substituted triphenylene structures as represented by the following formulas,

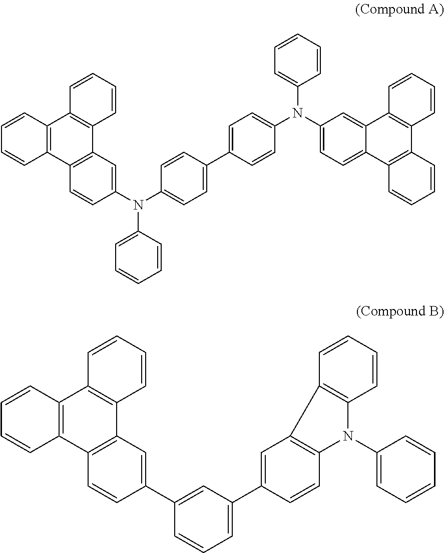 [SMILES: c1ccc(N(c2ccc(-c3ccc(N(c4ccccc4)c4ccc5c6ccccc6c6ccccc6c5c4)cc3)cc2)c2ccc3c4ccccc4c4ccccc4c3c2)cc1 ; c1ccc(-n2c3ccccc3c3cc(-c4cccc(-c5ccc6c7ccccc7c7ccccc7c6c5)c4)ccc32)cc1]

The devices using these compounds for forming the hole injection layer or the hole-transporting layer have improved heat resistance and luminous efficiency which, however, are not still satisfactory. Besides, their driving voltages have not been sufficiently lowered and their current efficiencies are not satisfactory, either, leaving problem in their amorphous property, too. Therefore, it has been urged to provide compounds that make it possible to further lower the driving voltage and to further improve the luminous efficiency yet maintaining higher amorphous property.

### PRIOR ART DOCUMENTS

### Patent Documents

Patent document 1: JP-A-8-48656
Patent document 2: Japanese Patent No. 3194657
Patent document 3: WO2010/002850
Patent document 4: WO2011/081423

### OUTLINE OF THE INVENTION

### Problems that the Invention is to Solve

To achieve the above objects, the present inventors have paid attention to the facts that an aromatic tertiary amine structure has a high hole injection/transporting power, that a triphenylene ring structure imparts good heat resistance and thin-film stability, have designed various compounds having the triphenylene ring structure, have chemically synthesized them, attempted to fabricate various organic electroluminescent devices using these compounds, keenly evaluated the properties of the devices, have confirmed that high efficiencies and excellent durability can be realized, and have thus completed the present invention.

According to the present invention, there are provided triphenylene derivatives represented by the following general formula (1),

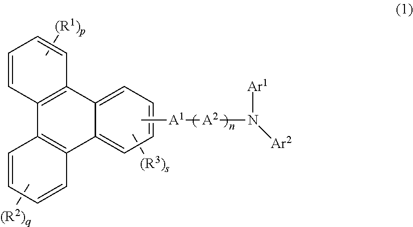

wherein,
p and q are, respectively, integers of 0 or 1 to 4,
s is an integer of 0 or 1 to 3,
n is an integer of 0, 1 or 2,
Ar¹ and Ar² are, respectively, aromatic hydrocarbon groups or aromatic heterocyclic groups and, wherein, Ar¹ and Ar² may form a ring by being bonded together via a single bond, via a methylene group that may have a substituent, via an oxygen atom or via a sulfur atom,
R¹, R² and R³ are, respectively, deuterium atoms, fluorine atoms, chlorine atoms, cyano groups, nitro groups, alkyl groups having 1 to 6 carbon atoms, cycloalkyl groups having 5 to 10 carbon atoms, alkyloxy groups having 1 to 6 carbon atoms, cycloalkyloxy groups having 5 to 10 carbon atoms, aromatic hydrocarbon groups, aromatic heterocyclic groups or aryloxy groups,
A¹ and A² are, respectively, divalent aromatic hydrocarbon groups or divalent aromatic heterocyclic groups,
if n is 0, then A¹ and Ar¹ may form a ring by being bonded together via a single bond, via a methylene group that may have a substituent, via an oxygen atom or via a sulfur atom,
if n is 1, then A¹ or A² and Ar¹ may form a ring by being bonded together via a single bond, via a methylene group that may have a substituent, via an oxygen atom or via a sulfur atom, and
if n is 2, then the plurality of A² may be different from each other, and A¹ or A² and Ar¹ may form a ring by being bonded together via a single bond, via a methylene group that may have a substituent, via an oxygen atom or via a sulfur atom.

According to the present invention, further, there is provided an organic electroluminescent device having a pair of electrodes and at least one organic layer interposed therebetween, wherein at least one of the organic layers contains the triphenylene derivative.

In the organic EL device of the invention, the organic layer containing the triphenylene derivative is, for example, a hole-transporting layer, an electron-blocking layer, a hole injection layer or a luminous layer.

The triphenylene derivative of the present invention represented by the above general formula (1) is a novel compound, has a structure in which an aromatic tertiary amine is introduced into a triphenylene ring, and features the following properties owing to the above structure.
(A) Favorable hole injection property.
(B) Large hole mobility.
(C) Excellent electron-blocking power.
(D) Stability in its thin-film form (excellent amorphous property).
(E) Excellent heat resistance.

Therefore, the triphenylene derivative of the present invention is useful as a hole-transporting material for use in the organic EL devices, remains stable in its thin-film form, and can be used specifically as an organic layer for use in the organic EL devices to impart the following properties to the organic EL devices.
(F) A high luminous efficiency and a high power efficiency.
(G) A low luminescence start voltage.
(H) A low practical driving voltage.
(I) A long service life of the device (large durability).

For instance, the organic EL device forming the hole injection layer and/or the hole-transporting layer by using the triphenylene derivative of the invention, features high hole injection/mobility, high electron-blocking power and high stability against the electrons, making it possible to confine the formed excitons in the luminous layer and to improve the probability of recombination of the holes with the electrons to attain a high luminous efficiency. Moreover, the driving voltage is low contributing to improving the durability.

Further, the organic EL device having an electron-blocking layer formed by using the triphenylene derivative of the invention features excellent electron-blocking power and hole-transporting capability and, therefore, requires a decreased driving voltage yet maintaining a high luminous efficiency and, besides, features an improved resistance against the electric current and an improved maximum brightness.

Further, the triphenylene derivative of the invention features excellent hole-transporting capability and a wide band gap as compared to the conventional materials and can, therefore, be used as a host material for the luminous layer. By using the triphenylene derivative of the invention as a luminous layer which, further, carries a fluorescent luminous body or a phosphorus luminous body called dopant thereon, it is made possible to lower the driving voltage of the organic EL device and to improve the luminous efficiency.

As described above, the triphenylene derivative of the present invention is very useful as a material for constituting the hole injection layer, hole-transporting layer, electron-blocking layer or luminous layer of the organic EL device, and works to improve the luminous efficiency and the power efficiency of the organic EL device, to lower the practical driving voltage and to increase the durability.

### BRIEF DESCRIPTION OF THE DRAWINGS

FIG. 1 is a ¹H-NMR chart of a compound (compound 66) of Example 1

FIG. 2 is a ¹H-NMR chart of a compound (compound 15) of Example 2

FIG. 3 is a ¹H-NMR chart of a compound (compound 67) of Example 3.

FIG. 4 is a ¹H-NMR chart of a compound (compound 79) of Example 4.

FIG. 5 is a ¹H-NMR chart of a compound (compound 80) of Example 5.

FIG. 6 is a ¹H-NMR chart of a compound (compound 81) of Example 6

FIG. 7 is a ¹H-NMR chart of a compound (compound 82) of Example 7.

FIG. 8 is a ¹H-NMR chart of a compound (compound 83) of Example 8

FIG. 9 is a ¹H-NMR chart of a compound (compound 84) of Example 9.

FIG. 10 is a ¹H-NMR chart of a compound (compound 85) of Example 10.

FIG. 11 is a ¹H-NMR chart of a compound (compound 86) of Example 11.

FIG. 12 is a ¹H-NMR chart of a compound (compound 87) of Example 12.

FIG. 13 is a ¹H-NMR chart of a compound (compound 46) of Example 13.

FIG. 14 is a ¹H-NMR chart of a compound (compound 88) of Example 14.

FIG. 15 is a ¹H-NMR chart of a compound (compound 89) of Example 15.

FIG. 16 is a ¹H-NMR chart of a compound (compound 90) of Example 16.

FIG. 17 is a view illustrating the structure of layers of an organic EL device.

### MODES FOR CARRYING OUT THE INVENTION

The triphenylene derivative of the present invention is represented by the following formula (1) and has a structure in which an aromatic tertiary amine is bonded to a triphenylene ring via a divalent group,

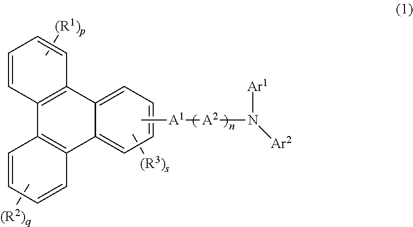

In the above general formula (1), p and q represent the numbers of the substituents R¹ and R² bonded to the triphenylene ring and are, respectively, integers of 0 or 1 to 4.

Further, s represents the number of the substituent R³ bonded to the triphenylene ring and is an integer of 0 or 1 to 3.

Further, n represents the number of the divalent group A² that is present between a nitrogen atom of the aromatic amine and the triphenylene ring, and is an integer of 0, 1 or 2.

Moreover, Ar¹ and Ar² that are bonded to the nitrogen atom in the general formula (1) are, respectively, aromatic hydrocarbon groups or aromatic heterocyclic groups. The aromatic hydrocarbon groups and aromatic heterocyclic groups may have a single-ring structure or a condensed multi-ring structure.

Examples of the aromatic group include phenyl group, biphenylyl group, terphenylyl group, naphthyl group, anthryl group, phenanthryl group, fluorenyl group, indenyl group, pyrenyl group, perylenyl group, fluoranthenyl group, triphenylenyl group, pyridyl group, furanyl group, pyranyl group, thienyl group, quinolyl group, isoquinolyl group, benzofuranyl group, benzothienyl group, indolyl group, carbazolyl group, benzoxazolyl group, benzothiazolyl group, quinoxalyl group, benzoimidazolyl group, pyrazolyl group, dibenzofuranyl group, dibenzothienyl group and carbolynyl group.

Of the above aromatic groups (aromatic hydrocarbon groups and aromatic heterocyclic groups), preferred aromatic heterocyclic groups are oxygen-containing aromatic heterocyclic groups such as furanyl group, benzofuranyl group, benzoxazolyl group and dibenzofuranyl group; and sulfur-containing aromatic heterocyclic groups such as thienyl group, benzothienyl group, benzothiazolyl group and dibenzothienyl group. Among them, the sulfur-containing aromatic heterocyclic group is specifically preferred, and the dibenzothienyl group is particularly preferred.

As the aromatic hydrocarbon group, there can be preferably used phenyl group, biphenylyl group, naphthyl group and fluorenyl group.

Further, the above aromatic group may have a substituent. As the substituent, there can be exemplified deuterium atom; cyano group; nitro group; halogen atom such as fluorine atom, chlorine atom, bromine atom or iodine atom; straight-chain or branched alkyl groups having 1 to 6 carbon atoms, such as methyl group, ethyl group, n-propyl group, isopropyl group, n-butyl group, isobutyl group, tert-butyl group, n-pentyl group, isopentyl group, neopentyl group and n-hexyl group; straight-chain or branched alkyloxy groups having 1 to 6 carbon atoms, such as methyloxy group, ethyloxy group and propyloxy group; alkenyl groups such as ally group; aralkyl groups such as benzyl group, naphthylmethyl group and phenetyl group; aryloxy groups such as phenyloxy group and tolyloxy group; arylalkyloxy groups such as benzyloxy group and phenetyloxy group; aromatic hydrocarbon groups such as phenyl group, biphenylyl group, terphenylyl group, naphthyl group, anthracenyl group, phenanthryl group, fluorenyl group, indenyl group, pyrenyl group, perylenyl group, fluoranthenyl group and triphenylenyl group; aromatic heterocyclic groups such as pyridyl group, furanyl group, pyranyl group, thienyl group, pyrolyl group, quinolyl group, isoquinolyl group, benzofuranyl group, benzothienyl group, indolyl group, carbazolyl group, benzoxazolyl group, benzothiazolyl group, quinoxalyl group, benzoimidazolyl group, pyrazolyl group, dibenzofuranyl group, dibenzothienyl group and carbolinyl group; arylvinyl groups such as stylyl group and naphthylvinyl group; and acyl groups such as acetyl group and benzoyl group. These substituents may, further, have a substituent like trifluoromethyl group, or the substituents bonded to Ar¹ may be bonded together to form a ring, or the substituents bonded to Ar² may be bonded together to form a ring.

In the invention, the substituent possessed by the aromatic group is, preferably, a straight-chain or branched alkyl group having 1 to 6 carbon atoms and is, specifically, a methyl group or a tert-butyl group.

Further, the above Ar¹ and Ar² may form a ring by being bonded together via a single bond, via a methylene group that may have a substituent, via an oxygen atom or via a sulfur atom.

In the above general formula (1), R¹, R² and R³ that are bonded to the triphenylene ring are, respectively, deuterium atoms, fluorine atoms, chlorine atoms, cyano groups, nitro groups, alkyl groups having 1 to 6 carbon atoms, cycloalkyl groups having 5 to 10 carbon atoms, alkyloxy groups having 1 to 6 carbon atoms, cycloalkyloxy groups having 5 to 10 carbon atoms, aromatic hydrocarbon groups, aromatic heterocyclic groups or aryloxy groups.

Of the above R¹ to R³, the alkyl group having 1 to 6 carbon atoms may be a straight chain or branched. Its concrete examples include methyl group, ethyl group, n-propyl group, isopropyl group, n-butyl group, isobutyl group, tert-butyl group, n-pentyl group, isopentyl group, neopentyl group and n-hexyl group.

These alkyl groups may, further, have a substituent such as deuterium atom; fluorine atom, chlorine atom, cyano group, aryl group (e.g., phenyl group, naphthyl group, anthryl group, fluorenyl group, stylyl group, etc.) or aromatic heterocyclic group (pyridyl group, pyridoindolyl group, quinolyl group, benzothiazolyl group, etc.). For example, the above alkyl group may be such a group as trifluoromethyl group.

Further, the cycloalkyl groups having 5 to 10 carbon atoms, alkyloxy groups having 1 to 6 carbon atoms and cycloalkyloxy groups having 5 to 10 carbon atoms represented by R¹ to R³ may all be straight-chains or branched, and their concrete examples are as follows:

Cycloalkyl groups:
Cyclopentyl group, cyclohexyl group, 1-adamantyl group, 2-adamantyl group, etc.
   Alkyloxy groups:
Methyloxy group, ethyloxy group, n-propyloxy group, isopropyloxy group, n-butyloxy group, tert-butyloxy group, n-pentyloxy group, n-hexyloxy group, etc.
   Cycloalkyloxy groups:
Cyclopentyloxy group, cyclohexyloxy group, cycloheptyloxy group, cyclooctyloxy group, 1-adamantyloxy group, 2-adamantyloxy group, etc.

These cycloalkyl groups, alkyloxy groups and cycloalkyloxy groups, too, may have a substituent. As the substituent, there can be exemplified those which are the same as the substituents that are possessed by the above-mentioned aromatic hydrocarbon groups and aromatic heterocyclic groups Ar¹ and Ar².

The aromatic hydrocarbon groups and aromatic heterocyclic groups represented by R¹ to R³, too, are the same groups as those exemplified for Ar¹ and Ar², and may have a substituent, too.

As the aryloxy groups represented by R¹ to R³, there can be exemplified phenyloxy group, tolyloxy group, biphenyloxy group, terphenylyloxy group, naphthyloxy group, anthryloxy group, phenanthryloxy group, fluorenyloxy group, indenyloxy group, pyrenyloxy group and perylenyloxy group.

These aryloxy groups, too, may have a substituent, as a matter of course. As the substituent, there can be exemplified those which are the same as the substituents that may be possessed by the aromatic hydrocarbon groups and aromatic heterocyclic groups represented by Ar¹ and Ar².

In the general formula (1), A¹ and A² represent, respectively, divalent aromatic hydrocarbon groups or divalent aromatic heterocyclic groups via which the nitrogen atom of the aromatic amino group is bonded to the triphenylene ring. These divalent aromatic hydrocarbon groups and aromatic heterocyclic groups are not limited to those of the single-ring structure but may have a multi-ring structure to which a hydrocarbon ring or a heterocyclic ring is, further, bonded.

As the above divalent aromatic hydrocarbon groups, there can be exemplified those having an aromatic ring structure, such as benzene, biphenyl, terphenyl, tetrakisphenyl, styrene, naphthalene, anthracene, acenaphthalene, fluorene, phenanthrene, indane and pyrene and, particularly preferably, the divalent groups of the aromatic ring structure having benzene, biphenyl or fluorene.

As the divalent aromatic heterocyclic group, there can be exemplified those having a heterocyclic ring, such as pyridine, pyrimidine, triazine, furan, pyran, thiophene, quinoline, isoquinoline, benzofuran, benzothiophene, indoline, carbazole, benzooxazole, benzothiazole, quinoxaline, benzoimidazole, pyrazole, dibenzofuran, dibenzothiophene, naphthylidine, phenanthroline and acrydinine and, specifically, those having an oxygen-containing aromatic heterocyclic ring, such as furan, benzofuran, benzooxazole and dibenzofuran, as well as those having a sulfur-containing aromatic heterocyclic ring, such as thiophene, benzothiophene, benzothiazole and dibenzothiophene. Among them, those having the sulfur-containing aromatic heterocyclic ring are particularly preferred.

Either the above divalent aromatic hydrocarbon group or the divalent aromatic heterocyclic group may have a substituent which can be the same substituents as those that may be possessed by the aromatic hydrocarbon group and the aromatic heterocyclic group represented by Ar¹ and Ar².

In the above general formula (1), if n that represents the number of A² is 0 (i.e., if there is no A²), then A¹ and Ar¹ may form a ring by being bonded together via a single bond, via a methylene group that may have a substituent, via an oxygen atom or via a sulfur atom.

Further, if n is 1, then A¹ or A² and Ar¹ may form a ring by being bonded together via a single bond, via a methylene group that may have a substituent, via an oxygen atom or via a sulfur atom.

Further, if n is 2, then the plurality of A² may be different from each other, and A¹ or A² and Ar¹ may form a ring by being bonded together via a single bond, via a methylene group that may have a substituent, via an oxygen atom or via a sulfur atom.

The above triphenylene derivative is a novel compound and is synthesized, for example, in a manner as described below.

First, use is made of a triphenylene that corresponds to the triphenylene ring possessed by the triphenylene derivative of the general formula (1), and a portion (e.g., second position) to where the group A¹ of the triphenylene ring bonds is brominated to transform the bromine into a boronic acid or a boronic acid ester (e.g., see WO2010/002850).

The thus obtained boronic acid ester and brominated product of the amine corresponding to the aromatic amine portion possessed by the triphenylene derivative of the general formula (1), are subjected to the cross-coupling reaction such as the Suzuki coupling (e.g., see Chem. Rev., 95, 2457 (1995)) to synthesize the desired triphenylene derivative.

The obtained compound is refined by a column chromatography, by an adsorption refining using silica gel, activated carbon or activated clay, by a recrystallization method using a solvent or by a crystallization method. The compound is identified by the NMR analysis.

The above-mentioned triphenylene derivative of the present invention is, desirably, the one of which the n is zero in the general formula (1) and, specifically, of which the divalent group A¹ in the general formula (1) is a phenylene group that may have a substituent (specifically, that is not substituted). The above desirable triphenylene derivative is concretely represented by the following general formula (1a),

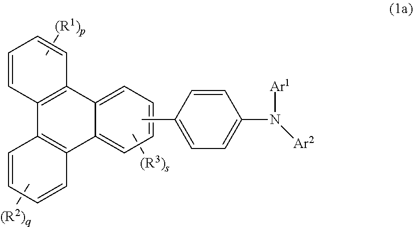

wherein,
p, q, s, Ar¹, Ar² and R¹ to R³ are as defined in the above general formula (1).

In the invention, further, also preferred is a triphenyllene derivative of which the divalent group A¹ is bonded to the second position of the triphenylene ring in the above general formula (1), or which is concretely represented by the following general formula (1′),

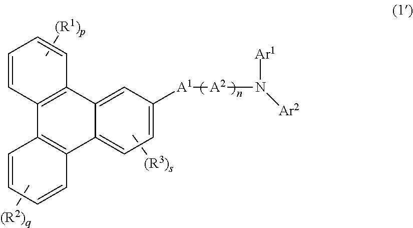

wherein,
p, q, s, n, Ar¹, Ar², R¹ to R³, A¹ and A² are as defined in the above general formula (1).

In the compound of the type in which the divalent group A¹ is bonded to the second position of the triphenylene ring, too, it is desired that n is zero and, specifically, that the divalent group A¹ is a phenylene group that may have a substituent (specifically, that is not substituted). The preferred compound of this kind is represented by, for example, the following general formula (1b),

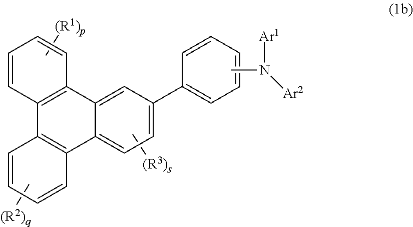

wherein,
p, q, s, Ar¹, Ar² and R¹ to R³ are as defined in the above general formula (1).

Further, among the triphenylene derivatives represented by the above general formula (1b), preferred is a compound in which the aromatic amino group (—NAr¹Ar²) is bonded to the p-position of the phenylene group (corresponds to A¹) that is bonded to the second position of the triphenylene ring or, concretely, a compound represented by the following general formula (1b-1).

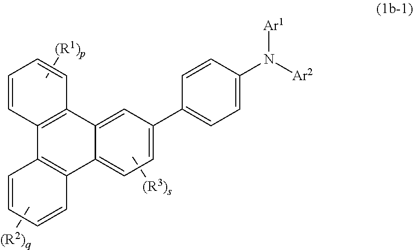

wherein,
p, q, s, Ar¹, Ar² and R¹ to R³ are as defined in the above general formula (1).

Described below are concrete examples among the triphenylene derivatives represented by the above general formula (1). In the following compounds, the compounds Nos. 1 and 2 are not listed.

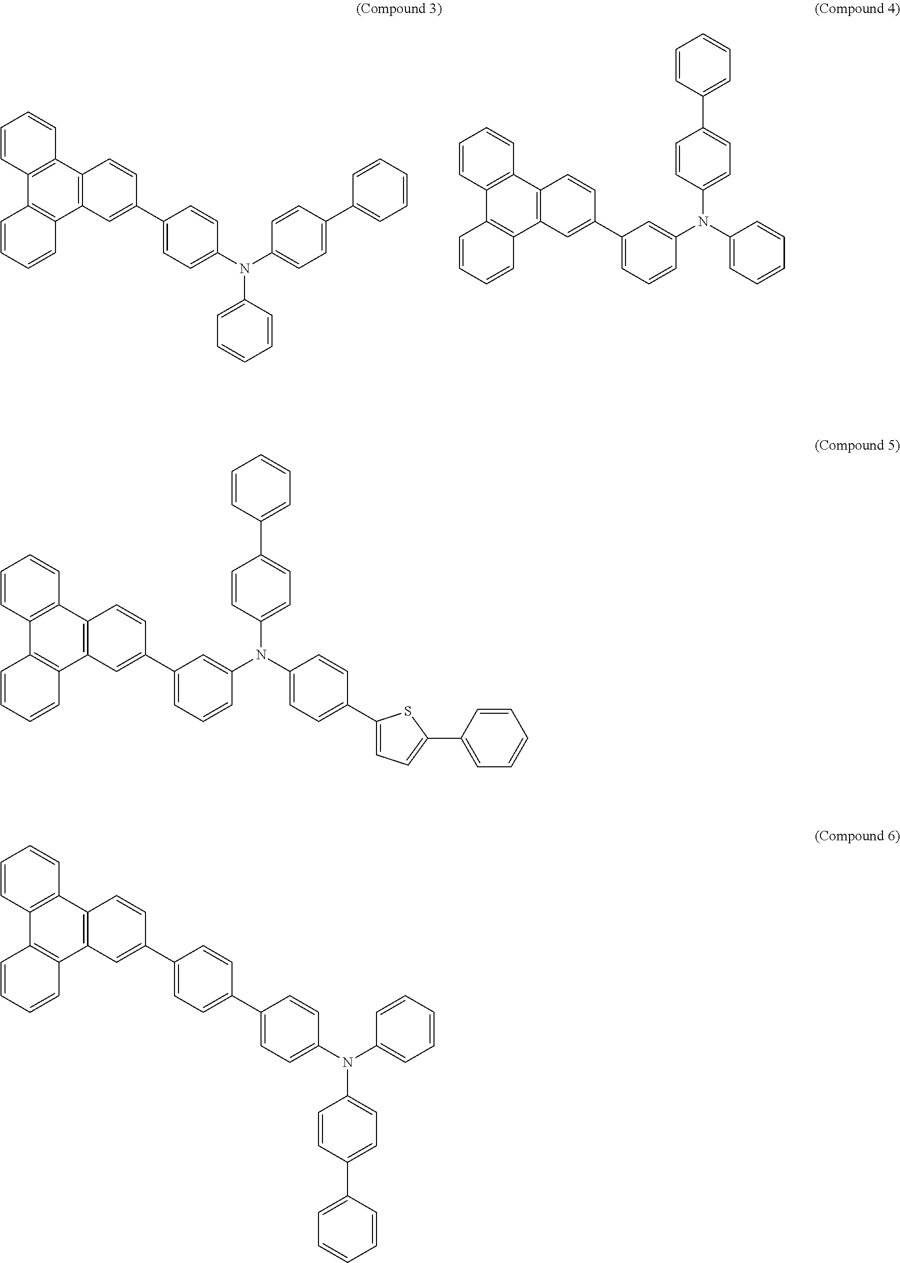

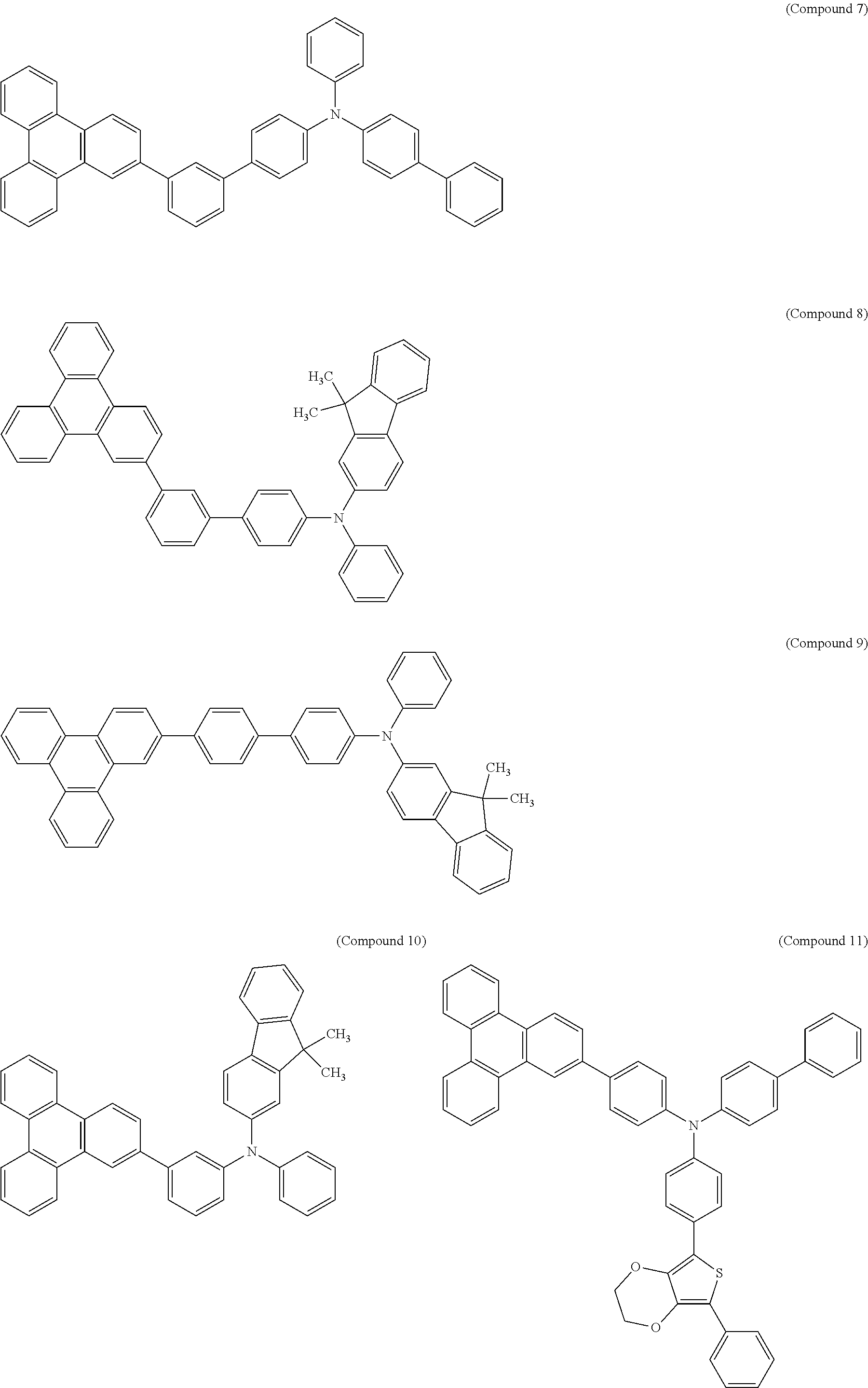

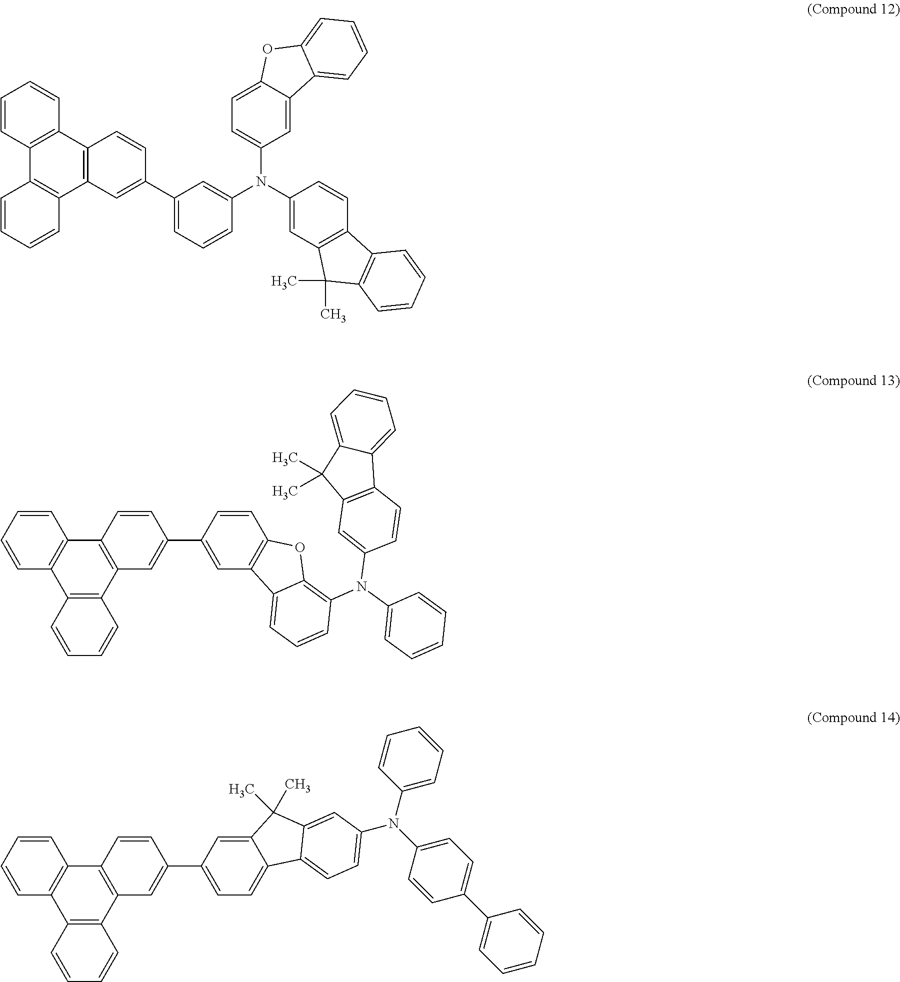

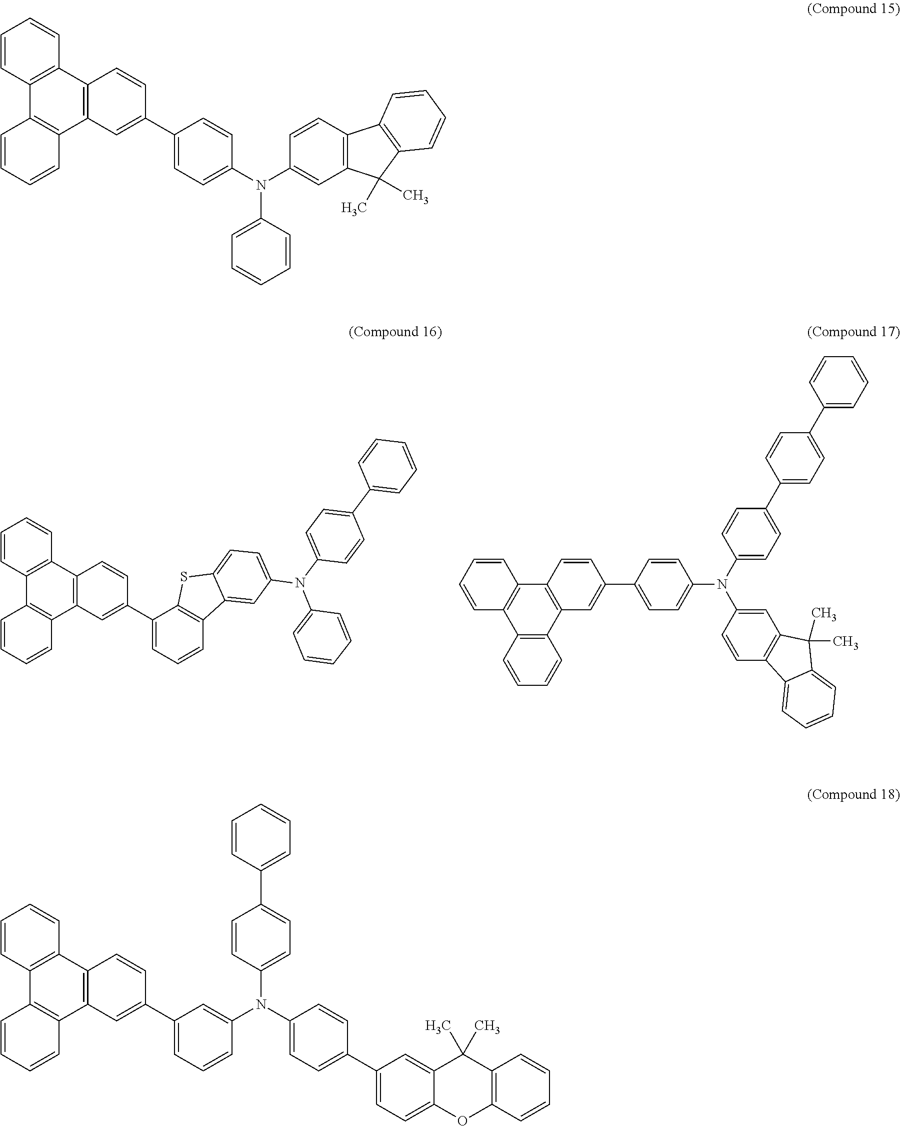

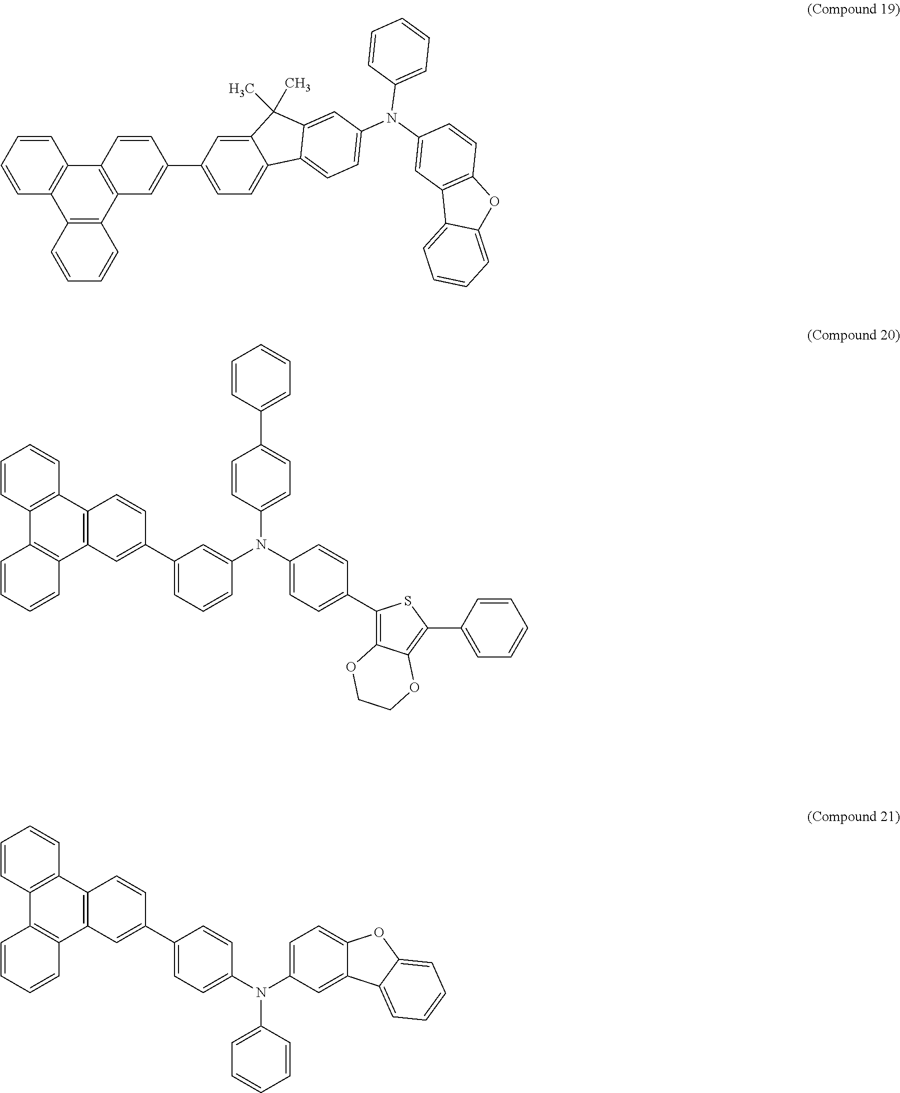

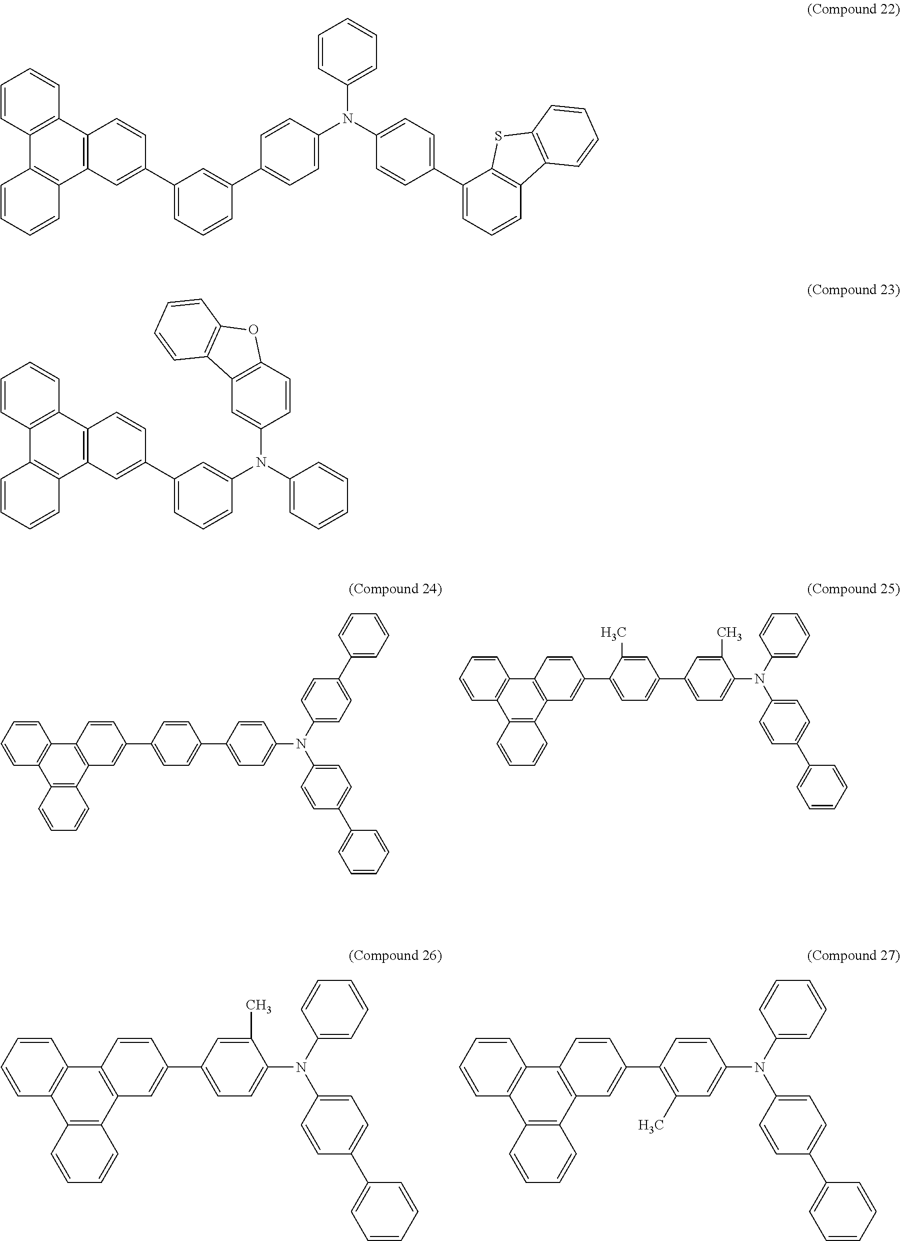

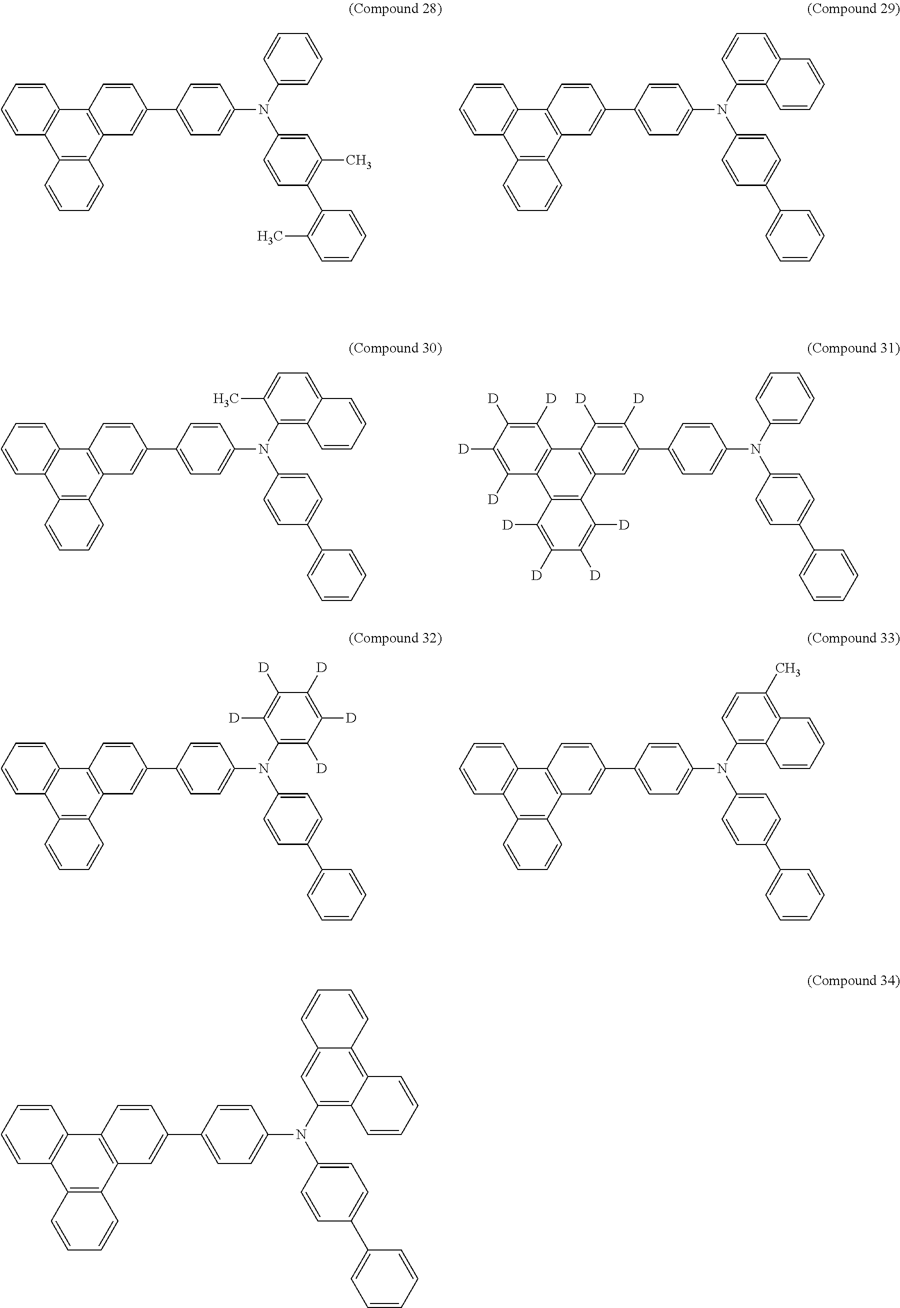

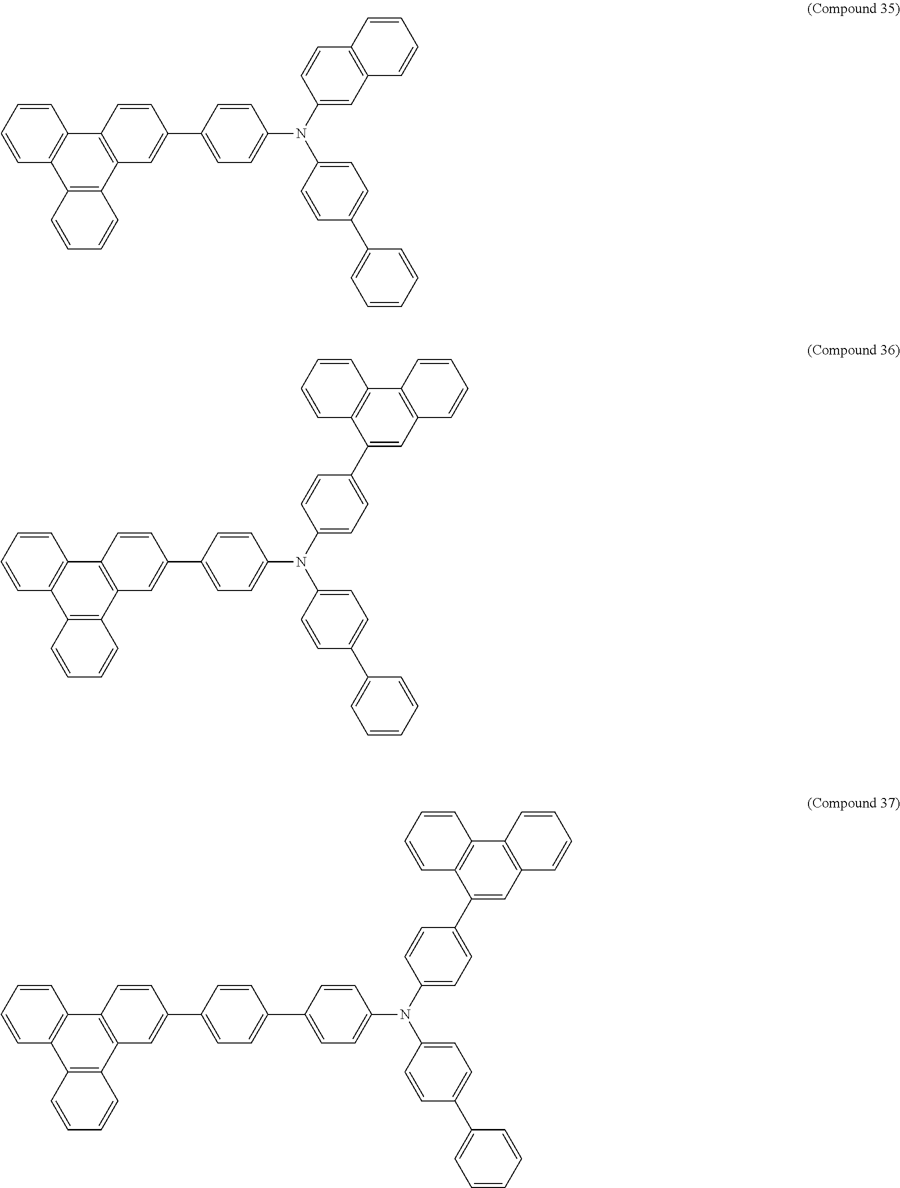

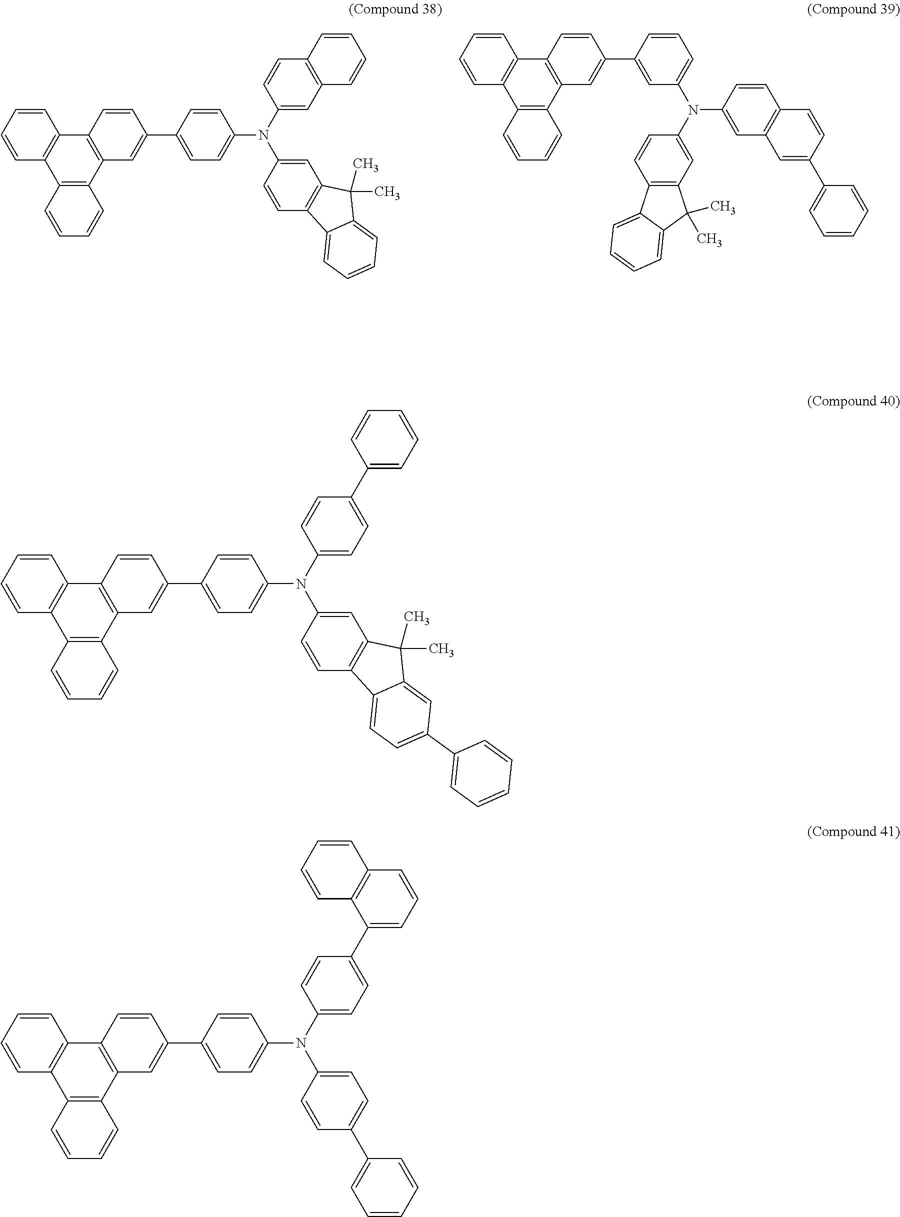

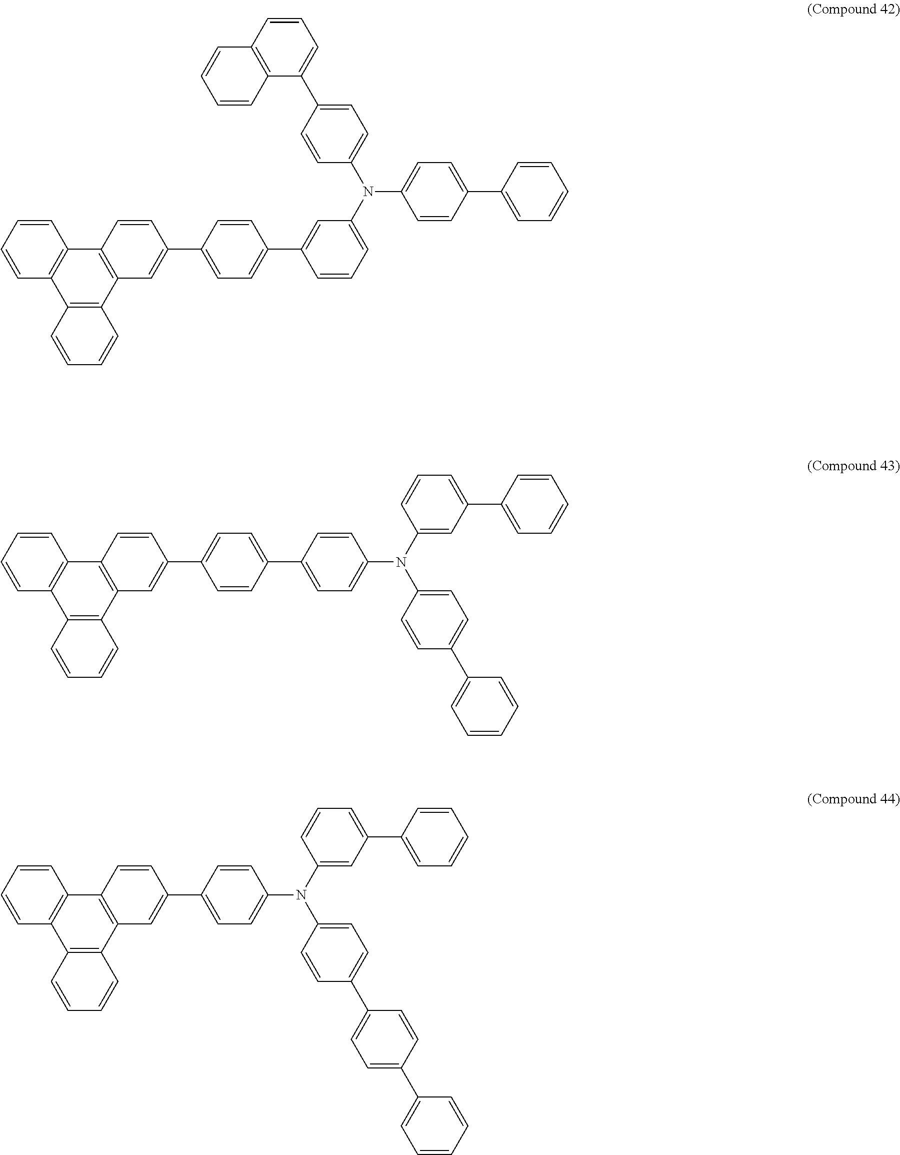

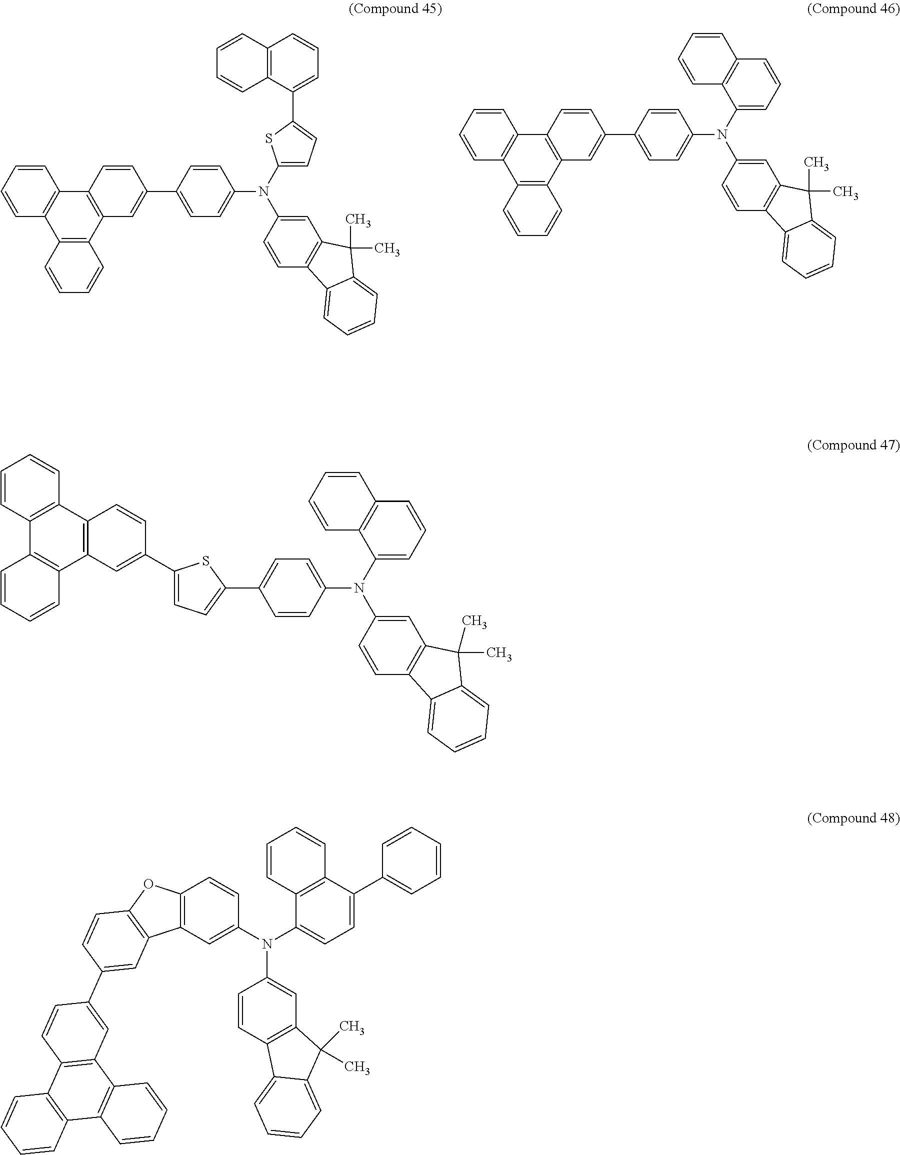

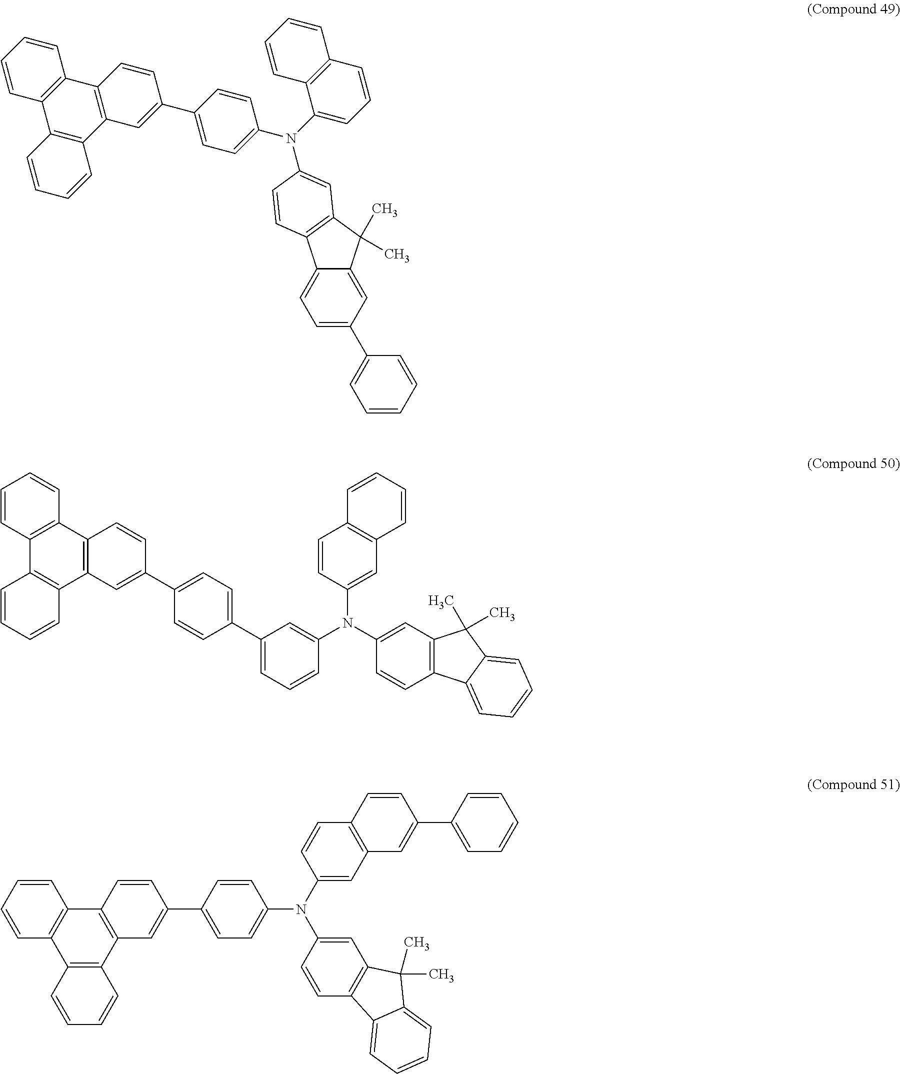

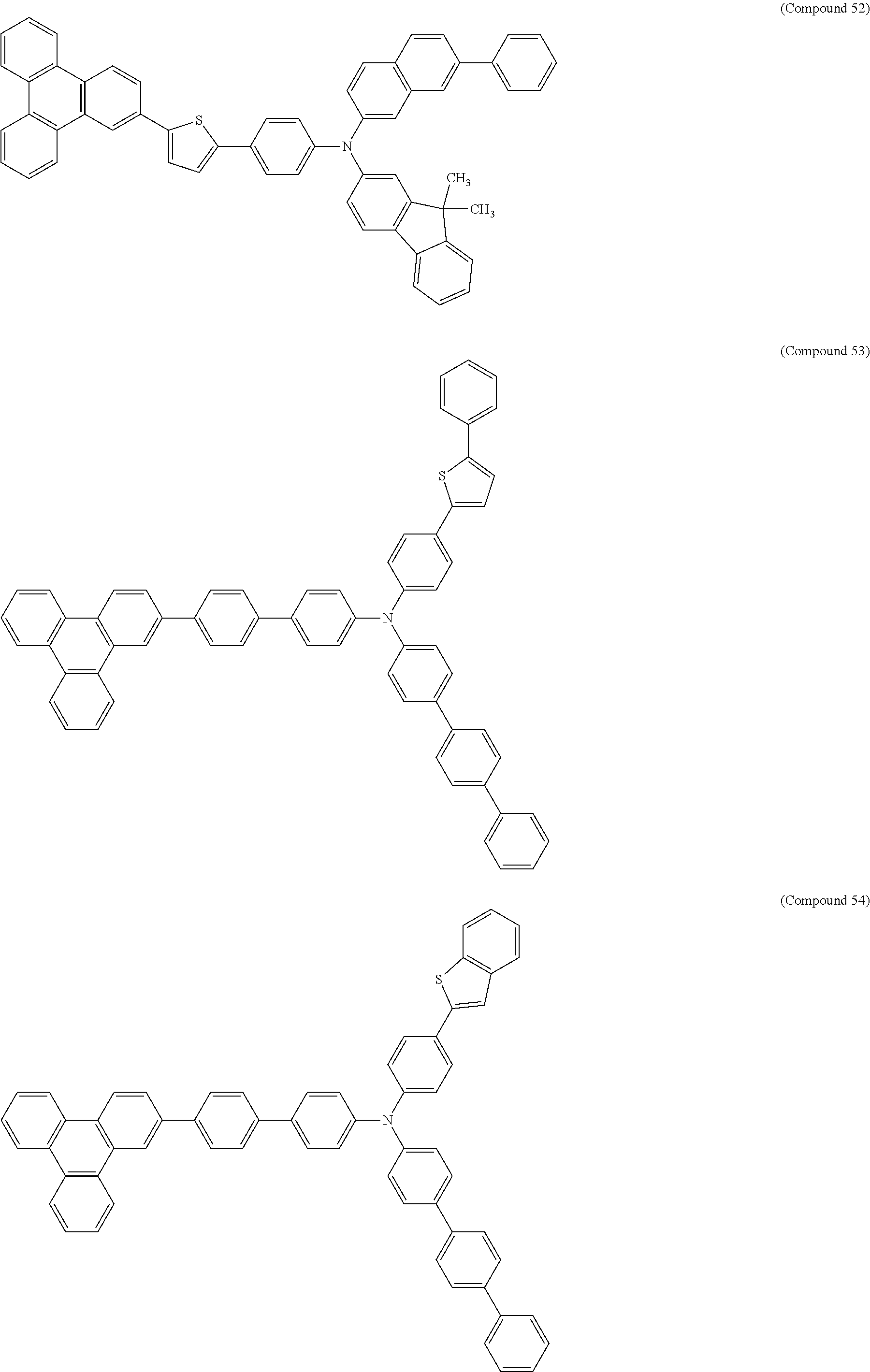

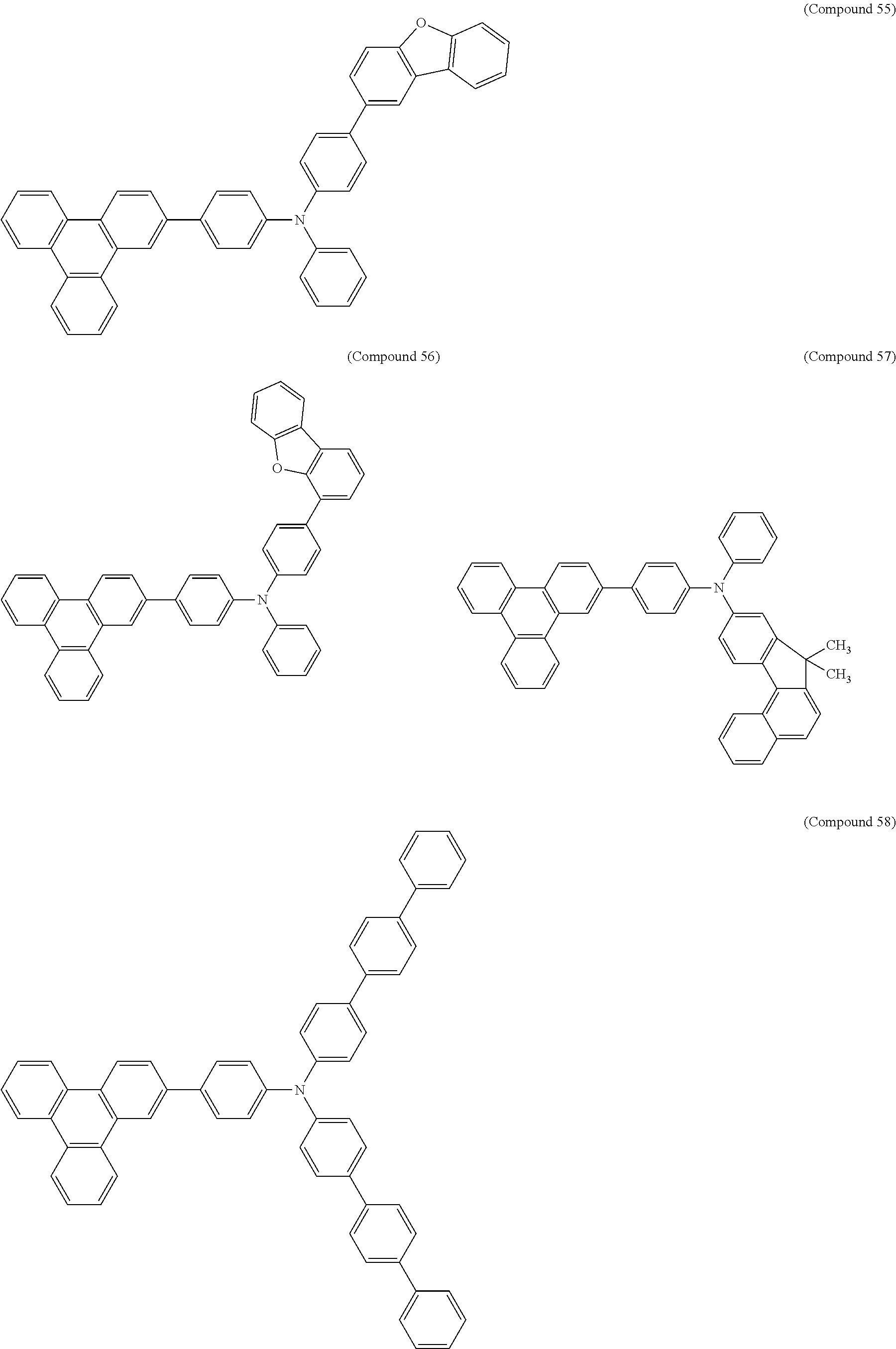

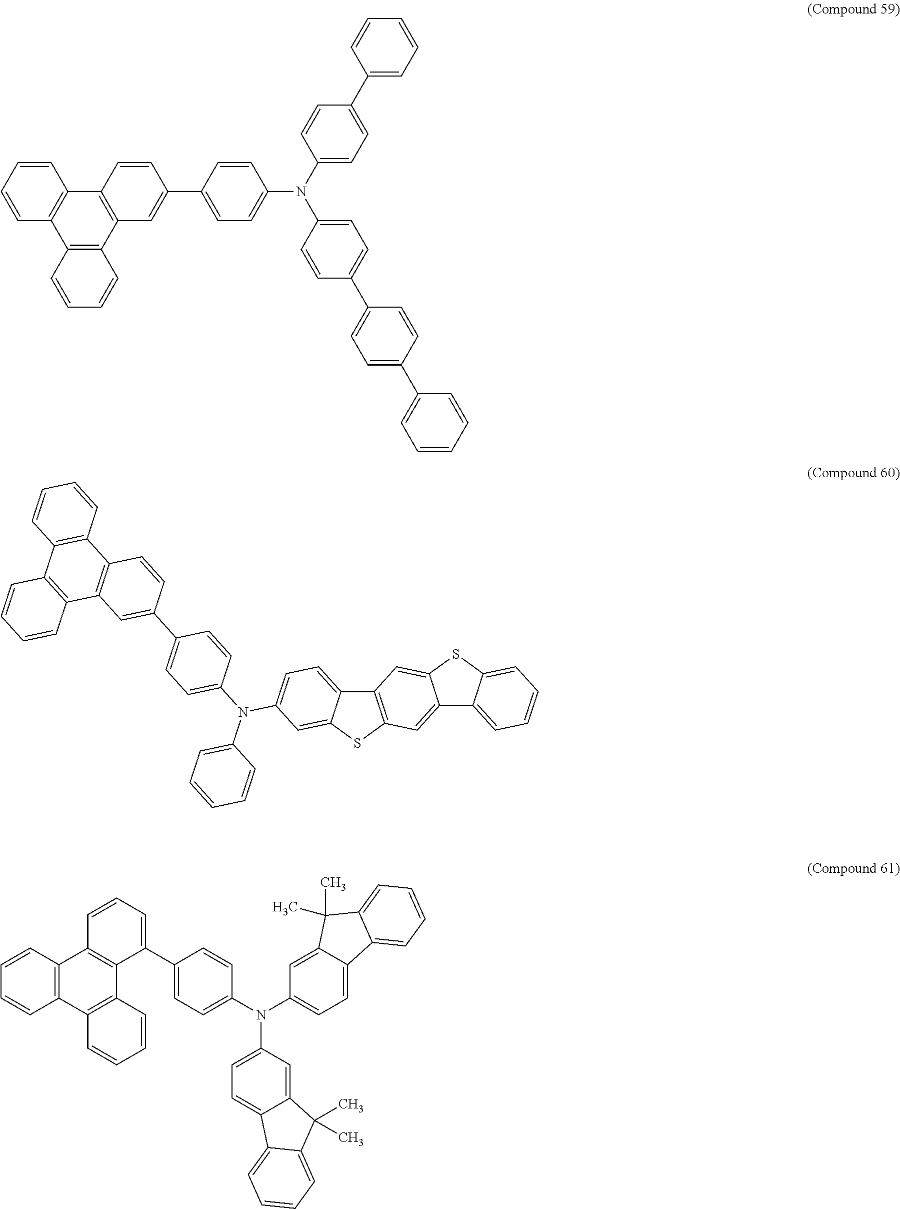

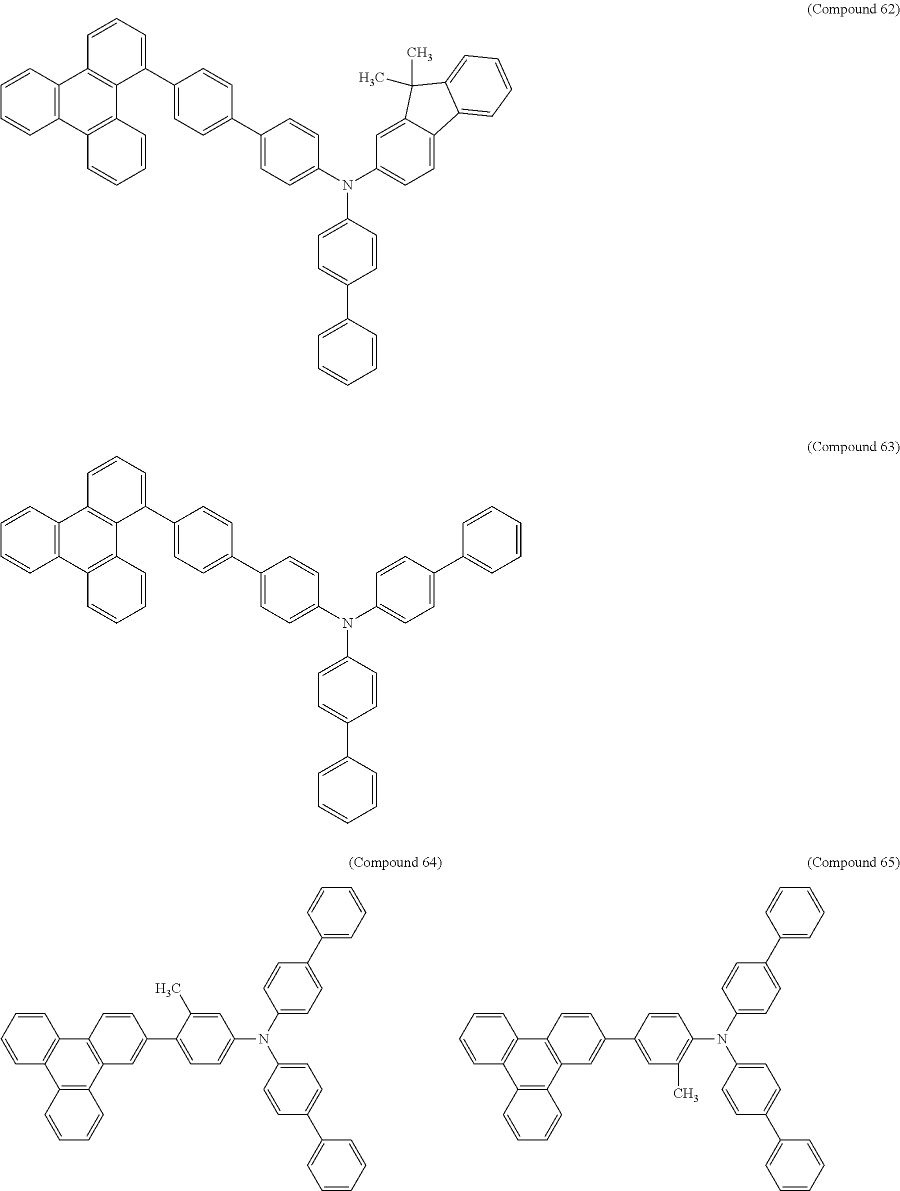

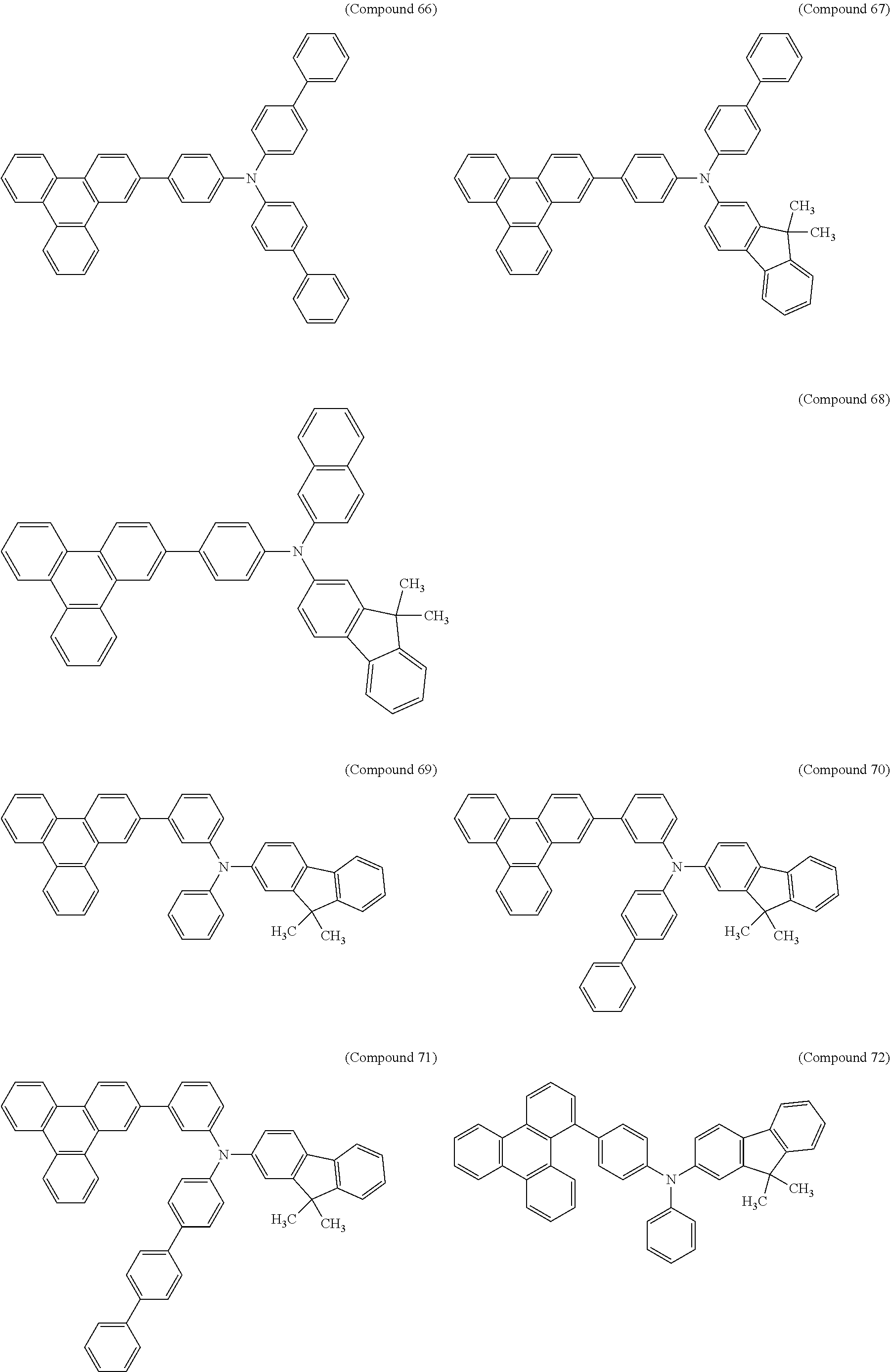

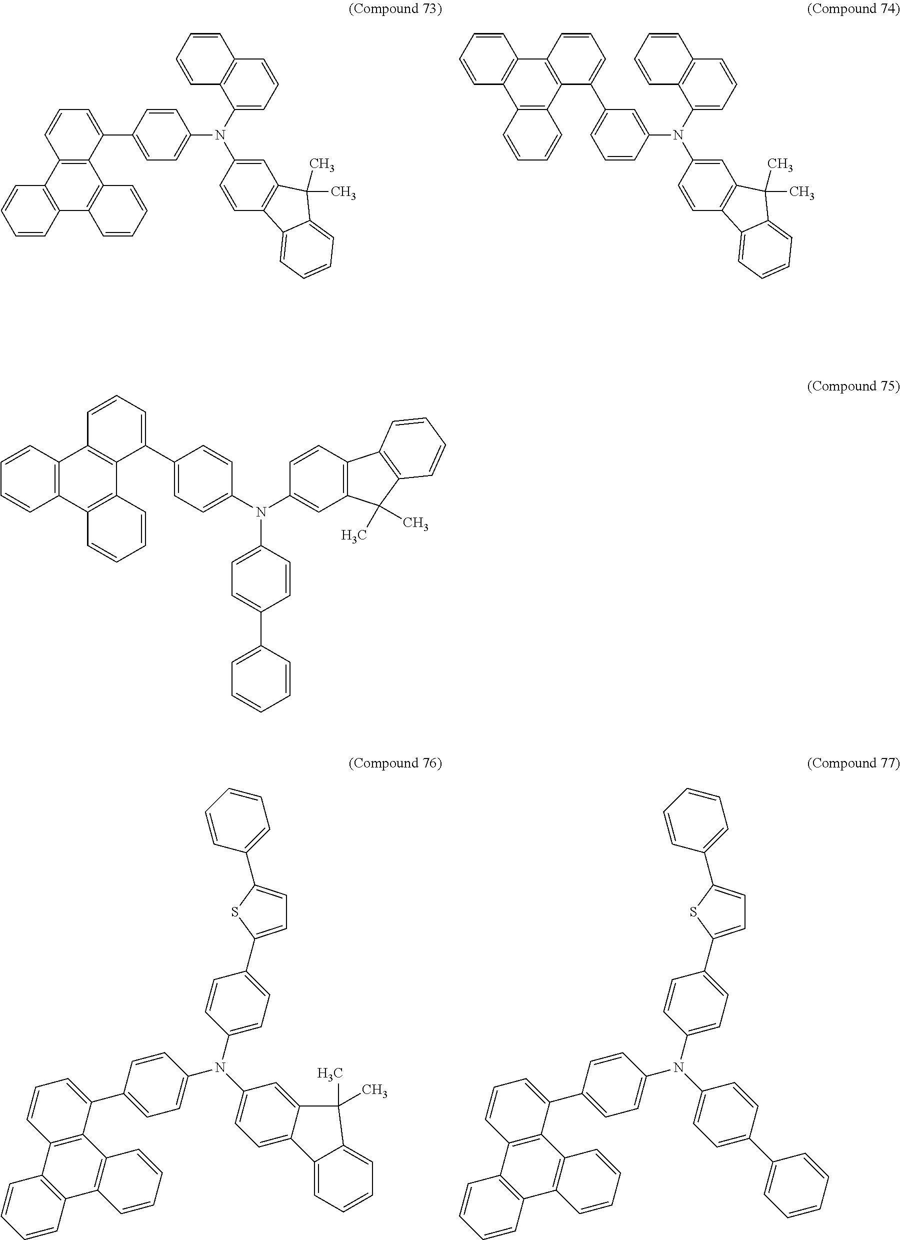

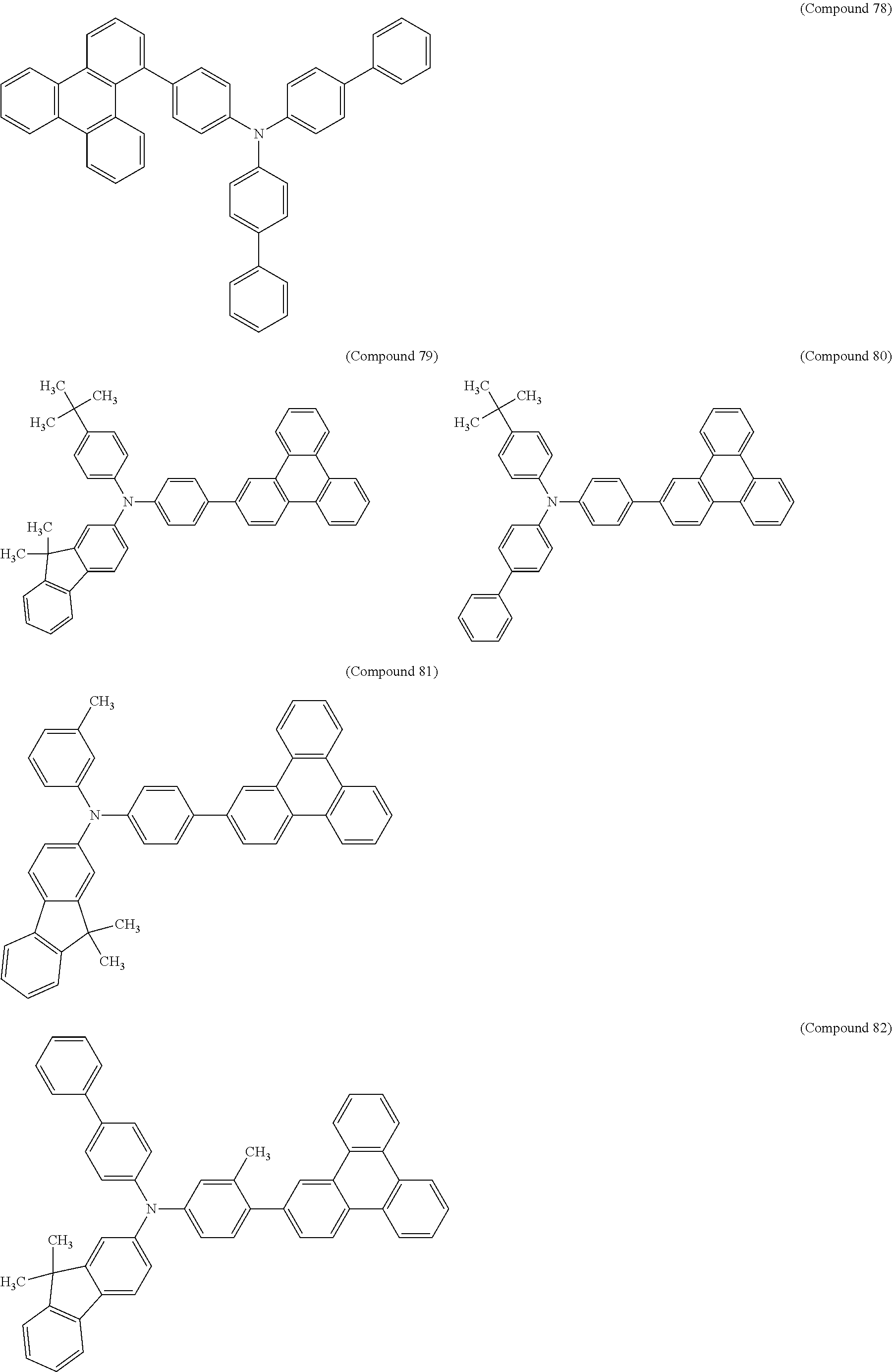

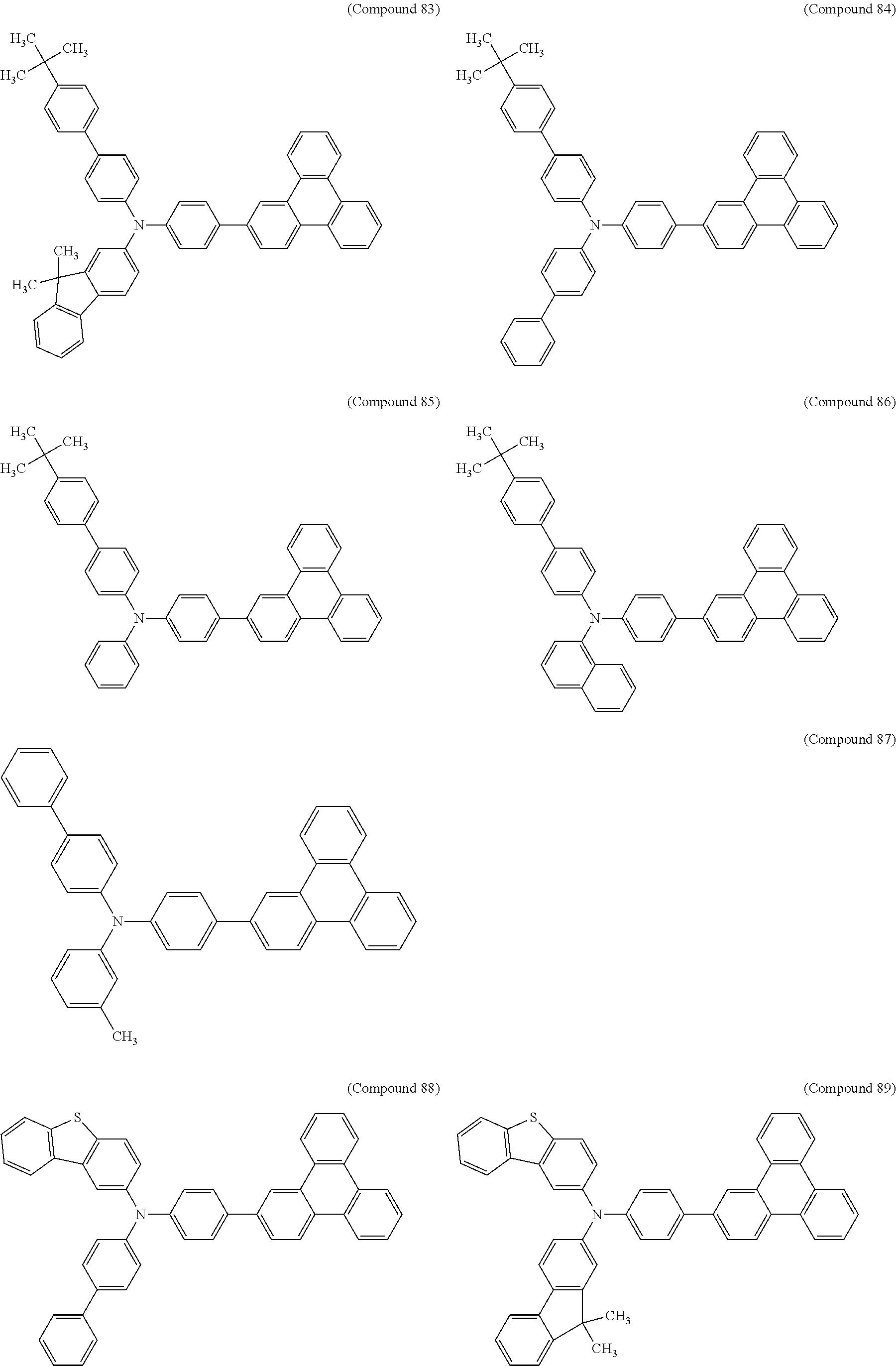

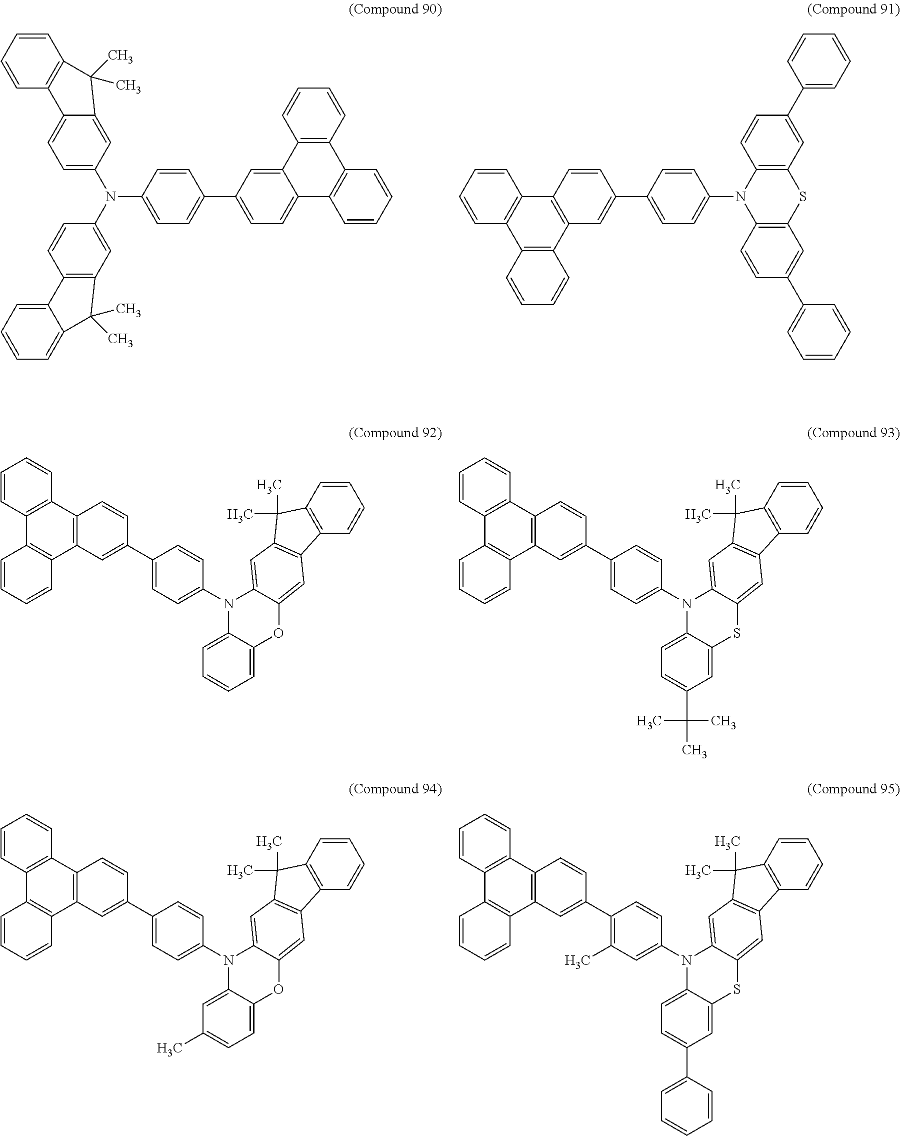

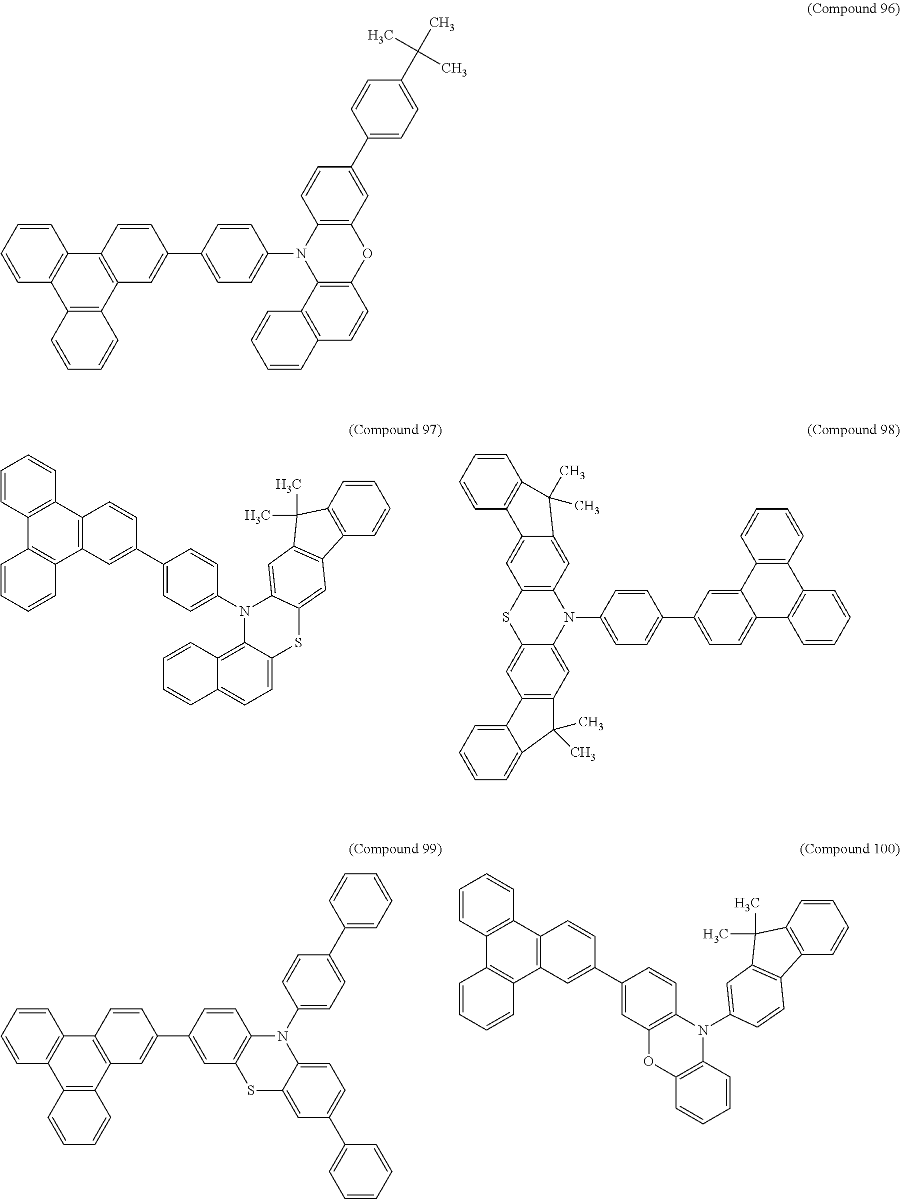

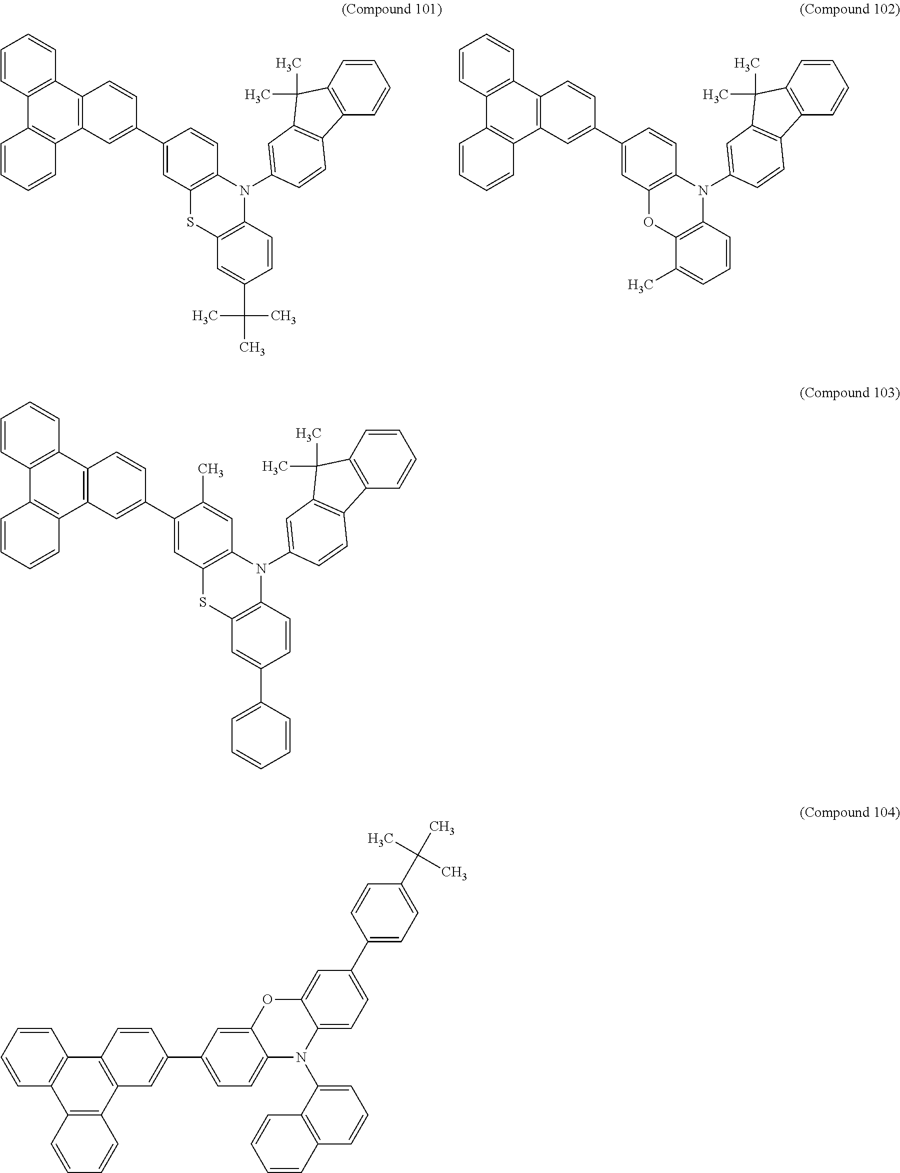

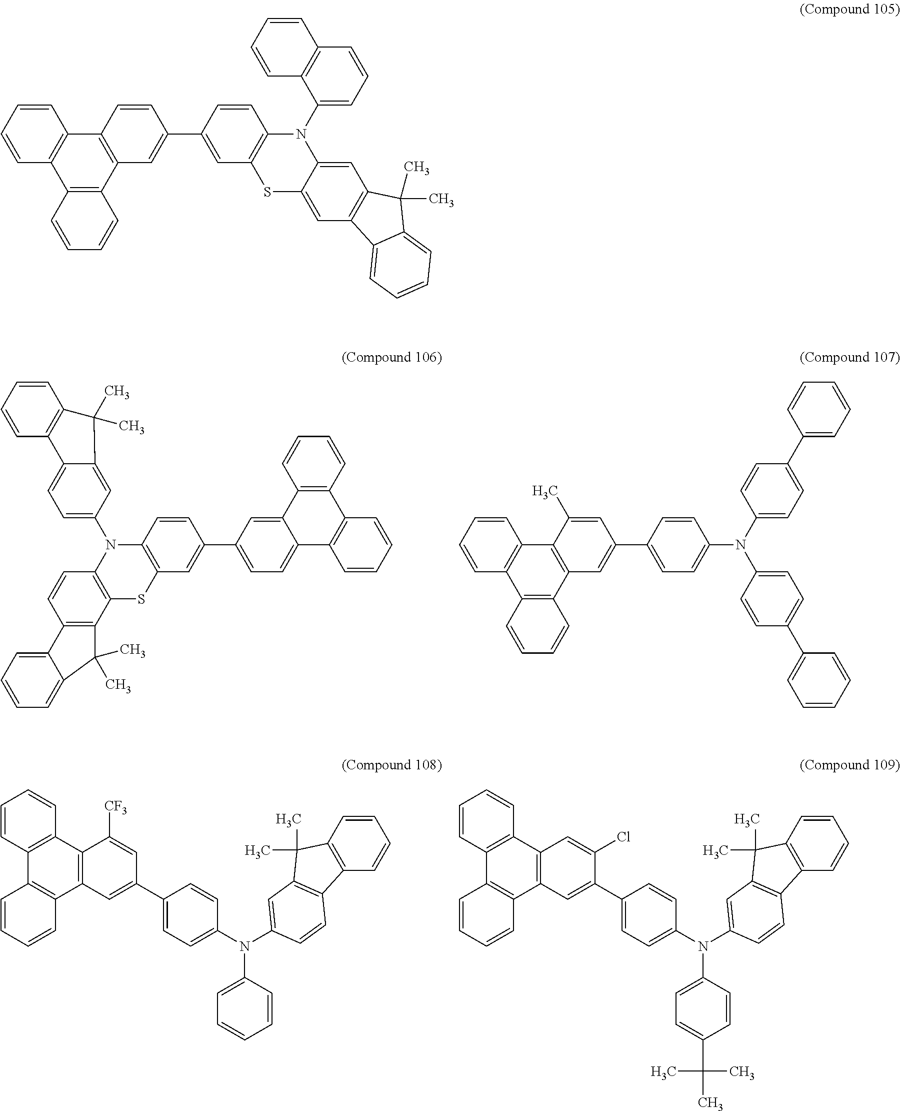

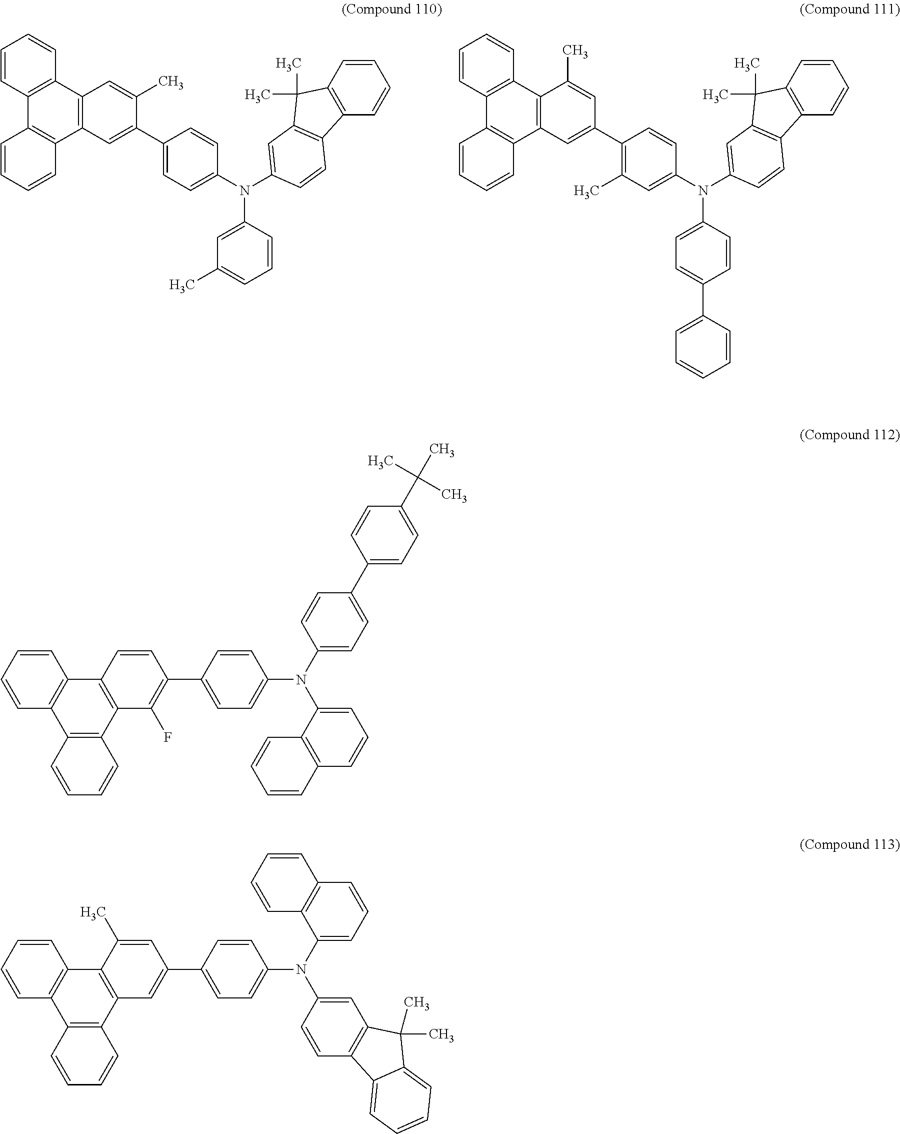

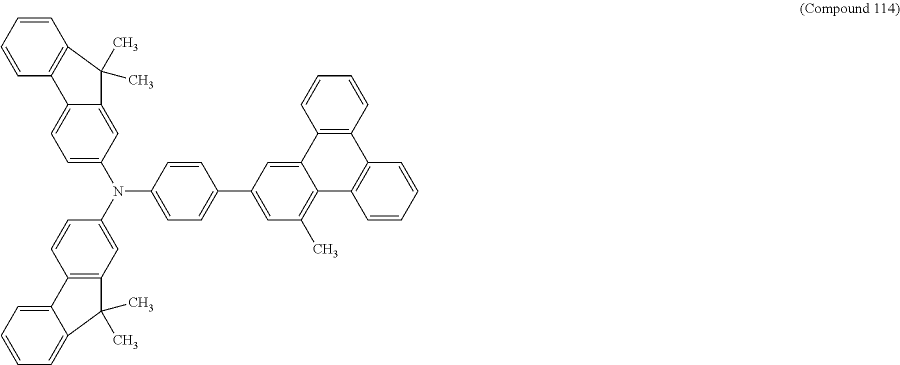

As compared to the conventional known hole-transporting materials, the above-mentioned triphenylene derivative of the invention has a high glass transition point (Tg), is capable of forming a thin film excellent in heat resistance, maintains an amorphous state with stability, and is capable of maintaining stability in a thin-film form. Moreover, the triphenylene derivative of the invention features good electron-blocking power. For example, a film formed by vapor-depositing the triphenylene derivative of the invention in a thickness of 100 nm can be measured for its work function to exhibit a very high value.

Therefore, the triphenylene derivative of the invention is very useful as a material for forming an organic layer of an organic EL device.

### <Organic EL Device>

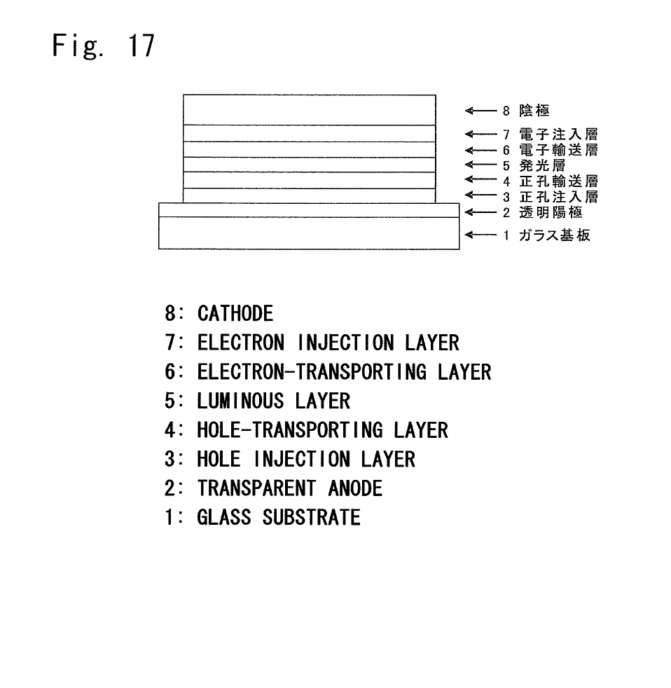

The organic EL device having the organic layer formed by using the above triphenylene derivative of the present invention has a structure of layers as shown, for example, in FIG. 17.

Namely, a transparent anode 2, a hole injection layer 3, a hole-transporting layer 4, a luminous layer 5, an electron-transporting layer 6, an electron injection layer 7 and a cathode 8 are formed on a glass substrate 1 (which may be any transparent substrate such as transparent resin substrate or the like substrate).

The organic EL device to which the triphenylene derivative of the present invention is applied is not limited to the one of the above layer structure, as a matter of course. For instance, the organic EL device may have an electron-blocking layer formed between the hole-transporting layer 4 and the luminous layer 5, may have a hole-blocking layer formed between the luminous layer 5 and the hole-transporting layer 6, or may have a simplified layer structure omitting the electron injection layer 7 and the hole injection layer 3. For instance, some layers can be omitted from the above multilayer structure. Namely, the organic EL device can be fabricated in a simple layer structure having the anode 2, hole-transporting layer 4, luminous layer 5, electron-transporting layer 6 and cathode 8 formed on the substrate 1.

That is, the triphenylene derivative of the invention is preferably used as a material for forming organic layers (e.g., hole injection layer 3, hole-transporting layer 4, electron-blocking layer that is not shown, and luminous layer 5) between the anode 2 and the cathode 8.

In the organic EL device, the transparent anode 2 may be formed by using an electrode material which has been known per se, i.e., by vapor-depositing an electrode material having a large work function, such as ITO or gold on the substrate 1 (transparent substrate such as glass substrate).

Further, the hole injection layer 3 can be formed on the transparent electrode 2 by using the triphenylene derivative of the present invention and by using the materials that have been known per se, such as those described below.
Porphyrin compound as represented by copper phthalocyanine;
Triphenylamine derivative of the star burst type;
Arylamine (e.g., trimer or tetramer of triphenylamine) having a structure in which a plurality of triphenylamine skeletons are coupled together via a single bond or a divalent group without hetero atom;
High molecular materials of the application type, such as poly(3,4-ethylenedioxythiophene) (PEDOT), poly(styrene sulfonate) (PSS), etc.; and
Acceptor-type heterocyclic compounds such as hexacyanoazatriphenylene and the like.

The layer (thin film) can be formed by using the above materials relying not only upon the vacuum evaporation method but also upon the known methods such as spin-coating method or ink-jet method. The layers described below, too, can similarly be formed by the vacuum evaporation, the spin-coating or the ink-jet method.

The hole-transporting layer 4, too, can be formed on the hole injection layer 3 by using the above-mentioned triphenylene derivative of the present invention or by using a hole-transporting material that has been known per se.

Representative examples of the hole materials that have been known per se. are:

Benzidine derivatives such as,
N,N′-Diphenyl-N,N′-di(m-tolyl)benzidine (hereinafter abbreviated as TPD);
N,N′-Diphenyl-N,N′-di(α-naphthyl)benzidine (hereinafter abbreviated as NPD); and
N,N,N′,N′-Tetrabiphenylylbenzidine;
   Amine derivatives such as,
1,1-Bis[4-(di-4-tolylamino)phenyl]cyclohexane (hereinafter abbreviated as TAPC);
Various kinds of triphenylamine trimers and tetramers; and
The above-mentioned application-type high molecular materials that can also be used for forming the hole injection layer.

The compounds which are the hole-transporting materials may be used alone to form a film or may be used being mixed together in two or more kinds to form a film. Or the above compounds may be used in one kind or in a plurality of kinds to form a plurality of layers, and a multiplicity of films formed by laminating such layers may be used as a hole-transporting layer.

It is, further, allowable to form a layer which works both as the hole injection layer 3 and the hole-transporting layer 4. Such a hole injection/transporting layer can be formed by applying a high molecular material such as poly(3,4-ethylenedioxythiophene (hereinafter abbreviated as PEDOT)).

In forming the hole-transporting layer 4 (the same holds for the hole injection layer 3, too), the material usually used for forming the layer may, further, be P-doped with a trisbromophenylaminehexachloroantimony or the like. It is also allowable to form the hole-transporting layer 4 (or the hole injection layer 3) by using a high molecular compound having a basic skeleton of TPD.

Further, as the electron-blocking layer (that can be formed between the luminous layer 5 and the hole-transporting layer 4) that has not been shown, there can be used the triphenylene derivative that has the electron-blocking action of the present invention as well as a known compound having the electron-blocking action, such as carbazole derivative or a compound that has a triphenylsilyl group yet having a triarylamine structure. Described below are concrete examples of the carbazole derivative and the compound having the triarylamine structure.

### <Carbazole Derivatives>

4,4′,4″-Tri(N-carbazolyl)triphenylamine (hereinafter abbreviated as TCTA);
9,9-Bis[4-(carbazole-9-yl)phenyl]fluorene;
1,3-Bis(carbazole-9-yl)benzene (hereinafter abbreviated as mCP); and
2,2-Bis(4-carbazole-9-ylphenyl)adamantine (hereinafter abbreviated as Ad-Cz).

### <Compounds Having a Triarylamine Structure>

9-[4-(Carbazole-9-yl)phenyl]-9-[4-(triphenylsilyl) phenyl]-9H-fluorene.

The electron-blocking layer is formed by using one, two or more kinds of the triphenylene compounds of the invention or the above known hole-transporting materials. It is, however, also allowable to form a plurality of layers by using one or a plurality of kinds of the hole-transporting materials, and use a multiplicity of films formed by laminating such layers as the electron-blocking layer.

The luminous layer 5 of the organic EL device can be formed by using a metal complex of a quinolynol derivative as represented by Alq₃ as well as various metal complexes such as of zinc, beryllium and aluminum, and luminous materials such as anthracene derivative, bisstyrylbenzene derivative, pyrene derivative, oxazole derivative and polyparaphenylenevinylene derivative.

It is also allowable to constitute the luminous layer 5 by using a host material and a dopant material.

As the host material in this case, there can be used thiazole derivative, benzimidazole derivative and polydialkylfluorene derivative in addition to the above luminous materials, as well as the above-mentioned triphenylene derivative of the present invention.

As the dopant material, there can be used quinacridone, cumalin, rubrene, perylene and derivatives thereof, benzopyran derivative, Rhodamine derivative and aminostyryl derivative.

The luminous layer 5, too, can be formed in a single-layer structure by using one or two or more kinds of the luminous materials, or in a multi-layer structure by laminating a plurality of layers.

It is, further, allowable to form the luminous layer 5 by using a phosphorescent luminous material as the luminous material.

As the phosphorescent luminous material, there can be used a phosphorescent luminous body of a metal complex such as of iridium or platinum. For example, there can be used a green luminous phosphor such as Ir(ppy)₃, a blue luminous phosphor such as Flrpic or Flr₆, and a red luminous phosphor such as Btp₂lr(acac). These phosphorescent luminous materials are used by being added to the hole injection/transporting host material or the electron-transporting host material.

As the hole injection/transporting host material, there can be used the triphenylene derivative of the present invention as well as 4,4′-di(N-carbazolyl)biphenyl (hereinafter abbreviated as CBP), or carbazole derivative such as TCTA or mCP.

As the electron-transporting host material, there can be used p-bis(triphenylsilyl)benzene (hereinafter abbreviated as UGH2) or 2,2′,2″-(1,3,5-phenylene)-tris(1-phenyl-1H-benzimidazole) (hereinafter abbreviated as TPBI).

To avoid the concentration quenching, the host material is desirably doped with the phosphorescent luminous material in an amount in a range of 1 to 30% by weight relative to the whole luminous layer relying on the vacuum coevaporation.

The hole-blocking layer (not shown in FIG. 17) can be formed between the luminous layer 5 and the electron-transporting layer 6 by using a compound having hole-blocking action that has been known per se.

As the known compound having the hole-blocking action, there can be exemplified phenanthroline derivatives such as bathocuproin (hereinafter abbreviated as BCP), metal complexes of quinolinol derivatives such as aluminum (III) bis(2-methyl-8-quinolinato)-4-phenylphenolate (hereinafter abbreviated as BAlq) and the like, as well as triazole derivatives, triazine derivatives and oxadiazole derivatives.

These materials can also be used for forming the electron-transporting layer 6 that will be described below. Moreover, the hole-blocking layer and the electron-transporting layer 6 can be formed as one layer.

The hole-blocking layer, too, can be formed in the structure of a single layer or of a laminate of a multiplicity of layers, the layers being formed by using one kind, two kinds or more kinds of the above-mentioned compounds having hole-blocking action.

The electron-transporting layer 6 can be formed by using electron-transporting compounds that have been known per se. such as metal complexes of quinolinol derivatives like Alqₐ, BAlq, as well as various metal complexes such as of zinc, beryllium and aluminum, triazole derivatives, triazine derivatives, oxadiazole derivatives, thiadiazole derivatives, carbodiimide derivatives, quinoxaline derivatives, phenanthroline derivatives and silole derivatives.

The electron-transporting layer 6, too, can be formed in the structure of a single layer or of a laminate of a multiplicity of layers, the layers being formed by using one kind, two kinds or more kinds of the above-mentioned electron-transporting compounds.

The electron injection layer 7, too, can be formed by using known compounds, i.e., by using alkali metal salts such as lithium fluoride and cesium fluoride, alkaline earth metal salts such as magnesium fluoride, and metal oxides such as aluminum oxide.

As the cathode 8 of the organic EL device, there can be used an electrode material having a low work function, such as aluminum, or an electrode material of an alloy having a lower work function, such as magnesium-silver alloy, magnesium-indium alloy or aluminum-magnesium alloy.

The organic EL device forming at least one of the organic layers (e.g., at least any one of electron injection layer 3, electron-transporting layer 4, hole-blocking layer or luminous layer 5) by using the triphenylene derivative of the present invention, features a high luminous efficiency, a high power efficiency, a low practical driving voltage, a low luminance start voltage and very excellent durability.

### EXAMPLES

The invention will now be concretely described by way of Examples to which only, however, the invention is in no way limited.

### Example 1

### Synthesis of a bis(biphenyl-4-yl)-{4-(triphenylene-2-yl) phenyl}amine

### Synthesis of a Compound 66

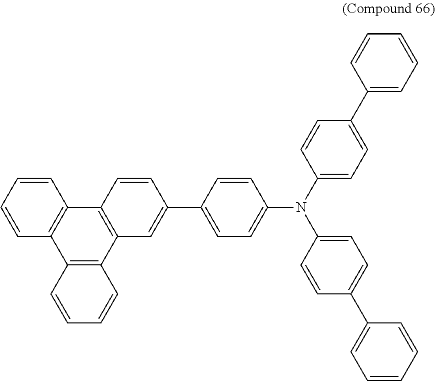 [SMILES: c1ccc(-c2ccc(N(c3ccc(-c4ccccc4)cc3)c3ccc(-c4ccc5c6ccccc6c6ccccc6c5c4)cc3)cc2)cc1]

| | | |
|---|---|---|
| Bis(biphenyl-4-yl)-(4-bromophenyl)amine | 3.85 | g, |
| 4,4,5,5-Tetramethyl-2-(triphenylene-2-yl)- | 2.83 | g, |
| [1,3,2]dioxaborane | | |
| Toluene | 59 | ml, |
| Ethanol | 15 | ml, and |
| 2M Potassium carbonate aqueous solution | 6 | ml, |

were put into a reaction vessel in a nitrogen atmosphere, and to which a nitrogen gas was flown for 30 minutes while being irradiated with ultrasonic waves.

Next, 0.19 g of a tetrakis(triphenylphosphine) palladium was added thereto, and the mixture was heated and stirred at 72° C. for 4.5 hours. After left to cool down to room temperature, 50 ml of methanol was added thereto, and the precipitated crude product was picked up by filtration.

The crude product was dissolved in 300 ml of toluene, refined by adsorption by using 7.5 g of silica gel, concentrated under reduced pressure and, thereafter, the crystals thereof were precipitated by using a mixed solvent of 1,2-dichlorobenzene and toluene. Upon conducting the reflux washing with methanol, there was obtained 3.30 g of a white powder of bis(biphenyl-4-yl)-{4-(triphenylene-2-yl)phenyl}amine (compound 66) (yield, 66%).

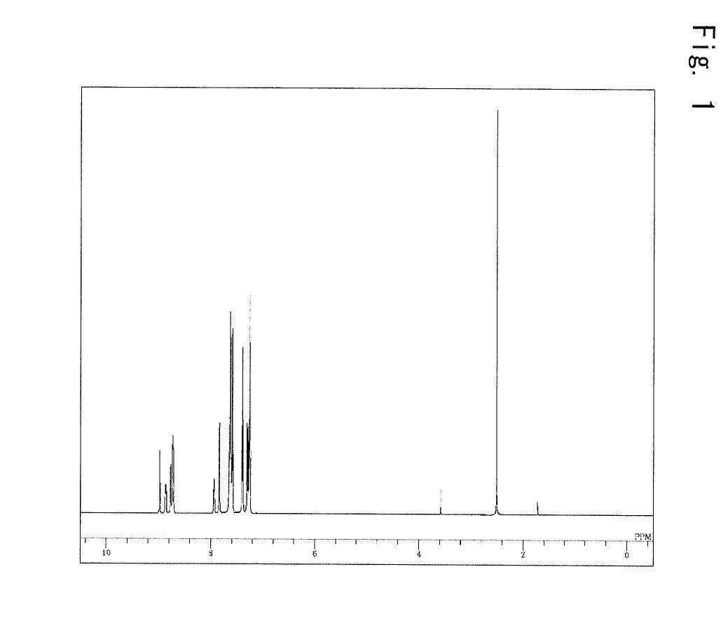

The obtained white powder was identified for its structure by the NMR. FIG. 1 shows the results of the ¹H-NMR measurement.

The following 33 signals of hydrogen were detected by the ¹H-NMR (THF-d₈).

δ (ppm)=
8.98 (1H)
8.87 (1H)
8.78-8.71 (4H)
7.94 (1H)
7.84 (2H)
7.65-7.59 (12H)
7.39 (4H)
7.32-7.22 (8H)

### Example 2

### Synthesis of a (9,9-dimethyl-9H-fluorene-2-yl)-phenyl-{4-(triphenylene-2-yl)phenyl}amine

### Synthesis of a Compound 15

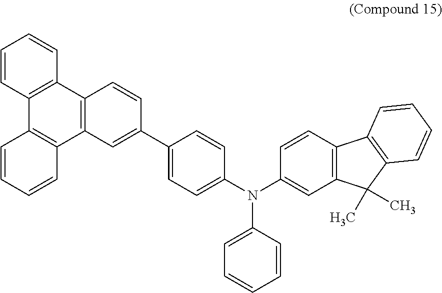 [SMILES: CC1(C)c2ccccc2-c2ccc(N(c3ccccc3)c3ccc(-c4ccc5c6ccccc6c6ccccc6c5c4)cc3)cc21]

| | | |
|---|---|---|
| 4-Bromophenyl(9,9-dimethyl-9H-fluorene-2-yl)- | 3.89 | g, |
| phenylamine | | |
| 4,4,5,5-Tetramethyl-2-(triphenylene-2-yl)- | 3.08 | g, |
| [1,3,2]dioxaborane | | |
| Toluene | 59 | ml, |
| Ethanol | 15 | ml, and |
| 2M Potassium carbonate aqueous solution | 6.5 | ml, |

were put into the reaction vessel in the nitrogen atmosphere, and to which the nitrogen gas was flown for 30 minutes while being irradiated with ultrasonic waves.

Next, 0.21 g of the tetrakis(triphenylphosphine) palladium was added thereto, and the mixture was heated and stirred at 72° C. for 5.5 hours. After left to cool down to room temperature, 50 ml of water and 30 ml of toluene were added thereto, and the organic layer was picked up by the separating operation. The organic layer was dried on the anhydrous magnesium sulfate and was, thereafter, concentrated under reduced pressure to obtain a brown crude product.

The crude product was dissolved in 250 ml of toluene, refined by adsorption by using 7.5 g of silica gel, concentrated under reduced pressure and, further, refined by the column chromatography (carrier: silica gel, eluent: hexane/toluene), and the crystals were precipitated by using a mixed solvent of toluene and methanol. Upon reflux-washing the crystals with methanol, there was obtained 3.34 g of a white powder of (9,9-dimethyl-9H-fluorene-2-yl)-phenyl-{4-(triphenylene-2-yl)phenyl}amine (compound 15) (yield, 65%).

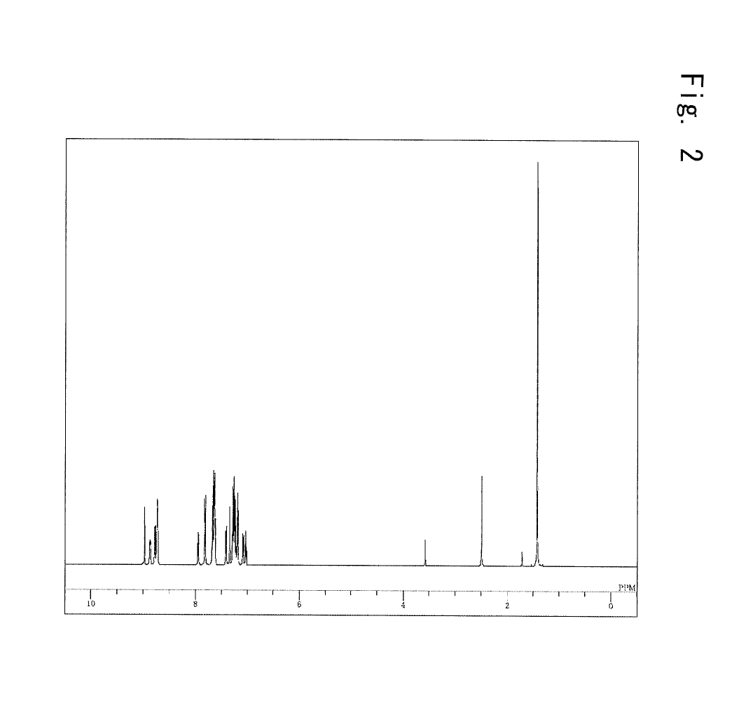

The obtained white powder was identified for its structure by the NMR. FIG. 2 shows the results of the ¹H-NMR measurement.

The following 33 signals of hydrogen were detected by the ¹H-NMR (THF-d₈).

δ (ppm)=
8.98 (1H)
8.88 (1H)
8.79-8.73 (4H)
7.95 (1H)
7.82 (2H)
7.69-7.62 (6H)
7.41 (1H)
7.35 (1H)
7.30-7.19 (8H)
7.09 (1H)
7.04 (1H)
1.43 (6H)

### Example 3

### Synthesis of a (biphenyl-4-yl)-(9,9-dimethyl-9H-fluorene-2-yl)-{4-(triphenylene-2-yl)phenyl}amine

### Synthesis of a Compound 67

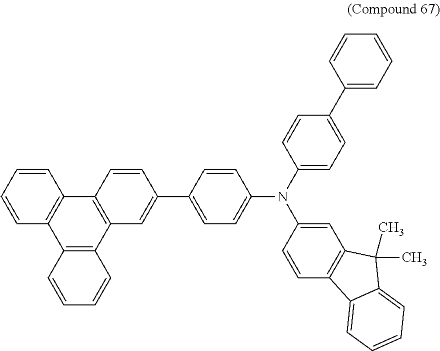 [SMILES: CC1(C)c2ccccc2-c2ccc(N(c3ccc(-c4ccccc4)cc3)c3ccc(-c4ccc5c6ccccc6c6ccccc6c5c4)cc3)cc21]

| | | |
|---|---|---|
| (Biphenyl-4-yl)-(9,9-dimethyl-9H- | 17.9 | g, |
| fluorene-2-yl) Amine | | |
| 2-(4-Bromophenyl)triphenylene | 19.0 | g, |
| Tert-butoxysodium | 5.72 | g, and |
| Toluene | 200 | ml, |

were put into the reaction vessel in the nitrogen atmosphere, and to which the nitrogen gas was flown for 30 minutes while being irradiated with ultrasonic waves.

Next,

| | |
|---|---|
| Palladium acetate | 0.22 g and |
| Toluene solution of tris-tert-butylphosphine | 1.9 ml, |
| (50% w/v) | |

were added thereto, and the mixture was heated and stirred at 80° C. for 1.5 hours. After left to cool down to room temperature, 100 ml of water and 100 ml of toluene were added thereto, and the organic layer was picked up by the separating operation. The organic layer was dried on the anhydrous magnesium sulfate and was, thereafter, concentrated under reduced pressure to obtain a brown crude product.

The crude product was dissolved in 750 ml of toluene, refined by adsorption by using 30 g of silica gel, crystallized by using a mixed solvent of toluene and hexane followed by the reflux-washing with methanol to obtain 28.2 g of a white powder of (biphenyl-4-yl)-(9,9-dimethyl-9H-fluorene-2-yl)-{4-(triphenylene-2-yl)phenyl}amine (compound 67) (yield, 83%).

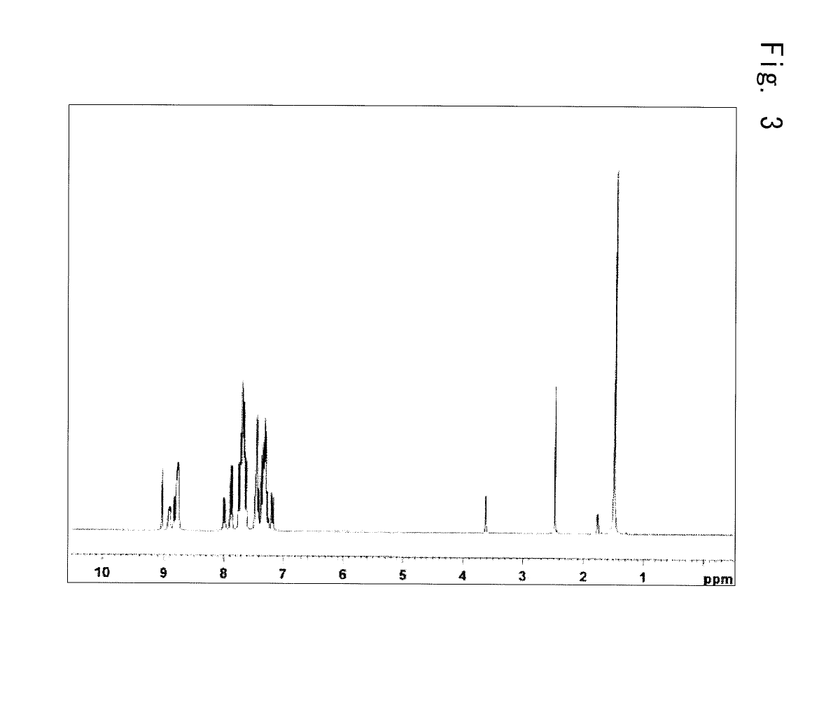

The obtained white powder was identified for its structure by the NMR. FIG. 3 shows the results of the ¹H-NMR measurement.

The following 37 signals of hydrogen were detected by the ¹H-NMR (THF-d₈).

δ (ppm)=
8.98 (1H)
8.88 (1H)
8.79-8.73 (4H)
7.95 (1H)
7.87 (2H)
7.75-7.62 (10H)
7.44 (4H)
7.37-7.28 (7H)
7.18 (1H)
1.49 (6H)

### Example 4

### Synthesis of a (4-tert-butylphenyl)-(9,9-dimethyl-9H-fluorene-2-yl)-{4-(triphenylene-2-yl)phenyl}amine

### Synthesis of a Compound 79

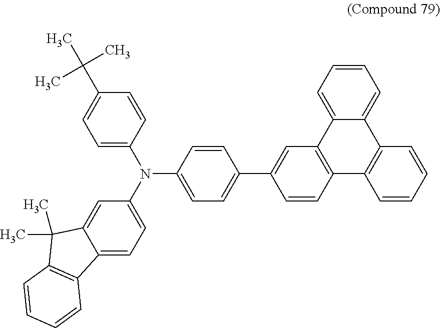 [SMILES: CC(C)(C)c1ccc(N(c2ccc(-c3ccc4c5ccccc5c5ccccc5c4c3)cc2)c2ccc3c(c2)C(C)(C)c2ccccc2-3)cc1]

| | | |
|---|---|---|
| (4-Bromophenyl)-(4-tert-butylphenyl)-(9,9- | 15.4 | g, |
| dimethyl-9H-fluorene-2-yl)amine | | |
| 4,4,5,5-Tetramethyl-2-(triphenylene-2-yl)- | 11.0 | g, |
| [1,3,2]dioxaborane | | |
| Toluene | 88 | ml, |
| Ethanol | 22 | ml, and |
| 2M Potassium carbonate aqueous solution | 31 | ml, |

were put into the reaction vessel in the nitrogen atmosphere, and to which the nitrogen gas was flown for 30 minutes while being irradiated with ultrasonic waves.

Next, 0.62 g of the tetrakis(triphenylphosphine) palladium was added thereto, and the mixture was heated and stirred at 72° C. for 3 hours. After left to cool down to room temperature, 50 ml of water and 100 ml of toluene were added thereto, and the organic layer was picked up by the separating operation. The organic layer was dried on the anhydrous magnesium sulfate and was, thereafter, concentrated under reduced pressure to obtain an orange crude product.

The crude product was refined by the column chromatography (carrier: silica gel, eluent: hexane/toluene), crystallized by using a mixed solvent of toluene and hexane, and was reflux-washed with methanol to obtain 14.5 g of a white powder of (4-tert-butylphenyl)-(9,9-dimethyl-9H-fluorene-2-yl)-{4-(triphenylene-2-yl)phenyl}amine (compound 79) (yield, 73%).

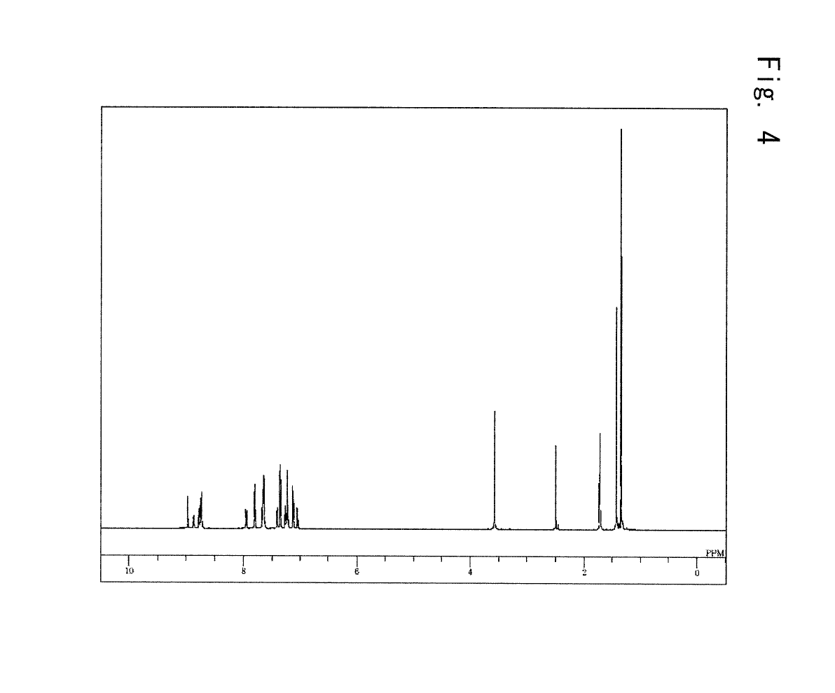

The obtained white powder was identified for its structure by the NMR. FIG. 4 shows the results of the ¹H-NMR measurement.

The following 41 signals of hydrogen were detected by the ¹H-NMR (THF-d₈).

δ (ppm)=
8.98 (1H)
8.87 (1H)
8.77 (4H)
7.95 (1H)
7.81 (2H)
7.68-7.64 (6H)
7.41 (1H)
7.36 (3H)
7.27-7.22 (4H)
7.13 (2H)
7.06 (1H)
1.44 (6H)
1.35 (9H)

### Example 5

### Synthesis of a (biphenyl-4-yl)-(4-tert-butylphenyl)-{4-(triphenylene-2-yl)phenyl}amine

### Synthesis of a Compound 80

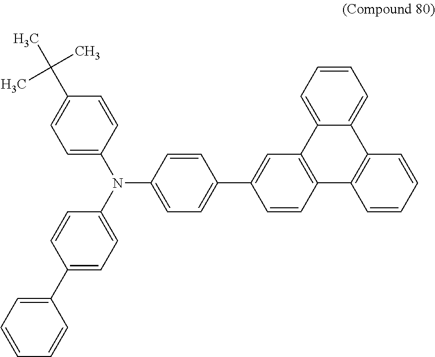 [SMILES: CC(C)(C)c1ccc(N(c2ccc(-c3ccccc3)cc2)c2ccc(-c3ccc4c5ccccc5c5ccccc5c4c3)cc2)cc1]

| | | |
|---|---|---|
| (Biphenyl-4-yl)-(4-bromophenyl)- | 15.1 | g, |
| (4-tert-butylphenyl)amine | | |
| 4,4,5,5-Tetramethyl-2-(triphenylene-2-yl)- | 11.7 | g, |
| [1,3,2]dioxaborane | | |
| Toluene | 96 | ml, |
| Ethanol | 24 | ml, and |
| 2M Potassium carbonate aqueous solution | 33 | ml, |

were put into the reaction vessel in the nitrogen atmosphere, and to which the nitrogen gas was flown for 30 minutes while being irradiated with ultrasonic waves.

Next, 0.77 g of the tetrakis(triphenylphosphine) palladium was added thereto, and the mixture was heated and stirred at 72° C. for 4 hours. After left to cool down to room temperature, 100 ml of water and 150 ml of toluene were added thereto, and the organic layer was picked up by the separating operation. The organic layer was dried on the anhydrous magnesium sulfate and was, thereafter, concentrated under reduced pressure to obtain a grey crude product.

The crude product was dissolved in 300 ml of toluene, refined by adsorption by using 20 g of silica gel, crystallized by using the mixed solvent of toluene and hexane, crystallized again by using the mixed solvent of toluene and methanol, and was, further, reflux-washed with methanol to obtain 16.7 g of a white powder of (biphenyl-4-yl)-(4-tert-butylphenyl)-{4-(triphenylene-2-yl)phenyl}amine (compound 80) (yield, 83%).

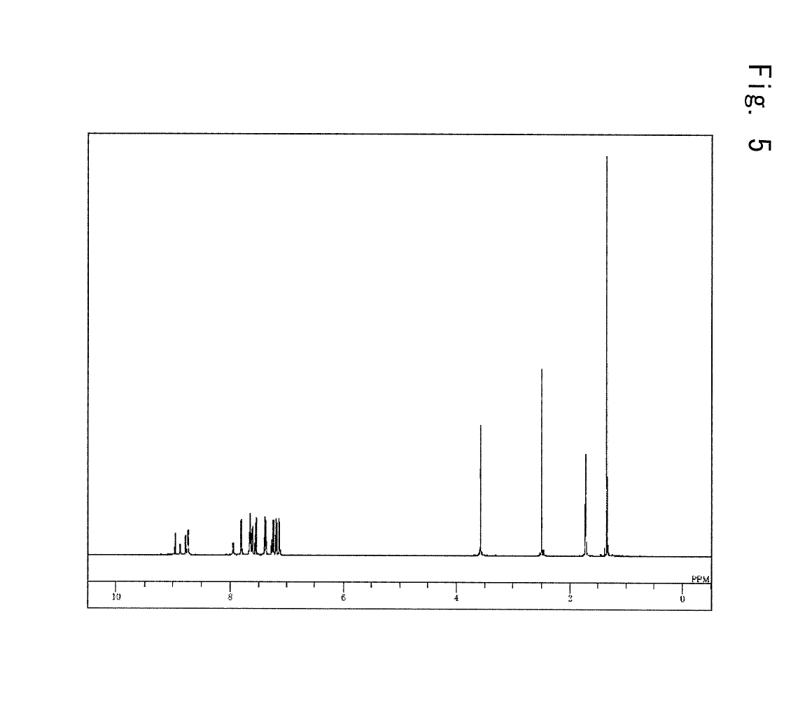

The obtained white powder was identified for its structure by the NMR. FIG. 5 shows the results of the ¹H-NMR measurement.

The following 37 signals of hydrogen were detected by the ¹H-NMR (THF-d₈).

δ (ppm)=
8.97 (1H)
8.87 (1H)
8.77 (4H)
7.95 (1H)
7.81 (2H)
7.64 (6H)
7.56 (2H)
7.41-7.37 (4H)
7.26 (3H)
7.19 (2H)
7.14 (2H)
1.34 (9H)

### Example 6

### Synthesis of a (9,9-dimethyl-9H-fluorene-2-yl)-(3-methylphenyl)-{4-(triphenylene-2-yl)phenyl}amine

### Synthesis of a Compound 81

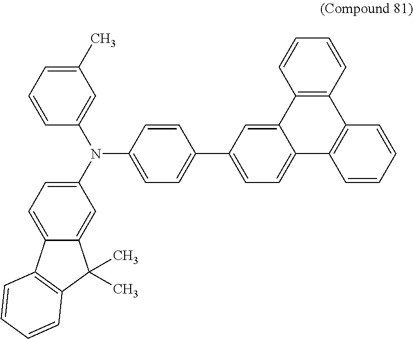 [SMILES: Cc1cccc(N(c2ccc(-c3ccc4c5ccccc5c5ccccc5c4c3)cc2)c2ccc3c(c2)C(C)(C)c2ccccc2-3)c1]

| | | |
|---|---|---|
| (4-Bromophenyl)-(9,9-dimethyl-9H-fluorene- | 15.0 | g, |
| 2-yl)-(3-methylphenyl)amine | | |
| 4,4,5,5-Tetramethyl-2-(triphenylene-2-yl)- | 12.3 | g, |
| [1,3,2]dioxaborane | | |
| Toluene | 120 | ml, |
| Ethanol | 30 | ml, and |
| 2M Potassium carbonate aqueous solution | 33 | ml, |

were put into the reaction vessel in the nitrogen atmosphere, and to which the nitrogen gas was flown for 30 minutes while being irradiated with ultrasonic waves.

Next, 0.76 g of the tetrakis(triphenylphosphine) palladium was added thereto, and the mixture was heated and stirred at 72° C. for 20.5 hours. After left to cool down to room temperature, 50 ml of water and 50 ml of toluene were added thereto, and the organic layer was picked up by the separating operation. The organic layer was dried on the anhydrous magnesium sulfate and was, thereafter, concentrated under reduced pressure to obtain a brown crude product.

The crude product was refined by the column chromatography (carrier: silica gel, eluent: hexane/toluene), crystallized by using the mixed solvent of toluene and methanol, crystallized again by using the mixed solvent of toluene and hexane, and was, further, reflux-washed with methanol to obtain 13.1 g of a white powder of (9,9-dimethyl-9H-fluorene-2-yl)-(3-methylphenyl)-{4-(triphenylene-2-yl)phenyl}amine (compound 81) (yield, 66%).

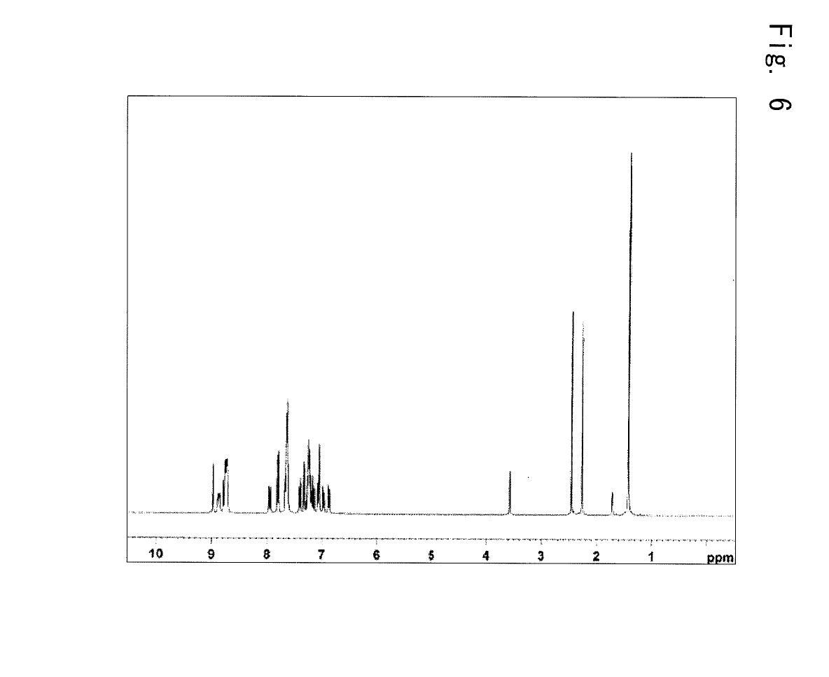

The obtained white powder was identified for its structure by the NMR. FIG. 6 shows the results of the ¹H-NMR measurement.

The following 35 signals of hydrogen were detected by the ¹H-NMR (THF-d₈).

δ (ppm)=
8.98 (1H)
8.88 (1H)
8.79-8.73 (4H)
7.95 (1H)
7.80 (2H)
7.68-7.62 (6H)
7.41 (1H)
7.33-7.14 (6H)
7.06 (2H)
6.97 (1H)
6.87 (1H)
2.27 (3H)
1.43 (6H)

### Example 7

### Synthesis of a (biphenyl-4-yl)-(9,9-dimethyl-9H-fluorene-2-yl)-(3-methyl-4-(triphenylene-2-il)phenyl}amine

### Synthesis of a Compound 82

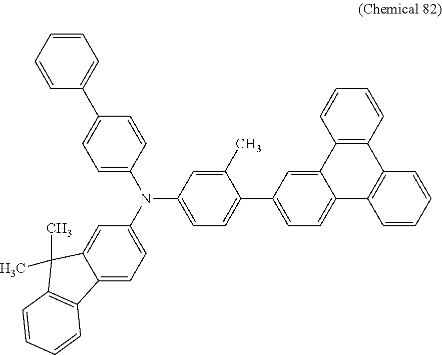 [SMILES: Cc1cc(N(c2ccc(-c3ccccc3)cc2)c2ccc3c(c2)C(C)(C)c2ccccc2-3)ccc1-c1ccc2c3ccccc3c3ccccc3c2c1]

| | | |
|---|---|---|
| (Biphenyl-4-yl)-(4-bromo-3-methylphenyl)- | 17.0 | g, |
| (9,9-dimethyl-9H-fluorene-2-yl)amine | | |
| 4,4,5,5-Tetramethyl-2-(triphenylene-2-yl)- | 11.4 | g, |
| [1,3,2]dioxaborane | | |
| Toluene | 136 | ml, |
| Ethanol | 34 | ml, and |
| 2M Potassium carbonate aqueous solution | 32 | ml, |

were put into the reaction vessel in the nitrogen atmosphere, and to which the nitrogen gas was flown for 30 minutes while being irradiated with ultrasonic waves.

Next, 0.74 g of the tetrakis(triphenylphosphine) palladium was added thereto, and the mixture was heated and stirred at 72° C. for 6 hours. After left to cool down to room temperature, 100 ml of water and 100 ml of toluene were added thereto, and the organic layer was picked up by the separating operation. The organic layer was dried on the anhydrous magnesium sulfate and was, thereafter, concentrated under reduced pressure to obtain a brown crude product.

The crude product was refined by the column chromatography (carrier: silica gel, eluent: hexane/toluene), crystallized by using the mixed solvent of tetrahydrofurane and methanol, and was, further, reflux-washed with methanol to obtain 13.1 g of a faintly yellow powder of (biphenyl-4-yl)-(9,9-dimethyl-9H-fluorene-2-yl)-{3-methyl-4-(triphenylene-2-yl)phenyl}amine (compound 82) (yield, 66%).

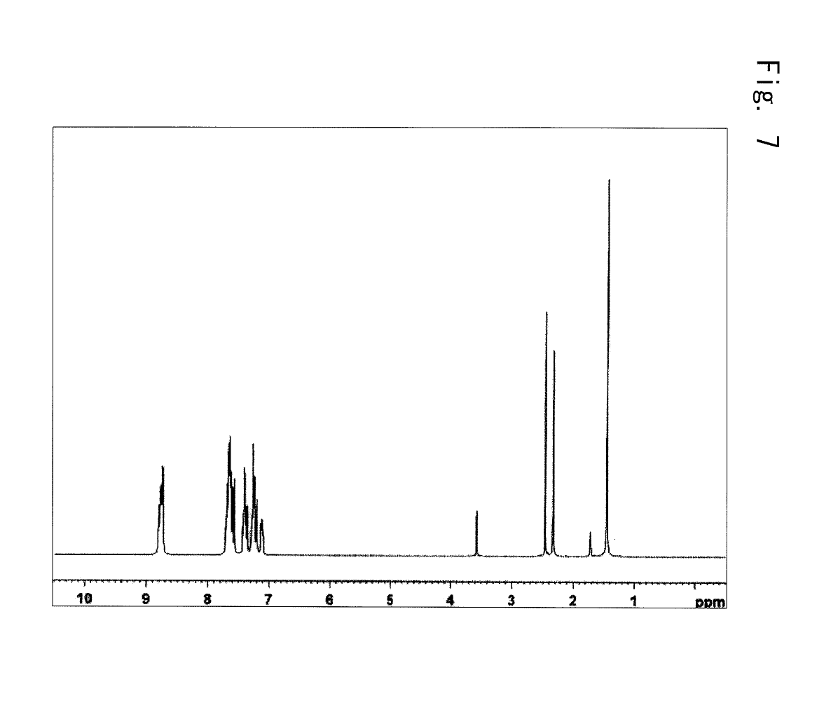

The obtained faintly yellow powder was identified for its structure by the NMR. FIG. 7 shows the results of the ¹H-NMR measurement.

The following 39 signals of hydrogen were detected by the ¹H-NMR (THF-d₈).

δ (ppm)=
8.98-8.73 (6H)
7.66-7.67 (11H)
7.43-7.35 (5H)
7.28-7.20 (6H)
7.14-7.09 (2H)
2.34 (3H)
1.43 (6H)

### Example 8

### Synthesis of a (4′-tert-butylbiphenyl-4-yl)-(9,9-dimethyl-9H-fluorene-2-yl)-{4-(triphenylene-2-yl)phenyl}amine

### Synthesis of a Compound 83

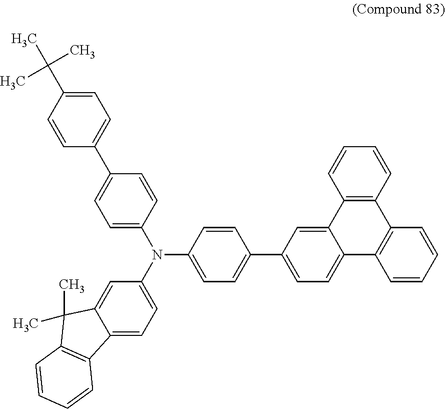 [SMILES: CC(C)(C)c1ccc(-c2ccc(N(c3ccc(-c4ccc5c6ccccc6c6ccccc6c5c4)cc3)c3ccc4c(c3)C(C)(C)c3ccccc3-4)cc2)cc1]

| | | |
|---|---|---|
| (4-Bromophenyl)-(4′-tert-butylbiphenyl-4-yl)- | 17.5 | g, |
| (9,9-dimethyl-9H-fluorene-2-yl)amine | | |
| 4,4,5,5-Tetramethyl-2-(triphenylene-2-yl)- | 8.9 | g, |
| [1,3,2]dioxaborane | | |
| Toluene | 314 | ml, |
| Ethanol | 79 | ml, and |
| 2M Potassium carbonate aqueous solution | 19 | ml, |

were put into the reaction vessel in the nitrogen atmosphere, and to which the nitrogen gas was flown for 30 minutes while being irradiated with ultrasonic waves.

Next, 0.57 g of the tetrakis(triphenylphosphine) palladium was added thereto, and the mixture was heated and stirred at 68° C. for 8.5 hours. After left to cool down to room temperature, 400 ml of water was added thereto, and the organic layer was picked up by the separating operation. The organic layer was dried on the anhydrous magnesium sulfate and was, thereafter, concentrated under reduced pressure to obtain a brown crude product.

The crude product was refined by the column chromatography (carrier: silica gel, eluent: hexane/toluene), crystallized by using the mixed solvent of toluene and hexane, and was, further, reflux-washed with methanol to obtain 12.8 g of a white powder of (4′-tert-butylbiphenyl-4-yl)-(9,9-dimethyl-9H-fluorene-2-yl)-{4-(triphenylene-2-yl)phenyl}amine (compound 83) (yield, 71%).

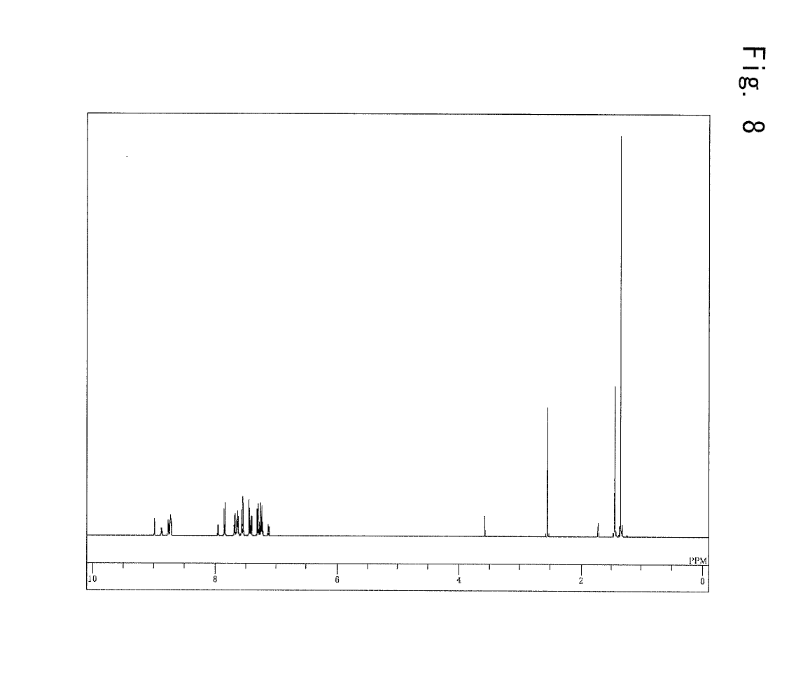

The obtained white powder was identified for its structure by the NMR. FIG. 8 shows the results of the ¹H-NMR measurement.

The following 45 signals of hydrogen were detected by the ¹H-NMR (THF-d₈).

δ (ppm)=
9.00 (1H)
8.88 (1H)
8.78-8.72 (4H)
7.96 (1H)
7.84 (2H)
7.69-7.68 (2H)
7.68-7.63 (4H)
7.58-7.55 (4H)
7.45-7.40 (4H)
7.32-7.23 (6H)
7.13 (1H)
1.45 (6H)
1.35 (9H)

### Example 9

### Synthesis of a (biphenyl-4-yl)-(4′-tert-butylbiphenyl-4-yl)-{4-(triphenylene-2-yl)phenyl}amine

### Synthesis of a Compound 84

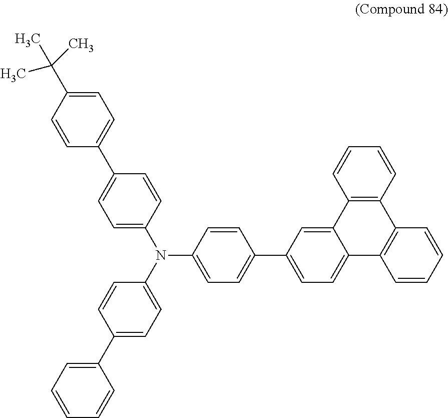 [SMILES: CC(C)(C)c1ccc(-c2ccc(N(c3ccc(-c4ccccc4)cc3)c3ccc(-c4ccc5c6ccccc6c6ccccc6c5c4)cc3)cc2)cc1]

| | | |
|---|---|---|
| (4-Bromophenyl)-(biphenyl-4-yl)-(4′-tert- | 18.1 | g, |
| butylbiphenyl-4-yl)amine | | |
| 4,4,5,5-Tetramethyl-2-(triphenylene-2-yl)- | 10.4 | g, |
| [1,3,2]dioxaborane | | |
| Toluene | 360 | ml, |
| Ethanol | 90 | ml, and |
| 2M Potassium carbonate aqueous solution | 22 | ml, |

were put into the reaction vessel in the nitrogen atmosphere, and to which the nitrogen gas was flown for 30 minutes while being irradiated with ultrasonic waves.

Next, 0.68 g of the tetrakis(triphenylphosphine) palladium was added thereto, and the mixture was heated and stirred at 74° C. for 6.5 hours. After left to cool down to room temperature, 360 ml of methanol was added thereto, and the precipitated crude product was picked up by filtration.

The crude product was dissolved in 400 ml of toluene, refined by adsorption by using 30 g of silica gel, concentrated under reduced pressure and, therefore, was crystallized by using a mixed solvent of toluene and methanol, and was reflux-washed with methanol to obtain 17.4 g of a yellow powder of (biphenyl-4-yl)-(4′-tert-butylbiphenyl-4-yl)-{4-(triphenylene-2-yl)phenyl}amine (compound 84) (yield, 83%).

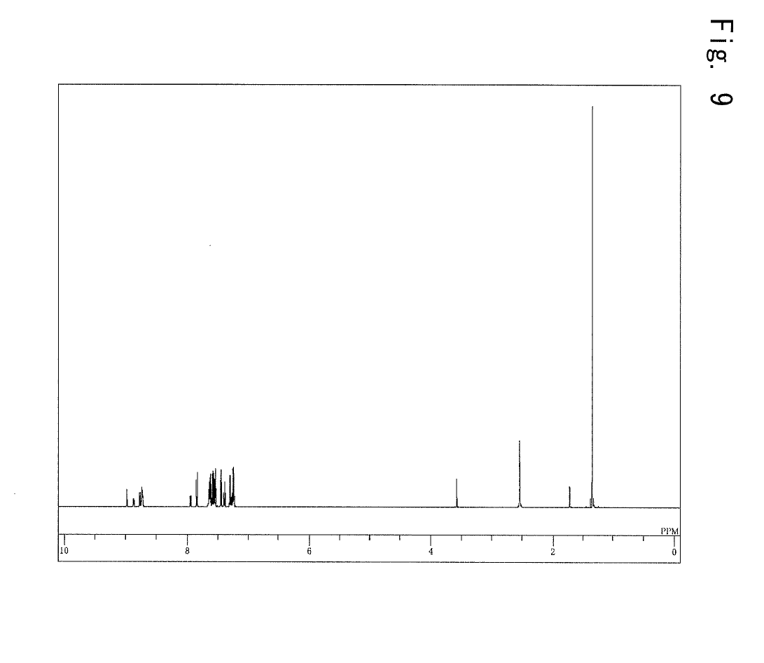

The obtained yellow powder was identified for its structure by the NMR. FIG. 9 shows the results of the ¹H-NMR measurement.

The following 41 signals of hydrogen were detected by the ¹H-NMR (THF-d₈).

δ (ppm)=
8.99 (1H)
8.88 (1H)
8.78 (1H)
7.76-7.72 (3H)
7.95 (1H)
7.84 (2H)
7.66-7.60 (6H)
7.59-7.54 (6H)
7.45 (2H)
7.40 (2H)
7.30 (2H)
7.29-7.24 (5H)
1.35 (9H)

### Example 10

### Synthesis of a (4′-tert-butylbiphenyl-4-yl)phenyl-{4-(triphenylene-2-yl)phenyl}amine

### Synthesis of a Compound 85

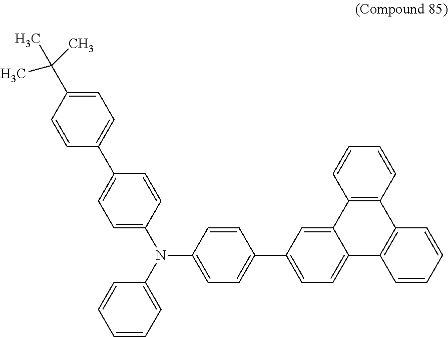 [SMILES: CC(C)(C)c1ccc(-c2ccc(N(c3ccccc3)c3ccc(-c4ccc5c6ccccc6c6ccccc6c5c4)cc3)cc2)cc1]

| | | |
|---|---|---|
| (4-Bromophenyl)-(4′-tert-butylbiphenyl-4-yl)- | 15.5 | g, |
| phenylamine | | |
| 4,4,5,5-Tetramethyl-2-(triphenylene-2-yl)- | 11.4 | g, |
| [1,3,2]dioxaborane | | |
| Toluene | 300 | ml, |
| Ethanol | 75 | ml, and |
| 2M Potassium carbonate aqueous solution | 25 | ml, |

were put into the reaction vessel in the nitrogen atmosphere, and to which the nitrogen gas was flown for 30 minutes while being irradiated with ultrasonic waves.

Next, 0.79 g of the tetrakis(triphenylphosphine) palladium was added thereto, and the mixture was heated and stirred at 68° C. for 6 hours. After left to cool down to room temperature, 300 ml of water was added thereto, and the organic layer was picked up by the separating operation. The organic layer was dried on the anhydrous magnesium sulfate and was, thereafter, concentrated under reduced pressure to obtain a black crude product.

The crude product was refined by the column chromatography (carrier: silica gel, eluent: hexane/toluene), crystallized by using the mixed solvent of toluene and methanol, and was, further, reflux-washed with methanol to obtain 12.8 g of a white powder of (4′-tert-butylphenyl-4-yl)-phenyl-{4-(triphenylene-2-yl)phenyl}amine (compound 85) (yield, 66%).

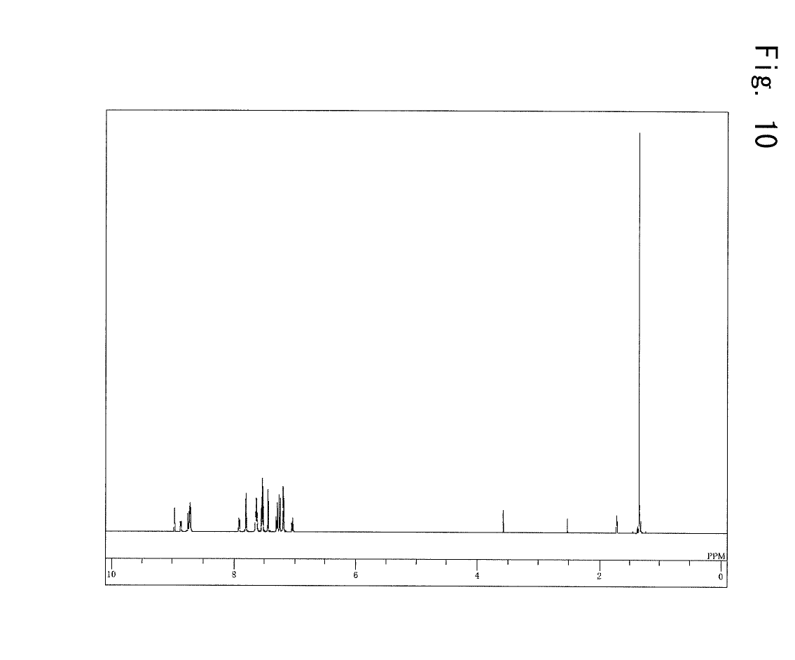

The obtained white powder was identified for its structure by the NMR. FIG. 10 shows the results of the ¹H-NMR measurement.

The following 37 signals of hydrogen were detected by the ¹H-NMR (THF-d₈).

δ (ppm)=
8.97 (1H)
8.87 (1H)
8.77-8.71 (4H)
7.92 (1H)
7.81 (2H)
7.65-7.62 (4H)
7.55-7.52 (4H)
7.44 (2H)
7.29 (2H)
7.25 (2H)
7.20-7.19 (4H)
7.05 (1H)
1.33 (9H)

### Example 11

### Synthesis of a (4′-tert-butylbiphenyl-4-yl)-{naphthalene-1-yl}-{4-(triphenylene-2-yl)phenyl}amine

### Synthesis of a Compound 86

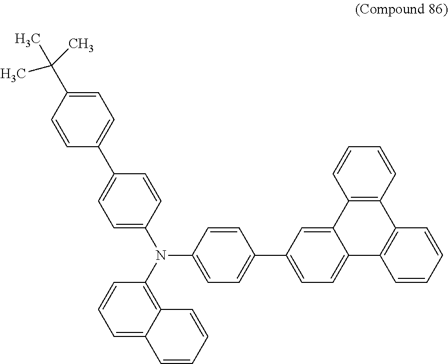 [SMILES: CC(C)(C)c1ccc(-c2ccc(N(c3ccc(-c4ccc5c6ccccc6c6ccccc6c5c4)cc3)c3cccc4ccccc34)cc2)cc1]

| | | |
|---|---|---|
| (4-Bromophenyl)-(4′-tert-butylbiphenyl-4-yl)- | 17.8 | g, |
| (naphthalene-1-yl)amine | | |
| 4,4,5,5-Tetramethyl-2-(triphenylene-2-yl)- | 10.8 | g, |
| [1,3,2]dioxaborane | | |
| Toluene | 267 | ml, |
| Ethanol | 67 | ml, and |
| 2M Potassium carbonate aqueous solution | 23 | ml, |

were put into the reaction vessel in the nitrogen atmosphere, and to which the nitrogen gas was flown for 30 minutes while being irradiated with ultrasonic waves.

Next, 0.71 g of the tetrakis(triphenylphosphine) palladium was added thereto, and the mixture was heated and stirred at 68° C. for 3 hours. After left to cool down to room temperature, 50 ml of water was added thereto, and the organic layer was picked up by the separating operation. The organic layer was dried on the anhydrous magnesium sulfate and was, thereafter, concentrated under reduced pressure to obtain a black crude product.

The crude product was refined by the column chromatography (carrier: silica gel, eluent: hexane/toluene), and was reflux-washed with methanol to obtain 11.9 g of a white powder of (4′-tert-butylbiphenyl-4-yl)-(naphthalene-1-yl)-{4-(triphenylene-2-yl)phenyl}amine (compound 86) (yield, 60%).

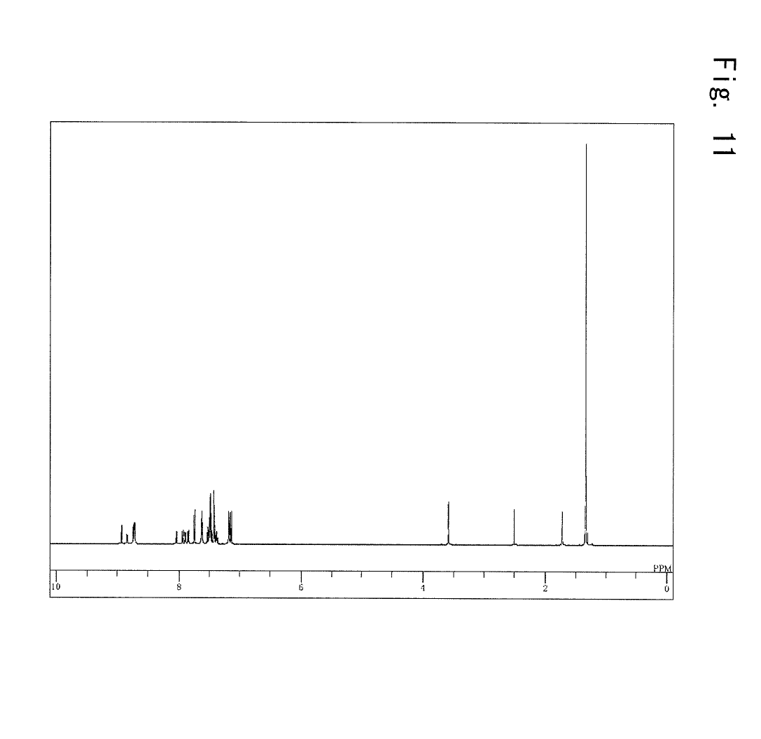

The obtained white powder was identified for its structure by the NMR. FIG. 11 shows the results of the ¹H-NMR measurement.

The following 39 signals of hydrogen were detected by the ¹H-NMR (THF-d₈).

δ (ppm)=
8.94 (1H)
8.85 (1H)
8.76-8.71 (4H)
8.04 (1H)
7.95 (1H)
7.90 (1H)
7.85 (1H)
7.75 (2H)
7.64-7.62 (4H)
7.54 (1H)
7.51-7.46 (5H)
7.43-7.40 (3H)
7.39 (1H)
7.19 (2H)
7.15 (2H)
1.34 (9H)

### Example 12

### Synthesis of a (biphenyl-4-yl)-(2-methylphenyl)-{4-(triphenylene-2-yl)phenyl}amine

### Synthesis of a Compound 87

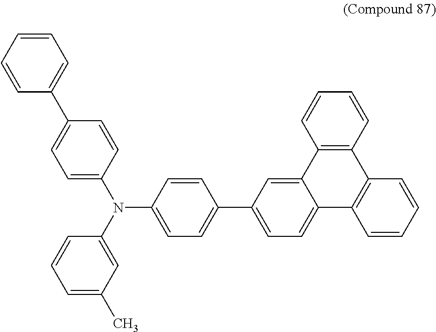 [SMILES: Cc1cccc(N(c2ccc(-c3ccccc3)cc2)c2ccc(-c3ccc4c5ccccc5c5ccccc5c4c3)cc2)c1]

| | | |
|---|---|---|
| (4-Bromophenyl)-(biphenyl-4-yl)- | 17.0 | g, |
| (2-methylphenyl)amine | | |
| 4,4,5,5-Tetramethyl-2-(triphenylene-2-yl)- | 12.5 | g, |
| [1,3,2]dioxaborane | | |
| Toluene | 255 | ml, |
| Ethanol | 64 | ml, and |
| 2M Potassium carbonate aqueous solution | 27 | ml, |

were put into the reaction vessel in the nitrogen atmosphere, and to which the nitrogen gas was flown for 30 minutes while being irradiated with ultrasonic waves.

Next, 0.82 g of the tetrakis(triphenylphosphine) palladium was added thereto, and the mixture was heated and stirred at 69° C. for 4 hours. After left to cool down to room temperature, 250 ml of water was added thereto, and the organic layer was picked up by the separating operation. The organic layer was dried on the anhydrous magnesium sulfate and was, thereafter, concentrated under reduced pressure to obtain a black crude product.

The crude product was dissolved in 400 ml of toluene, and was refined by adsorption by using 40 g silica gel. The product was concentrated under reduced pressure, crystallized by using a mixed solvent of toluene and methanol, and was reflux-washed with methanol to obtain 11.6 g of a reddish white powder of (biphenyl-4-yl)-(2-methylphenyl)-{4-(triphenylene-2-yl)phenyl}amine (compound 87) (yield, 59%).

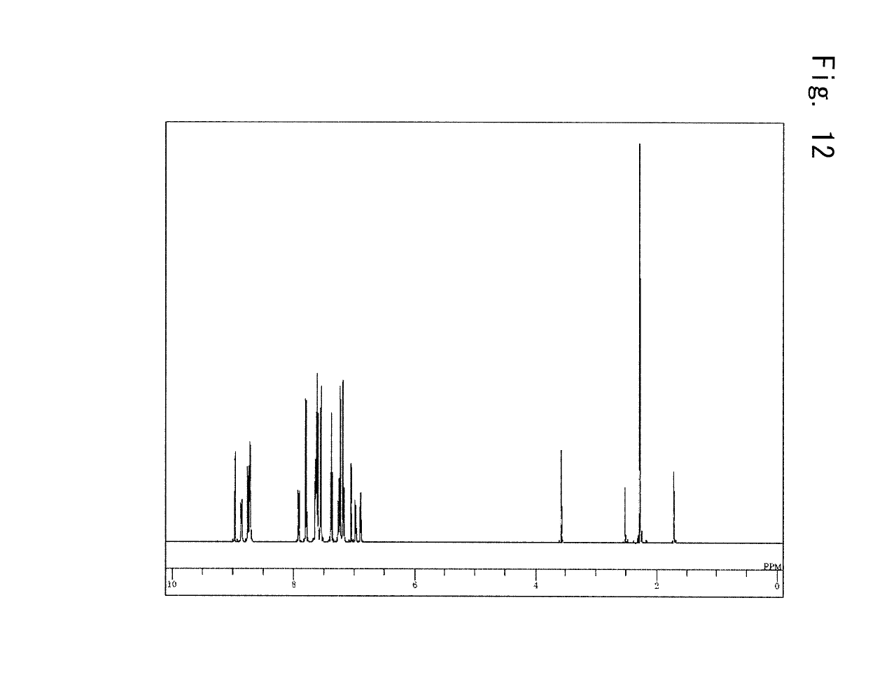

The obtained reddish white powder was identified for its structure by the NMR. FIG. 12 shows the results of the ¹H-NMR measurement.

The following 31 signals of hydrogen were detected by the ¹H-NMR (THF-d₈).

δ (ppm)=
8.97 (1H)
8.87 (1H)
8.76 (1H)
8.74-8.72 (3H)
7.93 (1H)
7.81 (2H)
7.65-7.62 (6H)
7.56 (2H)
7.39 (2H)
7.38-7.23 (3H)
7.20-7.18 (3H)
7.06 (1H)
6.98 (1H)
6.90 (1H)
2.28 (63)

### Example 13

### Synthesis of a (9,9-dimethyl-9H-fluorene-2-yl)-(naphthalene-1-yl)-{4-(triphenylene-2-il)phenyl}amine

### Synthesis of a Compound 46

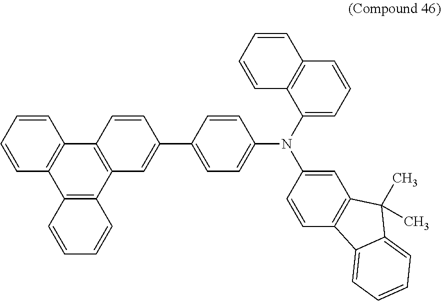 [SMILES: CC1(C)c2ccccc2-c2ccc(N(c3ccc(-c4ccc5c6ccccc6c6ccccc6c5c4)cc3)c3cccc4ccccc34)cc21]

| | | |
|---|---|---|
| (4-Bromophenyl)-(9,9-dimethyl-9H-fluorene- | 18.5 | g, |
| 2-yl)-(naphthalene-1-yl)amine | | |
| 4,4,5,5-Tetramethyl-2-(triphenylene-2-yl)- | 11.1 | g, |
| [1,3,2]dioxaborane | | |
| Toluene | 275 | ml, |
| Ethanol | 69 | ml, and |
| 2M Potassium carbonate aqueous solution | 24 | ml, |

were put into the reaction vessel in the nitrogen atmosphere, and to which the nitrogen gas was flown for 30 minutes while being irradiated with ultrasonic waves.

Next, 0.72 g of the tetrakis(triphenylphosphine) palladium was added thereto, and the mixture was heated and stirred at 69° C. for 5 hours. After left to cool down to room temperature, 270 ml of water was added thereto, and the organic layer was picked up by the separating operation. The organic layer was dried on the anhydrous magnesium sulfate and was, thereafter, concentrated under reduced pressure to obtain a black crude product.

The crude product was refined by the column chromatography (carrier: silica gel, eluent: hexane/toluene), crystallized by using the mixed solvent of toluene and hexane, and was, further, reflux-washed with methanol to obtain 7.05 g of a yellowish white powder of (9,9-dimethyl-9H-fluorene-2-yl)-(naphthalene-1-yl)-{4-(triphenylene-2-yl)phenyl}amine (compound 46) (yield, 35%).

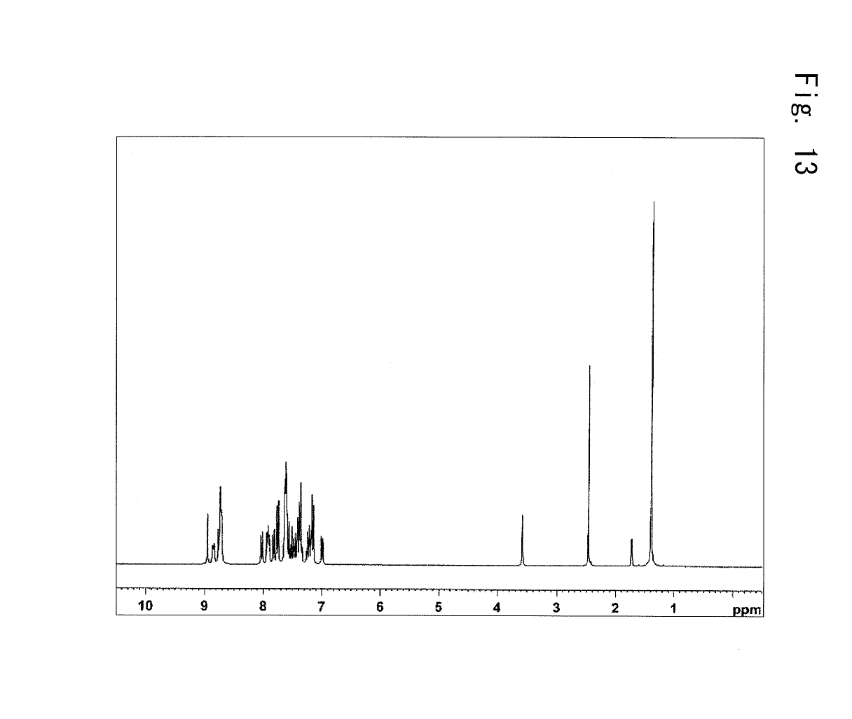

The obtained yellowish white powder was identified for its structure by the NMR. FIG. 13 shows the results of the ¹H-NMR measurement.

The following 35 signals of hydrogen were detected by the ¹H-NMR (THF-d₈).

δ (ppm)=
8.95 (1H)
8.73 (1H)
8.71-8.65 (4H)
8.02 (1H)
7.94 (2H)
7.90 (1H)
7.87 (2H)
7.82-7.36 (12H)
7.28-7.15 (4H)
7.01 (1H)
1.40 (6H)

### Example 14

### Synthesis of a (biphenyl-4-yl)-(dibenzothiophene-2-yl)-{4-(triphenylene-2-yl)phenyl}amine

### Synthesis of a Compound 88

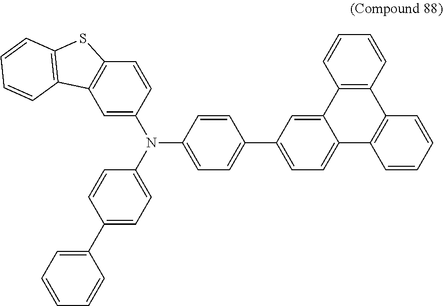 [SMILES: c1ccc(-c2ccc(N(c3ccc(-c4ccc5c6ccccc6c6ccccc6c5c4)cc3)c3ccc4sc5ccccc5c4c3)cc2)cc1]

| | | |
|---|---|---|
| (Biphenyl-4-yl)-(4-bromophenyl)- | 14.3 | g, |
| (dibenzothiophene-2-il)amine | | |
| 4,4,5,5-Tetramethyl-2-(triphenylene-2-yl)- | 10.0 | g, |
| [1,3,2]dioxaborane | | |
| Toluene | 80 | ml, |
| Ethanol | 20 | ml, and |
| 2M Potassium carbonate aqueous solution | 7.8 | ml, |

were put into the reaction vessel in the nitrogen atmosphere, and to which the nitrogen gas was flown for 60 minutes while being irradiated with ultrasonic waves.

Next, 0.65 g of the tetrakis(triphenylphosphine) palladium was added thereto, and the mixture was heated, refluxed and stirred for 10 hours. After left to cool down to room temperature, water and toluene were added thereto, and a precipitated crude product was picked up by filtration. The crude product was recrystalyzed by using 1,2-dichlorobenzene and was recrystallized again by using a mixed solvent of toluene and 1,2-dichlorobenzene to obtain 12.1 g of a faintly yellow powder of (biphenyl-4-yl)-(dibenzothiophene-2-yl)-{4-(triphenylene-2-yl)phenyl}amine (compound 88) (yield, 65%).

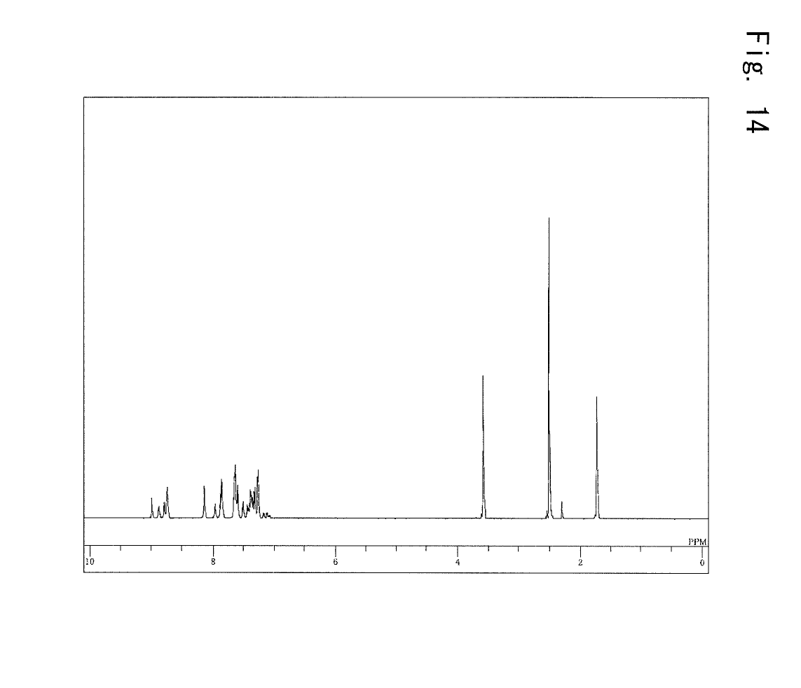

The obtained faintly yellow powder was identified for its structure by the NMR. FIG. 14 shows the results of the ¹H-NMR measurement.

The following 31 signals of hydrogen were detected by the ¹H-NMR (THF-d₈).

δ (ppm)=
8.99 (1H)
8.87 (1H)
8.79-8.74 (4H)
8.14 (2H)
7.96 (1H)
7.88-7.85 (4H)
7.65-7.58 (7H)
7.50 (1H)
7.43-7.26 (10H)

### Example 15

### Synthesis of a (dibenzothiophene-2-yl)-(9,9-dimethyl-9H-fluorene-2-yl)-{4-(triphenylene-2-yl)phenyl}amine

### Synthesis of a Compound 89

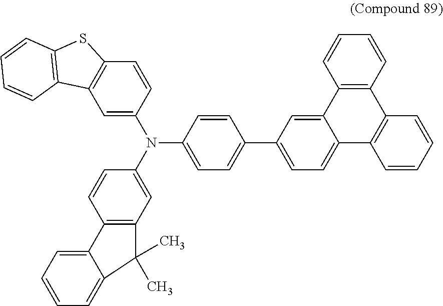 [SMILES: CC1(C)c2ccccc2-c2ccc(N(c3ccc(-c4ccc5c6ccccc6c6ccccc6c5c4)cc3)c3ccc4sc5ccccc5c4c3)cc21]

| | | |
|---|---|---|
| (4-Bromophenyl)-(dibenzothiophene-2-yl)- | 15.6 | g, |
| (9,9-dimethyl-9H-fluorene-2-yl)amine | | |
| 4,4,5,5-Tetramethyl-2-(triphenylene-2-yl)- | 10.2 | g, |
| [1,3,2]dioxaborane | | |
| Toluene | 80 | ml, |
| Ethanol | 20 | ml, and |
| 2M Potassium carbonate aqueous solution | 8 | ml, |

were put into the reaction vessel in the nitrogen atmosphere, and to which the nitrogen gas was flown for 60 minutes while being irradiated with ultrasonic waves.

Next, 0.67 g of the tetrakis(triphenylphosphine) palladium was added thereto, and the mixture was heated, refluxed and stirred for 4 hours. After left to cool down to room temperature, water and toluene were added thereto, and a precipitated crude product was picked up by filtration. The crude product was recrystallized by using a mixed solvent of toluene, 1,2-dichlorobenzene and ethyl acetate, and was reflux-washed with methanol to obtain 10.6 g of a faintly yellow powder of (dibenzothiophene-2-yl)-(9,9-dimethyl-9H-fluorene-2-yl)-{4-(triphenylene-2-yl)phenyl}amine (compound 89) (yield, 53%).

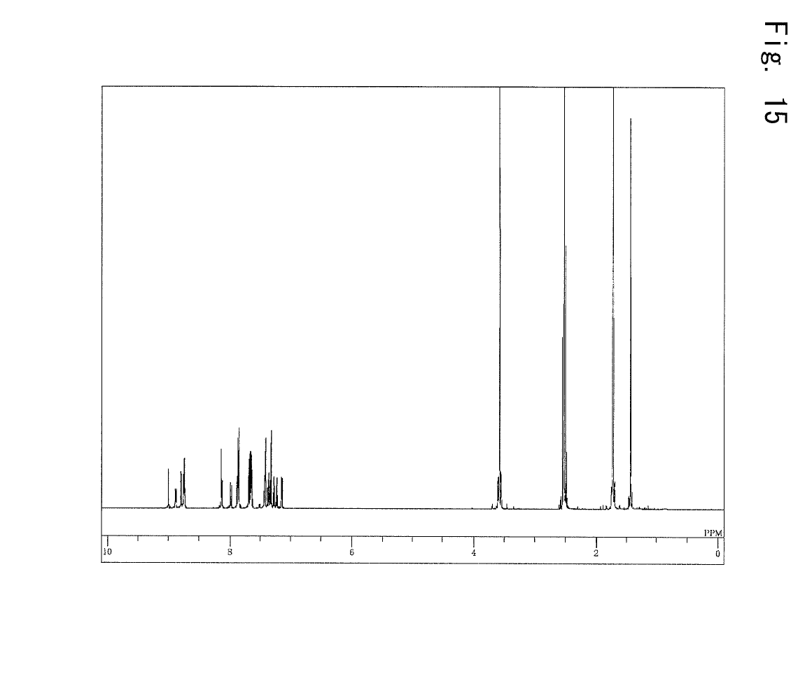

The obtained faintly yellow powder was identified for its structure by the NMR. FIG. 15 shows the results of the ¹H-NMR measurement.

The following 35 signals of hydrogen were detected by the ¹H-NMR (THF-d₈).

δ (ppm)=
9.00 (1H)
8.89 (1H)
8.80 (1H)
7.78-7.74 (3H)
8.13-8.11 (2H)
7.99 (1H)
7.88-7.84 (4H)
7.69-7.63 (6H)
7.44-7.42 (3H)
7.41-7.38 (4H)
7.27 (1H)
7.23 (1H)
7.14 (1H)
1.42 (6H)

### Example 16

### Synthesis of a bis(9,9-dimethyl-9H-fluorene-2-yl)-{4-(triphenylene-2-yl)phenyl}amine

### Synthesis of a Compound 90

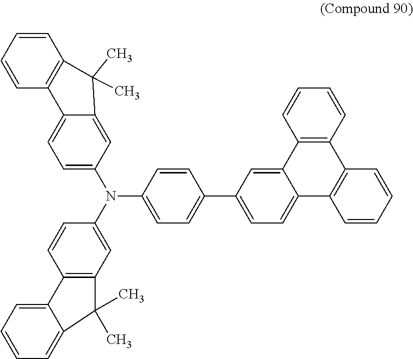 [SMILES: CC1(C)c2ccccc2-c2ccc(N(c3ccc(-c4ccc5c6ccccc6c6ccccc6c5c4)cc3)c3ccc4c(c3)C(C)(C)c3ccccc3-4)cc21]

| | | |
|---|---|---|
| Bis(9,9-dimethyl-9H-fluorene-2-yl)- | 15.8 | g, |
| (4-bromophenyl)amine | | |
| 4,4,5,5-Tetramethyl-2-(triphenylene-2-yl)- | 9.6 | g, |
| [1,3,2]dioxaborane | | |
| Toluene | 800 | ml, |
| Ethanol | 20 | ml, and |
| 2M Potassium carbonate aqueous solution | 7.9 | ml, |

were put into the reaction vessel in the nitrogen atmosphere, and to which the nitrogen gas was flown for 30 minutes while being irradiated with ultrasonic waves.

Next, 0.66 g of the tetrakis(triphenylphosphine) palladium was added thereto, and the mixture was heated, refluxed and stirred for 4 hours. After left to cool down to room temperature, 300 ml of water was added thereto, and the organic layer was picked up by the separating operation. The organic layer was dried on the anhydrous magnesium sulfate and was concentrated under reduced pressure to obtain a brown crude product.

The crude product was dissolved in toluene, refined by adsorption by using 60 g of silica gel, refined by adsorption again by using 10 g of active carbon, crystallized by using a mixed solvent of toluene, acetone and methanol, and was crystallized again by using a mixed solvent of toluene and hexane. Thereafter, the product was washed with toluene heated at 70° C., dissolved in 1,2-dichloromethane, refined by adsorption by using NH silica gel and was, further, crystallized by using hexane to obtain 10.2 g of a faintly yellow powder of bis(9,9-dimethyl-9H-fluorene-2-yl)-{4-(triphenylene-2-yl)phenyl}amine (compound 90) (yield, 51%).

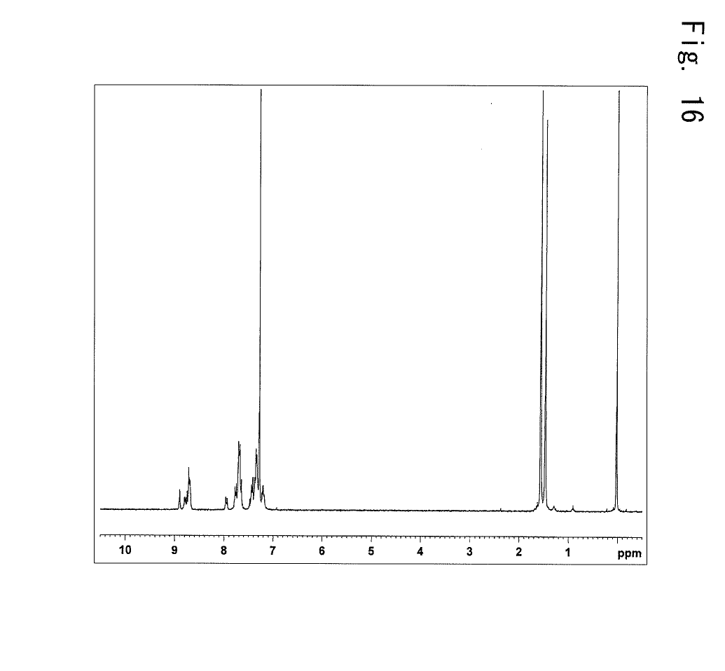

The obtained faintly yellow powder was identified for its structure by the NMR. FIG. 16 shows the results of the ¹H-NMR measurement.

The following 41 signals of hydrogen were detected by the ¹H-NMR (THF-d₈).

δ (ppm)=
8.92 (1H)
8.82-8.65 (5H)
7.95 (1H)
7.81-7.62 (10H)
7.49-7.16 (12H)
1.43 (12H)

### Example 17

By using a highly sensitive differential scanning calorimeter (DSC 3100S manufactured by Bruker AXS Co.), the compounds (triphenylene derivatives) obtained in the above Examples 1 to 16 were measured for their melting points and glass transition points. The results were as follows:

| | | Glass transition |
|---|---|---|
| | Melting points | points |
| Compound of | 257° C. | 116° C. |
| Example 1 | | |
| Compound of | 236° C. | 115° C. |
| Example 2 | | |
| Compound of | 160° C. | 131° C. |
| Example 3 | | |
| Compound of | not measured | 129° C. |
| Example 4 | | |
| Compound of | 236° C. | 116° C. |
| Example 5 | | |
| Compound of | not measured | 115° C. |
| Example 6 | | |
| Compound of | not measured | 134° C. |
| Example 7 | | |
| Compound of | 245° C. | 145° C. |
| Example 8 | | |
| Compound of | 160° C. | 133° C. |
| Example 9 | | |
| Compound of | 145° C. | 117° C. |
| Example 10 | | |
| Compound of | 172° C. | 139° C. |
| Example 11 | | |
| Compound of | 129° C. | 98° C. |
| Example 12 | | |
| Compound of | not measured | 136° C. |
| Example 13 | | |
| Compound of | 166° C. | 133° C. |
| Example 14 | | |
| Compound of | 320° C. | 149° C. |
| Example 15 | | |
| Compound of | 272° C. | 147° C. |
| Example 16 | | |

The compounds obtained in Examples 1 to 16 have glass transition points which are not lower than 95° C. indicating that the thin films formed by using the compounds of the invention maintain stability.

### Example 18

By using the compounds of the invention obtained in Examples 1 to 5, 8 to 12 and 14 to 16, films were vapor-deposited in a thickness of 100 nm on an ITO substrate and were measured for their work functions in the atmosphere by using a photoelectron spectroscope (Model AC-3 manufactured by Riken Keiki Co.). The results were as follows:

| | Work functions |
|---|---|
| Compound of | 5.62 eV |
| Example 1 | |
| Compound of | 5.57 eV |
| Example 2 | |
| Compound of | 5.61 eV |
| Example 3 | |
| Compound of | 5.37 eV |
| Example 4 | |
| Compound of | 5.56 eV |
| Example 5 | |
| Compound of | 5.46 eV |
| Example 8 | |
| Compound of | 5.56 eV |
| Example 9 | |
| Compound of | 5.63 eV |
| Example 10 | |
| Compound of | 5.63 eV |
| Example 11 | |
| Compound of | 5.60 eV |
| Example 12 | |
| Compound of | 5.61 eV |
| Example 14 | |
| Compound of | 5.56 eV |
| Example 15 | |
| Compound of | 5.46 eV |
| Example 16 | |

By using the compounds of the invention obtained in Examples 6, 7 and 13, films were vapor-deposited in a thickness of 100 nm on the ITO substrate and were measured for their work functions by using an ionization potential-measuring device (PYS-202 manufactured by Sumitomo Heavy Machinery Industries Co.). The results were as follows:

| | Work functions |
|---|---|
| Compound of | 5.57 eV |
| Example 6 | |
| Compound of | 5.62 eV |
| Example 7 | |
| Compound of | 5.63 eV |
| Example 13 | |

From the above results, it will be learned that the triphenylene derivatives of the present invention have favorable energy levels as compared to the work function of 5.4 eV possessed by general hole-transporting materials such as NPD, TPD and the like, and have good hole-blocking powers.

### Example 19

An organic EL device of a layer structure shown in FIG. 17 was fabricated by vapor-depositing a hole injection layer 3, a hole-transporting layer 4, a luminous layer 5, an electron-transporting layer 6, an electron injection layer 7 and a cathode (aluminum electrode) 8 in this order on a glass substrate 1 on which an ITO electrode has been formed in advance as a transparent anode 2.

Concretely, the glass substrate 1 on which the ITO film has been formed in a thickness of 150 nm was washed with an organic solvent and was, thereafter, washed on its surfaces by an oxygen plasma treatment. Thereafter, the glass substrate with the ITO electrode was placed in a vacuum evaporation machine, and the pressure therein was reduced down to 0.001 Pa or lower.

Next, as the hole injection layer 3, a compound 115 of the following structural formula was formed in a thickness of 20 nm so as to cover the transparent anode 2.

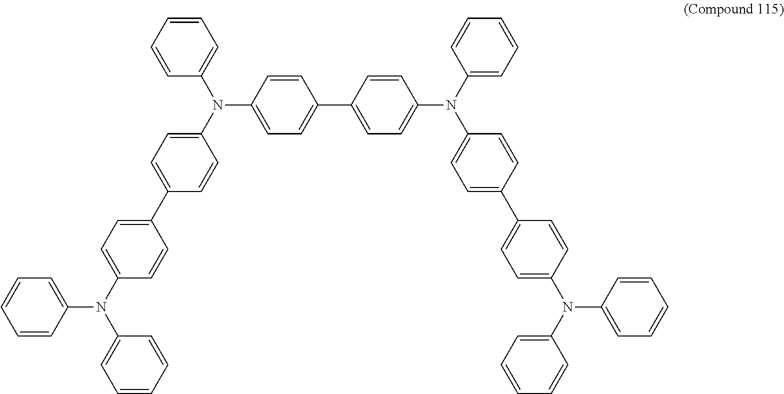 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

On the hole injection layer 3, the compound of Example 2 (compound 15) was deposited in a thickness of 40 nm to form the hole-transporting layer 4.

On the hole-transporting layer 4, the luminous layer 5 was formed in a thickness of 30 nm by two-way-depositing a compound 116 of the following structural formula and a compound 117 of the following structural formula at such deposition rates that the ratio of the deposition rates was compound 116:compound 117=5:95. On the luminous layer 5 was further formed the electron-transporting layer 6 by depositing Alq₃ in a thickness of 30 nm.

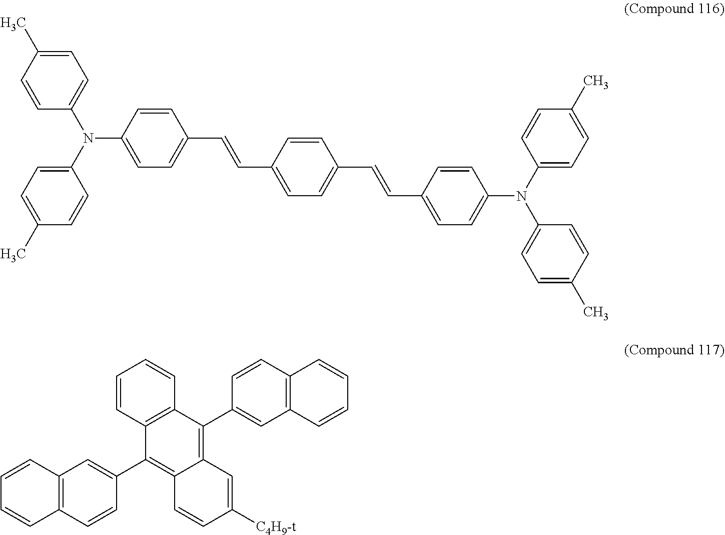

On the electron-transporting layer 6 was further formed the electron injection layer 7 by depositing lithium fluoride in a thickness of 0.5 nm.

Finally, aluminum was vapor-deposited thereon in a thickness of 150 nm to form the cathode 8.

The organic EL device fabricated by using the compound of Example 2 (compound 15) of the invention was measured for its properties in the atmosphere at normal temperature.

Concretely, the organic EL device was impressed with a DC voltage to measure the luminous properties. The results were as shown in Table 1.

### Example 20

An organic EL device was fabricated in the same manner as in Example 19 but using the compound of Example 1 (compound 66) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 21

An organic EL device was fabricated in the same manner as in Example 19 but using the compound of Example 3 (compound 67) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 22

An organic EL device was fabricated in the same manner as in Example 19 but using the compound of Example 4 (compound 79) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 23

An organic EL device was fabricated in the same manner as in Example 19 but using the compound of Example 5 (compound 80) as the material of the hole-transporting layer 4 and forming the hole-transporting layer 4 in a thickness of 40 nm.

The organic EL device was impressed with a DC voltage to measure the luminous properties in the same manner as in Example 19. The results were as shown in Table 1.

### Example 24

An organic EL device was fabricated in the same manner as in Example 19 but using the compound of Example 6 (compound 81) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 25

An organic EL device was fabricated in the same manner as in Example 19 but using the compound of Example 7 (compound 82) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 26

An organic EL device was fabricated in the same manner as in Example 19 but using the compound of Example 8 (compound 83) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 27

An organic EL device was fabricated in the same manner as in Example 19 but using the compound of Example 9 (compound 84) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 28

An organic EL device was fabricated in the same manner as in Example 19 but using the compound of Example 10 (compound 85) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 29

An organic EL device was fabricated in the same manner as in Example 19 but using the compound of Example 11 (compound 86) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 30

An organic EL device was fabricated in the same manner as in Example 19 but using the compound of Example 12 (compound 87) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 31

An organic EL device was fabricated in the same manner as in Example 19 but using the compound of Example 13 (compound 46) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 32

An organic EL device was fabricated in the same manner as in Example 19 but using the compound of Example 14 (compound 88) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 33

An organic EL device was fabricated in the same manner as in Example 19 but using the compound of Example 15 (compound 89) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Example 34

An organic EL device was fabricated in the same manner as in Example 19 but using the compound of Example 16 (compound 90) as the material of the hole-transporting layer 4 and forming the hole-transporting layer 4 in a thickness of 40 nm.

The organic EL device was impressed with the DC voltage to measure the luminous properties in the same manner as in Example 19. The results were as shown in Table 1.

### Comparative Example 1

For comparison, an organic EL device was fabricated in the same manner as in Example 19 but using, as the material of the hole-transporting layer 4, a compound 118 of the following structural formula instead of using the compound of Example 2, and forming the hole-transporting layer 4 in a thickness of 40 nm.

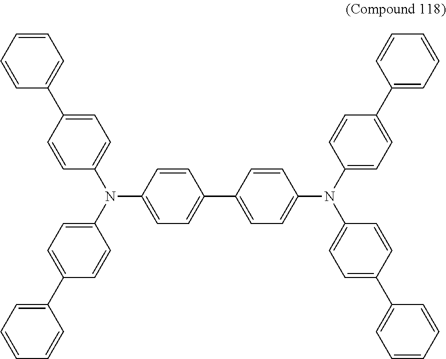 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

The organic EL device was impressed with the DC voltage to measure the luminous properties in the same manner as in Example 19. The results were as shown in Table 1.

TABLE 1

| | | | | Luminous | Power |
|---|---|---|---|---|---|
| | | Voltage {V} | Brightness [cd/m²] | efficiency [cd/A] | efficiency [lm/W] |
| | Compound | (@10 mA/cm²) | (@10 mA/cm²) | (@10 mA/cm²) | (@ 10 mA/cm²) |
| Ex. 19 | compound 15 | 4.73 | 1028 | 10.3 | 6.84 |
| Ex. 20 | compound 66 | 4.97 | 963 | 9.63 | 6.09 |
| Ex. 21 | compound 67 | 5.08 | 963 | 9.64 | 5.97 |
| Ex. 22 | compound 79 | 4.82 | 959 | 9.60 | 6.26 |
| Ex. 23 | compound 80 | 4.96 | 909 | 9.10 | 5.76 |
| Ex. 24 | compound 81 | 4.77 | 962 | 9.63 | 6.34 |
| Ex. 25 | compound 82 | 4.78 | 959 | 9.60 | 6.33 |
| Ex. 26 | compound 83 | 4.88 | 923 | 9.23 | 5.95 |
| Ex. 27 | compound 84 | 5.15 | 916 | 9.16 | 5.59 |
| Ex. 28 | compound 85 | 5.11 | 903 | 9.03 | 5.55 |
| Ex. 29 | compound 86 | 5.01 | 957 | 9.57 | 6.00 |
| Ex. 30 | compound 87 | 5.15 | 923 | 9.24 | 5.64 |
| Ex. 31 | compound 46 | 4.99 | 962 | 9.63 | 6.07 |
| Ex. 32 | compound 88 | 5.10 | 938 | 9.38 | 5.78 |
| Ex. 33 | compound 89 | 4.95 | 928 | 9.28 | 5.89 |
| Ex. 34 | compound 90 | 4.83 | 949 | 9.50 | 6.18 |
| Comp. | compound 118 | 5.17 | 902 | 9.03 | 5.49 |
| Ex. 1 | | | | | |

As for the driving voltage at a current density of 10 mA/cm² as shown in Table 1, the compounds of Examples 1 to 16 of the invention have driving voltages of as low as 4.73 to 5.15 V as compared to 5.17 V of the compound 118 (Comparative Example 1).

Moreover, Examples 1 to 16 all show power efficiencies of from 5.55 to 6.84 lm/W which are great improvements over 5.49 lm/W of Comparative Example 1.

It will, therefore, be learned that the organic EL device having an organic layer formed by using the triphenylene derivative of the present invention features improved luminous efficiency and power efficiency as compared to the organic EL device that uses the known compound 118, and is capable of achieving a decrease in the practical driving voltage.

### INDUSTRIAL APPLICABILITY

The triphenylene derivatives of the present invention have a high hole-transporting capability and excellent amorphous property maintaining stability in their thin-film forms, and can be used as excellent compounds for fabricating the organic EL devices. Upon fabricating the organic EL devices by using the above compounds, further, it is allowed to attain a high luminous efficiency and power efficiency while lowering the practical driving voltage and improving the durability. Their use can, therefore, be expanded to, for example, domestic appliances and illumination equipment.

### DESCRIPTION OF SYMBOLS

1 glass substrate
2 transparent anode
3 hole injection layer
4 hole-transporting layer
5 luminous layer
6 electron-transporting layer
7 electron injection layer
8 cathode

1. Triphenylene derivatives represented by the following general formula (1), 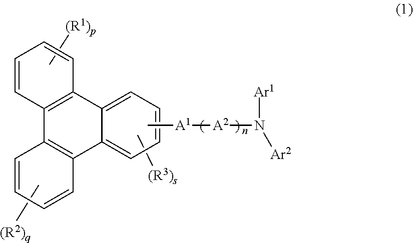
wherein,
p and q are, respectively, integers of 0 or 1 to 4,
s is an integer of 0 or 1 to 3,
n is an integer of 0, 1 or 2,
Ar¹ and Ar² are, respectively, aromatic hydrocarbon groups or aromatic heterocyclic groups and, wherein, Ar¹ and Ar² may form a ring by being bonded together via a single bond, via a methylene group that may have a substituent, via an oxygen atom or via a sulfur atom,
R¹, R² and R³ are, respectively, deuterium atoms, fluorine atoms, chlorine atoms, cyano groups, nitro groups, alkyl groups having 1 to 6 carbon atoms, cycloalkyl groups having 5 to 10 carbon atoms, alkyloxy groups having 1 to 6 carbon atoms, cycloalkyloxy groups having 5 to 10 carbon atoms, aromatic hydrocarbon groups, aromatic heterocyclic groups or aryloxy groups,
A¹ and A² are, respectively, divalent aromatic hydrocarbon groups or divalent aromatic heterocyclic groups,
if n is 0, then A¹ and Ar¹ may form a ring by being bonded together via a single bond, via a methylene group that may have a substituent, via an oxygen atom or via a sulfur atom,
if n is 1, then A¹ or A² and Ar¹ may form a ring by being bonded together via a single bond, via a methylene group that may have a substituent, via an oxygen atom or via a sulfur atom, and
if n is 2, then the plurality of A² may be different from each other, and A¹ or A² and Ar¹ may form a ring by being bonded together via a single bond, via a methylene group that may have a substituent, via an oxygen atom or via a sulfur atom.

2. The triphenylene derivatives according to claim 1, wherein n is 0 in the general formula (1).

3. The triphenylene derivatives according to claim 1, wherein the divalent group A¹ in the general formula (1) is a phenylene group that may have a substituent.

4. The triphenylene derivatives according to claim 1 represented by the following general formula (1a), 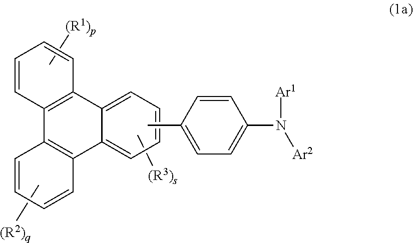
wherein,
p, q, s, Ar¹, Ar² and R¹ to R³ are as defined in the general formula (1).

5. The triphenylene derivatives according to claim 1, wherein the divalent group A¹ is bonded to the second position of the triphenylene ring.

6. The triphenylene derivatives according to claim 5 represented by the following general formula (1b), 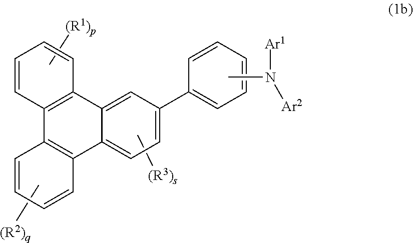
wherein,
p, q, s, Ar¹, Ar² and R¹ to R³ are as defined in the general formula (1).

7. The triphenylene derivatives according to claim 6 represented by the following general formula (1b-1), 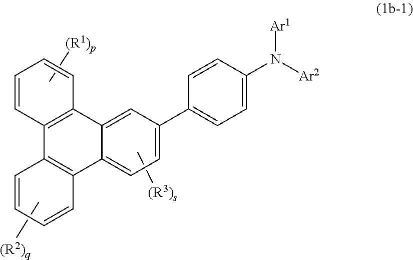
wherein,
p, q, s, Ar¹, Ar² and R¹ to R³ are as defined in the general formula (1).

8. An organic electroluminescent device having a pair of electrodes and at least one organic layer interposed therebetween, wherein
at least one of the organic layers contains the triphenylene derivative of claim 1.

9. The organic electroluminescent device according to claim 8, wherein the organic layer containing the triphenylene derivative is a hole-transporting layer.

10. The organic electroluminescent device according to claim 8, wherein the organic layer containing the triphenylene derivative is an electron-blocking layer.

11. The organic electroluminescent device according to claim 8, wherein the organic layer containing the triphenylene derivative is a hole injection layer.

12. The organic electroluminescent device according to claim 8, wherein the organic layer containing the triphenylene derivative is a luminous layer.
